(19) United States
(12) Patent Application Publication
(10) Pub. No.: US 2022/0288253 A1
(43) Pub. Date: Sep. 15, 2022

(54) VIRUCIDAL EFFECTS OF 405 NM VISIBLE LIGHT ON SARS-COV2 AND INFLUENZA A VIRUS

(71) Applicant: KENALL MANUFACTURING COMPANY, Kenosha, WI (US)

(72) Inventor: Clifford J. Yahnke, Kenosha, WI (US)

(21) Appl. No.: 17/692,984

(22) Filed: Mar. 11, 2022

Related U.S. Application Data
(60) Provisional application No. 63/160,331, filed on Mar. 12, 2021.

(51) Int. Cl. A61L 2/10; A61L 2/26; A61L 9/20

(52) U.S. Cl. CPC A61L 2/10; A61L 2/26; A61L 9/20; A61L 2209/12; A61L 2202/11

(57) ABSTRACT

A lighting device and methods thereof to inactivate viruses in an environment. Particularly, systems and methods demonstrate the virucidal effects of 405 nm irradiation to inactivate viruses including SARS-CoV-2 and influenza A H1N1 virus, specifically in the absence of exogenous photosensitizers, despite previous efforts in the field suggesting the need for one or more photosensitizers to achieve successful inactivating effect. Moreover, systems and methods implement the 405 nm in the context of lighting devices that may disinfect an environment while providing a combined light output that is unobjectionable to humans.

### CROSS REFERENCE TO RELATED APPLICATIONS

This application claims priority to, and the benefit of the filing date of, U.S. Provisional Patent Application No. 63/160,331, entitled “Virucidal effects of 405 nm visible light on SARS-CoV2 and influenza A virus” and filed on Mar. 12, 2021, the entire disclosure of which is hereby incorporated by reference herein.

### FIELD

The present disclosure generally relates to a lighting system, and more particularly, to a lighting system that uses visible light (e.g., 405 nm visible light) to inactivate viruses, such as coronaviruses and influenza viruses (e.g., SARS-CoV-2 and influenza A viruses).

### BACKGROUND

The severe acute respiratory syndrome corona virus 2 (SARS-CoV-2), the causative agent of the COVID-19 pandemic, is a member of the beta-coronavirus family. SARS-CoV-2 emerged at the end of 2019 in the Chinese city of Wuhan, the capital of China's Hubei province (Andersen, K. G., Rambaut, A., Lipkin, W. I. et al. The proximal origin of SARS-CoV-2. Nat. Med. 26, 450-452 (2020)). By late February 2021, more than 112 million cases of SARS-CoV-2 had been reported, while accounting for approximately 2.5 million deaths, underscoring the rapid dissemination of the virus on a global scale (Worldometer, D. COVID-19 coronavirus pandemic. World Health Organization (2020)). As a complement to standard precautions such as handwashing, masking, surface disinfection, and social distancing, still other enhancements to enclosed spaces have been proposed to mitigate the spread of SARS-CoV-2. These enhancements have been considered in a multiplicity of settings, including healthcare environments, retail environments, dining environments, and transportation environments (Buitrago-Garcia, D., Egli-Gany, D., Counotte, M. J., Hossmann, S., Imeri, H., Ipekci, A. M. et al. Occurrence and transmission potential of asymptomatic and presymptomatic SARS-CoV-2 infections: A living systematic review and meta-analysis. PLoS Medicine 17(9): e1003346 (2020)).

Initial guidance from health authorities such as the United States Centers for Disease Control and Prevention (CDC) and the World Health Organization (WHO) on environmental transmission of SARS-CoV-2 focused on contaminated surfaces as fomites (“Modes of Transmission of Virus Causing COVID-19: Implications for IPC Precaution Recommendations.” World Health Organization, World Health Organization, 29 Mar. 2020). Data pertaining to the survival of SARS-CoV-2 and other related coronaviruses have indicated that virions are able to persist on fomites composed of plastic, wood, paper, metal, and glass, for potentially as long as nine days (Dehbandi, R. & Zazouli, M. A. Stability of SARS-CoV-2 in different environmental conditions. The Lancet Microbe 1, e145 (2020); Van Doremalen, N. et al. Aerosol and surface stability of SARS-CoV-2 as compared with SARS-CoV-1. N Engl. J. Med. 382, 1564-1567 (2020); Behzadinasab, S., Chin, A., Hosseini, M., Poon, L. & Ducker, W. A. A surface coating that rapidly inactivates SARS-CoV-2. ACS applied materials & interfaces 12, 34723-34727 (2020); Chan, K. et al. Factors affecting stability and infectivity of SARS-CoV-2. J. Hosp. Infect. 106, 226-231 (2020)). Some later studies have suggested that SARS-CoV-2 may remain viable in such surfaces for approximately at least three days, and another two studies showed that at room temperature (20-25° Celsius (C)), a 14-day time period was required to see a 4.5-5 log₁₀ reduction of the virus (Biryukov, J. et al. Increasing Temperature and Relative Humidity Accelerates Inactivation of SARS-CoV-2 on Surfaces. mSphere 6, 10.1128/mSpheree.00441-20 (2020); Aboubakr, H. A., Sharafeldin, T. A. & Goyal, S. M. Stabliity of SARS-CoV-2 and other coronaviruses in the environment and on common touch surfaces and the influence of climatic conditions: A review. Transboundary and emerging diseases (2020)).

Since the start of the SARS-CoV-2 pandemic, we have learned that transmission of the virus may occur by way of respiratory droplets and aerosols, but the relative impact of each mode of transmission has been the subject of much debate. Nevertheless, enclosed spaces with groups of people exercising or singing have been found to be associated with increased virus transmission. The half-life survival of SARS-CoV-2 in this type of environment has been estimated to be between one and two hours (Van Doremalen et al., 2020; Smither, S. J., Eastaugh, L. S., Findlay, J. S. & Lever, M. S. Experimental aerosol survival of SARS-CoV-2 in artificial saliva and tissue culture media at medium and high humidity. Emerging microbes & infections 9, 1415-1417 (2020); Schuit, M. et al. Airborne SARS-CoV-2 is rapidly inactivated by simulated sunlight. J. Infect. Dis. 222, 564-571 (2020)).

Taking this information into consideration, several methods have been evaluated to effectively inactivate SARS-CoV-2. Chemical methods, which focus on surface disinfection, utilize 70% alcohol and bleach, and the benefits of these methods are well established. These methods are also episodic (or non-continuous), meaning that in between applications of the methods, the environment is not being treated (Kampf, G., Todt, D., Pfaender, S. & Steinmann, E. Persistence of coronaviruses on inanimate surfaces and their inactivation with biocidal agents. J. Hosp. Infect. 104, 246-251 (2020)). In addition to chemical methods, one of the most-utilized methods for whole-room disinfection is the application of germicidal ultra-violet C light (UVC; ˜254 nanometer (nm) wavelength) (Rutala, W. A. & Weber, D. J. Disinfection and sterilization in health care facilities: what clinicians need to know. Clinical infectious diseases 39, 702-709 (2004)). This technology is well-established and has been shown to inactivate a range of pathogens including bacteria, fungi, and viruses (Rathnasinghe, R. et al. Scalable, effective, and rapid decontamination of SARS-CoV-2 contaminated N95 respirators using germicidal ultra-violet C (UVC) irradiation device. medRxiv (2020); Escombe, A. R. et al. Upper-room ultraviolet light and negative air ionization to prevent tuberculosis transmission. PLoS Med 6, e1000043 (2009); Napkan, W., Yermakov, M., Indugula, R., Reponen, T. & Grinshpun, S. A. Inactivation of bacterial and fungal spores by UV irradiation and gaseous iodine treatment applied to air handling filters. Sci. Total Environ. 671, 59-65 (2019); Tseng, C. & Li, C. Inactivation of viruses on surfaces by ultraviolet germicidal irradiation. Journal of occupational and environmental hygiene 4, 400-405 (2007)). The mechanism of action of UVC is photodimerization of genetic material such as RNA (relevant for SARS-CoV-2 and influenza A virus (IAV)) and DNA (relevant for DNA viruses and bacterial pathogens, among others) (Kowalski, W. in Ultraviolet germicidal irradiation handbook: UVGI for air and surface disinfection (Springer science & business media, 2010). Unfortunately, however, this method has been associated with deleterious effects in humans exposed to UVC, such effects including photokeratoconunctivitis in eyes and photodermatitis in skin (Zaffina, S. et al. Accidental exposure to UV radiation produced by germicidal lamp: case report and risk assessment. Photochem. Photobiol. 88, 1001-1004 (2012)). For at least these reasons, UVC irradiation requires safety precautions and cannot be used to decontaminate fomites and high contact areas in the presence of humans (Leung, K. C. P. & Ko, T. C. S. Improper Use of the Germicidal Range Ultraviolet Lamp for Household Disinfection Leading to Phototoxicity in COVID-19 Suspects. Cornea 40, 121-122 (2021)).

Other decontamination methods involving the application of light have shown to be effective to inactivate certain types of bacteria. For example, visible violet light in the wavelength range of 400-420 nm has been demonstrated to effectively inactivate Methicillin-resistant Staphylococcus aureus (MRSA) among other bacteria, particularly when said 400-420 nm light is applied at a power that achieves an irradiance of at least 0.01 milliwatt per square centimeter (mW/cm²) as measured at a surface where the MRSA bacteria is to be inactivated. Lighting fixtures and methods associated therewith have been described, for example, in U.S. Pat. No. 15/178,349, and in U.S. Pat. No. 16/027,107, each of which is hereby incorporated by reference herein in its entirety.

Endeavors have been made to inactivate viruses by applying similar wavelengths of light. However, any success in these endeavors has required that the virus be suspended in one or more photosensitizers for any substantial viral reduction to be achieved. Tomb et al., for example, demonstrated the use of 405 nm light at an irradiance of 155.8 milliwatts per square centimeter (mW/cm²) to inactivate feline calicivirus (FCV) when the virus was suspended in an organically-rich media (ORM) having photosensitive components, and alternatively, suspended in a “minimal medium” (MM) lacking photosensitive components. For the FCV sample suspended in MM and subject to the irradiance of 155.8 mW/cm², one hour of exposure (a total irradiating energy of 561 Joules per square centimeter (J/cm²)) achieved a less than 1.0 log₁₀ reduction, and a full five hours (2804 J/cm²) of exposure at the same irradiance were required to achieve a 3.9 log₁₀ reduction (Tomb, R. M. et al. New proof-of-concept in viral inactivation: virucidal efficacy of 405 nm light against feline calicivirus as a model for norovirus decontamination. Food and environmental virology 9, 159-167). The magnitude of 405 nm irradiance and total irradiating energy required to achieve these effects was significant and, as such, casted doubt on the practicability of inactivating viral pathogens without the use of an external or exogenous photosensitizer, as a lighting device in a practical setting (e.g., hospital, restaurant, etc.) would not likely be able to safely apply nearly this magnitude of 405 nm light in an environment that is to be occupied by humans, as the necessary source power would exceed the 405 nm exposure limit prescribed by the International Electrotechnical Commission (IEC), in IEC standard 62471 (IEC 62471:. Photobiological safety of lamps and lamp systems. (2006)).

### SUMMARY

At a high level, the present disclosure shows the success of 405 nm irradiation in inactivating SARS-CoV-2 and influenza A H1N1 viruses without the use of photosensitizers, supporting the possible use of 405 nm irradiation (and/or irradiation using closely-related wavelengths) as a tool to confer continuous decontamination of respiratory pathogens such as SARS-CoV-2 and influenza A viruses. The present disclosure further shows the increased susceptibility of lipid-enveloped viruses for irradiation in comparison to non-enveloped viruses, further characterizing the virucidal effects of visible light.

One aspect of the present disclosure provides a method of inactivating one or more lipid-enveloped viruses in an environment without an exogenous photosensitizer. The method includes providing light from at least one lighting element of a lighting device installed in the environment, the at least one lighting element configured to provide light toward a target area in the environment, the provided light having at least a virus-inactivating first component in a first range of wavelengths of 400 nanometers to 420 nanometers. The virus-inactivating first component of light produces an irradiance of at least 0.01 mW/cm² and not more than 1.0 mW/cm² as measured at a surface in the target area that is unshielded from the lighting device and located at a distance of 1.5 meters from an external-most luminous surface of the lighting device. Providing the light causes the one or more lipid-enveloped viruses to be inactivated, and the one or more lipid-enveloped viruses are inactivated without using the exogenous photosensitizer to cause inactivation of the one or more lipid-enveloped viruses.

Another aspect of the present disclosure provides a lighting system configured to inactivate one or more lipid-enveloped viruses in an environment without an exogenous photosensitizer. The lighting system includes a lighting device installed in the environment, the lighting device comprising at least one lighting element configured to provide light configured to provide light toward a target area in the environment, the provided light having at least a virus-inactivating first component in a first range of wavelengths of 400 nanometers to 420 nanometers. The virus-inactivating first component of light produces an irradiance of at least 0.035 mW/cm² and not more than 1.0 mW/cm² as measured at a surface in the target area that is unshielded from the lighting device and located at a distance of 1.5 meters from an external-most luminous surface of the lighting device, and the lighting system does not include an exogenous photosensitizer for causing inactivation of the one or more lipid-enveloped viruses, such that the providing of the light causes the one or more lipid-enveloped viruses to be inactivated without using the exogenous photosensitizer.

Still another aspect of the present disclosure provides a method of inactivating one or more lipid-enveloped viruses in an environment without an exogenous photosensitizer. The method includes providing light from at least one lighting element of a lighting device installed in the environment, the at least one lighting element configured to provide light toward a target area in the environment, the provided light having at least a virus-inactivating first component in a first range of wavelengths of 400 nanometers to 420 nanometers. The virus-inactivating first component of light produces an irradiance of at least 0.035 mW/cm² as measured at a surface in the target area that is unshielded from the lighting device and located at a distance of 1.5 meters from an external-most luminous surface of the lighting device. Providing the light causes the one or more lipid-enveloped viruses to be inactivated, and the one or more lipid-enveloped viruses are inactivated without using an exogenous photosensitizer to cause the inactivation of the one or more lipid-enveloped viruses.

### BRIEF DESCRIPTION OF THE DRAWINGS

The patent or application file contains at least one drawing executed in color. Copies of this patent or patent application publication with color drawing(s) will be provided by the United States Patent and Trademark Office upon request and payment of the necessary fee.

The accompanying figures, where like reference numerals refer to identical or functionally similar elements throughout the separate views, together with the detailed description below, are incorporated in and form part of the specification, and serve to further illustrate embodiments of concepts that include the claimed embodiments, and explain various principles and advantages of those embodiments.

FIG. 1 is an example graph showing normalized spectral power distribution for a lighting device showing peak irradiance at 405 nm;

FIG. 2A is a chart indicating time-dependent inactivation of SARS-CoV-2 in phosphate-buffered saline (PBS) by 405 nm irradiation at a dose of 0.035 mW/cm²;

FIG. 2B is a chart indicating time-dependent inactivation of SARS-CoV-2 in PBS by 405 nm irradiation at a dose of 0.076 mW/cm²;

FIG. 2C is a chart indicating time-dependent inactivation of SARS-CoV-2 in PBS by 405 nm irradiation at a dose of 0.15 mW/cm²;

FIG. 2D is a chart indicating time-dependent inactivation of SARS-CoV-2 in PBS by 405 nm irradiation at a dose of 0.6 mW/cm²;

FIG. 2E depicts a plaque phenotype comparison of treated and untreated SARS-CoV-2 samples;

FIG. 3A is a chart indicating time-dependent inactivation of influenza A virus (IAV) in PBS by 405 nm irradiation at a dose of 0.6 mW/cm²;

FIG. 3B depicts a plaque phenotype comparison of treated and untreated IAV sample;

FIG. 4A is a chart indicating time-dependent inactivation of encephalomyocarditis virus (EMCV) in PBS by 405 nm irradiation at a dose of 0.6 mW/cm²;

FIG. 4B depicts a plaque phenotype comparison of treated and untreated EMCV sample;

FIG. 5A is a chart comparing time-dependent inactivation of SARS-CoV-2, IAV, and EMCV at the studied doses of 405 nm irradiation, with the observed quantity of plaque-forming units (PFUs) represented on a linear percentage scale;

FIG. 5B is another chart comparing time-dependent inactivation of SARS-CoV-2, IAV, and EMCV at the studied doses of 405 nm irradiation, with the observed quantity of plaque-forming units (PFUs) represented on a logarithmic scale;

FIG. 6 is a schematic diagram of a lighting system constructed in accordance with the teachings of the present disclosure and employed in an environment susceptible to the transmission of pathogens;

FIG. 7 is a schematic of a portion of the environment of FIG. 6 including a lighting device constructed in accordance with the teachings of the present disclosure, the lighting device configured to inactivate pathogens in that portion of the environment;

FIG. 8A illustrates the CIE 1976 chromaticity diagram;

FIG. 8B is a close-up, partial view of the diagram of FIG. 8A, showing a range of curves of white visible light that can be output by the lighting device of FIG. 7 such that the lighting device can provide visually appealing, unobjectionable white light;

FIG. 9A is a plan view of one exemplary version of the lighting device of FIG. 7;

FIG. 9B is a rear perspective view of the lighting device of FIG. 9A;

FIG. 9C is a bottom view of the lighting device of FIGS. 9A and 9B, showing a first plurality of light-emitting elements configured to inactivate pathogens;

FIG. 9D is a partial, close-up view of a portion of the lighting device of FIG. 9C;

FIG. 10A is a perspective view of the lighting device of FIGS. 9A-9D installed in a receiving structure of the environment;

FIG. 10B is a cross-sectional view of FIG. 10A;

FIG. 11A is a bottom view of another exemplary version of the lighting device of FIG. 7, showing a second plurality of light-emitting elements configured to inactivate pathogens;

FIG. 11B is a partial, close-up view of a portion of the lighting device of FIG. 11A;

FIG. 12 illustrates another exemplary version of the lighting device of FIG. 7;

FIG. 13 illustrates another exemplary version of the lighting device of FIG. 7;

FIG. 14A is a perspective view of another exemplary version of the lighting device of FIG. 7;

FIG. 14B is a cross-sectional view of the lighting device of FIG. 14A;

FIG. 14C is another cross-sectional view of the lighting device of FIG. 14A, showing a first plurality of light-emitting elements configured to emit light that inactivate pathogens and a second plurality of light-emitting elements configured to emit light that blends with light emitted by the first plurality of light-emitting elements to produce a visually appealing visible light;

FIG. 14D is a block diagram of various electrical components of the lighting device of FIG. 14A;

FIG. 14E illustrates visually appealing white visible light that can be output by the lighting device of FIG. 14A when the environment is occupied;

FIG. 14F illustrates disinfecting light that can be output by the lighting device of FIG. 14A when the environment is not occupied;

FIG. 14G illustrates one example of how the lighting device of FIGS. 14A-14D can be controlled responsive to various dimming settings;

FIG. 15A is a perspective view of another exemplary version of the lighting device of FIG. 7;

FIG. 15B is similar to FIG. 15A, but with a lens of the lighting device removed so as to show a plurality of lighting elements;

FIG. 15C is a top view of FIG. 15B;

FIG. 15D is a close-up view of one of the plurality of lighting elements of FIGS. 15B and 15C;

FIG. 16A is a perspective view of another exemplary version of the lighting device of FIG. 7;

FIG. 16B is similar to FIG. 16A, but with a lens of the lighting device removed so as to show a plurality of lighting elements;

FIG. 16C is a top view of FIG. 16B;

FIG. 16D is a close-up view of one of the plurality of lighting elements of FIGS. 16B and 16C;

FIG. 17A is a perspective view of another exemplary version of the lighting device of FIG. 7;

FIG. 17B is a cross-sectional view of the lighting device of FIG. 17A;

FIG. 17C is another cross-sectional view of the lighting device of FIG. 17A, showing a first plurality of light-emitting elements configured to emit light that inactivate pathogens and a second plurality of light-emitting elements configured to emit light that also inactivate pathogens but blends with light emitted by the first plurality of light-emitting elements to produce a visually appealing visible light;

FIG. 18 is a schematic of a healthcare environment that includes a lighting device constructed in accordance with the teachings of the present disclosure and installed in a first room of the environment, and an HVAC unit that provides air to the first room and a second room in the healthcare environment;

FIG. 19A is a chart depicting the results of a study on a healthcare environment configured like the environment of FIG. 18, showing a bacterial reduction and a decrease in surgical site infections in the environment following installation of a lighting device constructed in accordance with the teachings of the present disclosure in the healthcare environment;

FIG. 19B graphically depicts the bacterial reduction listed in the chart of FIG. 19A;

FIG. 20A illustrates one example of a distribution of radiometric power by a lighting device constructed in accordance with the teachings of the present disclosure;

FIG. 20B illustrates a plot of one example of light distribution from a lighting device, constructed in accordance with the teachings of the present disclosure, as a function of the vertical angle from the horizontal;

FIG. 20C illustrates a plot of another example of light distribution from a lighting device, constructed in accordance with the teachings of the present disclosure, as a function of the vertical angle from the horizontal;

FIG. 20D illustrates a plot of another example of light distribution from a lighting device, constructed in accordance with the teachings of the present disclosure, as a function of the vertical angle from the horizontal;

FIG. 20E illustrates a plot of another example of light distribution from a lighting device, constructed in accordance with the teachings of the present disclosure, as a function of the vertical angle from the horizontal;

FIG. 20F depicts a chart of luminous flux for the light distribution plot of FIG. 20B;

FIG. 20G depicts a chart of luminous flux for the light distribution plot of FIG. 20C;

FIG. 20H depicts a chart of luminous flux for the light distribution plot of FIG. 20D;

FIG. 20I depicts a chart of luminous flux for the light distribution plot of FIG. 20E;

FIG. 21 is a flowchart of an exemplary method of providing doses of light sufficient to inactivate dangerous pathogens throughout a volumetric space over a period of time; and

FIG. 22 is a schematic diagram of an exemplary version of a control device constructed in accordance with the teachings of the present disclosure.

### DETAILED DESCRIPTION

As briefly discussed above, visible light within a wavelength range of 400-420 nm has been appreciated as a viable alternative to UVC irradiation in whole-room bacterial disinfection scenarios, particularly for MRSA, as irradiation using visible light within this wavelength range has been shown to reduce bacteria in occupied rooms and reductions in surgical site infections (Maclean, M. et al. Environmental decontamination of a hospital isolation room using high-intensity narrow-spectrum light. J. Hosp. Infect. 76, 247-251 (2010); Maclean, M., McKenzie, K., Anderson, J. G., Gettinby, G. & MacGregor, S. J. 405 nm light technology for the inactivation of pathogens and its potential role for environmental disinfection and infection control. J. Hosp. Infect. 88, 1-11 (2014); Murrell, L. J., Hamilton, E. K., Johnson, H. B. & Spencer, M. Influence of a visible-light continuous environmental disinfection system on microbial contamination and surgical site infections in an orthopedic operating room. Am. J. Infect Control 47, 804-810 (2019)). Although visible light having a wavelength of 405 nm and closely related wavelengths have been shown to be less germicidal than UVC light, the inactivation potential of such visible light (i.e., light having a wavelength of 405 nm and closely related wavelengths) has nonetheless been assessed and validated in pathogenic bacteria such as Listeria species (spp) and Clostridium spp, and in fungal species such as Saccharomyces spp and Candida spp ((Murrell, Hamilton, Johnson & Spencer, 2019; Maclean, M., Murdoch, L. E., MacGregor, S. J. & Anderson, J. G. Sporicidal effects of high-intensity 405 nm visible light on endospore-forming bacteria. Photochem. Photobiol. 89, 120-126 (2013); Murdoch, L., McKenzie, K., Maclean, M., Macgregor, S. & Anderson, J. Lethal effects of high-intensity violet 405-nm light on Saccharomyces cerevisiae, Candida albicans, and on dormant and germinating spores of Aspergillus niger. Fungal Biology 117, 519-527 (2013)).

It is thought that the underlying mechanism of visible light mediated inactivation of these bacterial and fungal species is associated with absorption of light via photosensitizers (e.g., porphyrins) found in the cells of the bacterial and fungal species, which results in the release of reactive oxygen species (ROS) (Dai, T. et al. Blue light for infectious diseases: Propionibacterium acnes, Helicobacter pylori, and beyond? Drug Resistance Updates 15, 223-236 (2012); Bumah, V. V. et al. Spectrally resolves infrared microscopy and chemometric tools to reveal the interaction between blue light (470 nm) and methicillin-resistant Staphylococcus aureus. Journal of Photochemistry and Photobiology B: Biology 167, 150-157 (2017)). The release of ROS causes direct damage to biomolecules such as proteins, lipids, and nucleic acids which are essential constituents of bacteria and fungi (and viruses). Further studies have shown that ROS can also lead to the loss of cell membrane permeability mediated by lipid oxidation (Hadi, J., Dunowska, M., Wu, S. & Brightweel, G. Control Measures for SARS-CoV-2: A Review on Light-Based Inactivation of Single-Stranded RNA Viruses. Pathogens 9, 737 (2020)). However, given that viruses lack endogenous photosensitizers (e.g., porphyrins in virions), efficient decontamination of viruses (both enveloped and non-enveloped) has been believed to require the addition of exogenous or external photosensitizers, e.g., dyes, external media, artificial saliva, blood, and feces (Tomb et al., 2017; Maclean, McKenzie, Anderson, Gettinby & MacGregor, 2014). When, for example, viruses are suspended in media containing endogenous and/or exogenous photosensitizers, 405 nm visible light has been demonstrated to inactivate viruses such as feline calicivirus (FCV), viral hemorrhagic septicemia virus (VHSV) and murine norovirus-1 (Tomb et al., 2017; Ho, D. T. et al. Effect of blue light emitting diode on viral hemorrhagic septicemia in olive flounder (Paralichthys olivaceus). Aquaculture 521, 735019 (2020); Wu, J. et al. Virucidal efficacy of treatment with photodynamically activated curcumin on murine norovirus bio-accumulated in oysters. Photodiagnosis and photodynamic therapy 12, 385-392 (2015)). Of note, most virus inactivation studies have been performed with the viruses suspended in media containing porphyrins, thus limiting the potential extent of use in broader settings.

Laboratory studies directed by the Applicant and described herein have, however, shown that systems and methods constructed in accordance with the present disclosure effectively and efficiently inactivate viruses such as SARS-CoV-2 and influenza A H1N1 by irradiating those viruses with 405 nm light (and/or similar wavelengths), supporting the possible use of irradiation using 405 nm light as a tool to confer continuous decontamination of respiratory pathogens such as SARS-CoV-2 and influenza A viruses. Of note, inactivation of these viruses by 405 nm irradiation using the systems and methods of the present disclosure does not require the virus to be accompanied by an exogenous photosensitizer (that is, a photosensitizer that is not inherent or “endogenous” to the virus itself), despite significant and long-standing evidence in the field suggesting the need for one or more external photosensitizers to inactivate viruses (barring the application of great doses of light substantially beyond the amounts described herein and in excess of the exposure limits of light as defined by IEC 62471, and beyond the realm of practicability in the example room disinfection scenarios described in the present disclosure).

It was previously speculated that lipid-enveloped viruses may be even less susceptible than non-enveloped viruses to inactivation by 405 nm light due to the presence of the lipid envelope surrounding the constituent parts of the virus. This lipid envelope, it was speculated, would act as a physical barrier which would shield the inner contents of the virus from 405 nm irradiation. Laboratory studies directed by the Applicant have shown that lipid-enveloped viruses and non-enveloped viruses respond differently to 405 nm light (and light having similar wavelengths). Surprisingly, however, those laboratory studies demonstrated that systems and methods constructed in accordance with the present disclosure are particularly effective at inactivating lipid-enveloped viruses, e.g., SARS-CoV-2 and influenza A viruses. It is believed that the 405 nm light is absorbed by the lipid envelope resulting in the release of reactive oxygen species (ROS), which oxidize the capsid or inner structure of the virus and thereby expose the genetic material of the virus.

The present disclosure describes various embodiments of lighting devices, lighting systems, and methods for inactivating viruses using 405 nm light (and light having similar wavelengths).

### 405 nm Light Exposure System

The studies described herein were conducted using the Indigo-Clean lighting fixture manufactured by Kenall Manufacturing. However, it will be appreciated and understood that other devices can equally be used, so long as the device has the same or similar characteristics/performance. Various examples of such products will be described in the present disclosure.

The form factor selected in the Indigo-Clean lighting fixture was a 6-inch downlight (M4DLIC6) to allow for use within a biosafety level 3 (BSL-3) rated containment hood for purposes of conducting the studies described herein. Within the hood, the distance between the face of the lighting fixture and the virus sample was 10 inches, which is much less than the normal 59 inches (1.5 meters (m)) used in normal, whole-room disinfection applications. The output of the fixture was modified electronically during its manufacture to match this difference in distances, and to ensure that the measurements would represent the performance of the device in actual use (e.g., in the standard 1.5 m whole-room disinfection applications). For the range of light output described in the studies, multiple discrete levels were created using pulse width modulation within an LED driver of the lighting fixture. These levels were made to be individually selectable using a simple knob on a control module attached to the lighting fixture.

As used herein, the term “dose” refers to the amount of visible light within the virus-inactivating 400-420 nm range (or a narrower or broader virus-inactivating range) delivered by the lighting fixture to the target organism. The dose is measured in milliwatts per square centimeter (mW/cm²), thus quantifying the dose in a manner similar to that used in ultraviolet (UV) light disinfection applications. The dose can also be referred to herein as an “irradiance” produced by the 400-420 nm light at a location (e.g., surface) treated by the virus-inactivating light. To fully examine the effect of virus-inactivating light, a range of irradiance values were used in the studies, the values representing actual product deployment conditions in occupied rooms. The lowest irradiance value (0.035 mW/cm²) represents a single-mode, lower wattage used in general lighting applications, while the highest value (0.6 mW/cm²) represents a dual-mode, high wattage used in critical care applications such as an operating room.

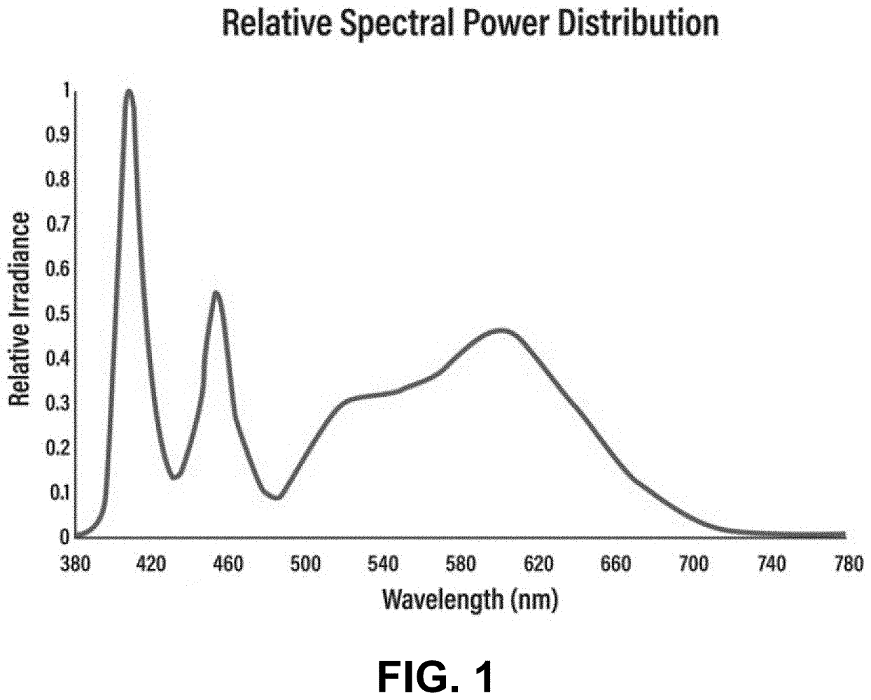

The lighting fixture in the studies described herein was placed in a rig to ensure a consistent distance of 10 inches between the fixture and the virus samples. The light output of the fixture in the test rig was measured using a Stellar-RAD Radiometer from StellarNet, configured to make wavelength and irradiance measurements from 350 to 1100 nm with <1 nm spectral bandwidth using a NIST-traceable calibration. To ensure that a regular white light portion of the light emitted by the lighting fixture (which is non-disinfecting) was not measured, the irradiance measurement was electronically linked to a 1 nm bandwidth over the 400-420 nm range. A normalized spectral power distribution profile for the Indigo-Clean M4DLIC6 is shown in FIG. 1, with the profile showing a peak irradiance at approximately 405 nm. The absolute value of the irradiance measurement was determined using a NIST-traceable calibration, as previously described. Accordingly, as used in the following sections describing the virus-inactivating studies, references to a dose of 405 nm light (e.g., “405 nm light at 0.035 mW/cm²) refers more specifically to a dose of virus-inactivating light in the 400-420 nm range with a peak irradiance of approximately 405 nm.

### Cells and Viruses

Vero-E6 cells (ATCC® ^{CRL}1586™, clone E6) were maintained in Dulbecco's Modified Eagle Medium (DMEM) complimented with 10% heat-inactivated Fetal Bovine Serum (HI-FBS; PEAK serum), penicillin-streptomycin (Gibco; 15140-122), HEPES buffer (Gibco; 15630-080) and MEM non-essential amino-acids (Gibco; 25025CL) at 37° C. with 5% CO₂. Vero-CCL81 (ATCC® CRL-81™) cells and MDCK cells (ATCC® CRL-34) were cultured in DMEM supplemented with 10% HI-FBS and penicillin-streptomycin at 37° C. with 5% CO₂. All experiments involving SARS-CoV2 (USA-WA1/202, BEI resource—NR52281) were conducted within a BSL3 containment facility at Icahn School of Medicine at Mount Sinai by trained workers upon authorization of protocols by a biosafety committee. Amplification of SARS-CoV-2 viral stocks was done in Vero-E6 cell confluent monolayers by using an infection medium composed of DMEM supplemented with 2% HI-FBS, non-essential amino acids (NEAA), HEPES and penicillin-streptomycin at 37° C. with 5% CO₂ for 72 hours. Influenza A virus (IAV) used in the studies was generated using plasmid-based reverse genetics system (Martinez-Sobrido, L. & Garcia-Sastre, a. Generation of recombinant influenza virus from plasmid DNA. J. Vis. Exp. (42). pii: 2057. doi, 10.3791/2057 (2010)). The viral backbone used in the studies was A/Puerto Rico/8/34/Mount Sinai (H1N1) under the GenBank accession number AF389122. IAV-PR8 virus was grown and titrated in MDCK as previously described (Ibid.). As a non-enveloped virus, the cell culture adapted murine encephalomyocarditis virus (EMCV; ATCC® VR-12B) was propagated and titrated in Vero-CCL81 cells with DMEM and 2% HI-FBS and penicillin-streptomycin at 37° C. with 5% CO₂ for 48 hours (Carocci, M. & Bakkali-Kassimi, L. The encephalomyocarditis virus. Virulence 3, 351-367 (2012)).

### Virus Inactivation and Plaque Assay Methodologies

The SARS-CoV-2 virus was exclusively handled at the Icahn School of Medicine BSL-3 facility, and studies involving IAV and EMCV were handled in BSL-2 conditions. Indicated plaque-forming unit (PFU) amounts were mixed with sterile 1× PBS and were irradiated in 96 well formal cell culture plates in triplicates. In these studies, a starting amount of 5×10⁵ PFU for SARS-CoV-2 and starting amounts of 1×10⁵ PFU for IAV and EMCV were used. The final volume for inactivation were 250 microliters (μL) per replicate. The untreated samples were prepared the same way and were left inside the biosafety cabinet isolated from the inactivation device at room temperature.

The plates were sealed with qPCR plate transparent seal, and an approximate 10% reduction of the intensity was observed due to the sealing film. The distance from the fixture lamp and the samples was measured to be 10 inches. All samples were extracted at times as indicated below, frozen at -80° C., and thawed together for titration via plaque assays.

Confluent monolayers of Vero-E6 cells in 12-well plate format were infected with 10-fold serially diluted samples in 1× PBS supplemented with bovine serum albumin (BSA) and penicillin-streptomycin for one hour, while the plates were gently shaken every 15 minutes. Afterwards, the inoculum was removed, and the cells were incubated with an overlay composed of MEM with 2% FBS and 0.05% Oxoid agar for 72 hours at 37° C. with 5% CO₂. The plates were subsequently fixed using 10% formaldehyde overnight, and the formaldehyde was removed along with the overlay. Fixed monolayers were blocked with 5% milk in Tris-buffered saline with 0.1% tween-20 (TBS-T) for one hour. Afterwards, plates were immunostained using a monoclonal antibody against SARS-CoV-2 nucleoprotein (Creative-Biolabs; NP1C7C7) at a dilution of 1:1000, followed by 1:5000 anti-mouse IgG monoclonal antibody, and were developed using KPL TrueBlue peroxidase substrate for 10 minutes (Seracare; 5510-0030). After washing of the plates with distilled water, the number of plaques on the plates were counted.

The plaque assays for IAV and EMCV were performed in a similar fashion. The IAV plaque assays used confluent monolayers of MDCK cells supplemented with MEM-based overlay with TPCK-treated trypsin. For EMCV, Vero-CCL81 cells were used to perform plaque assays in 6 well plate format. Plaques for IAV and EMCV were visualized using crystal violet.

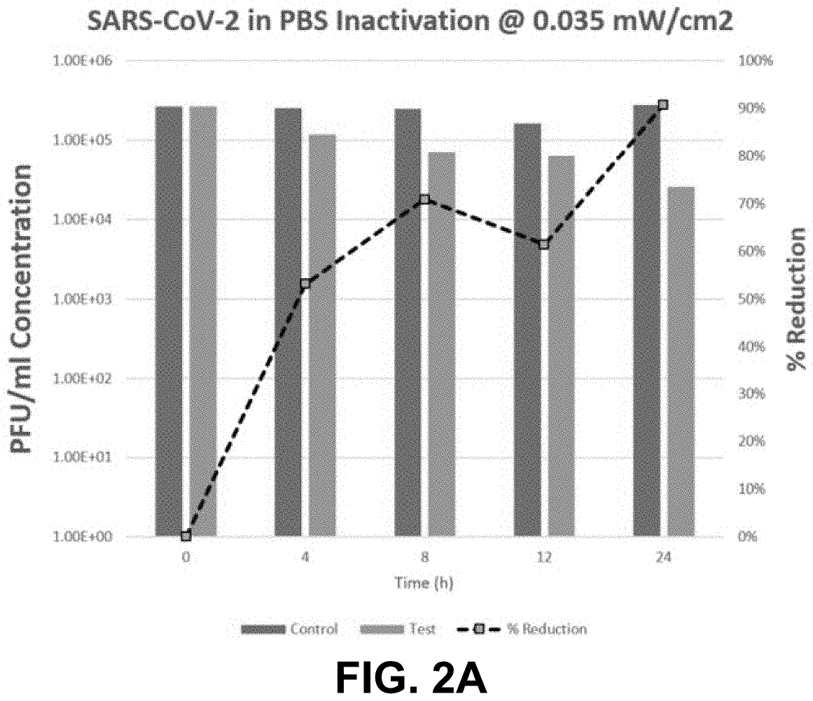

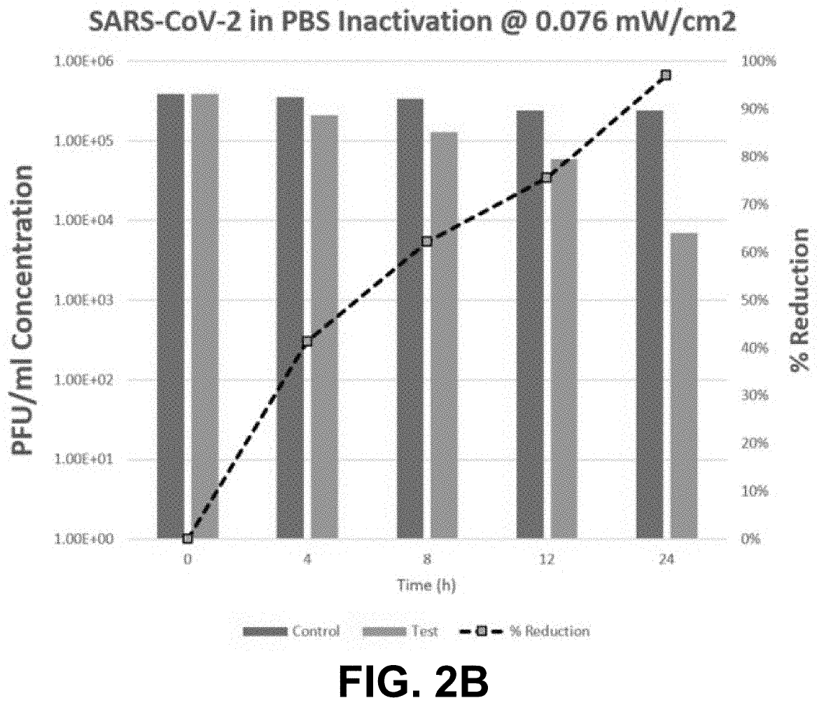

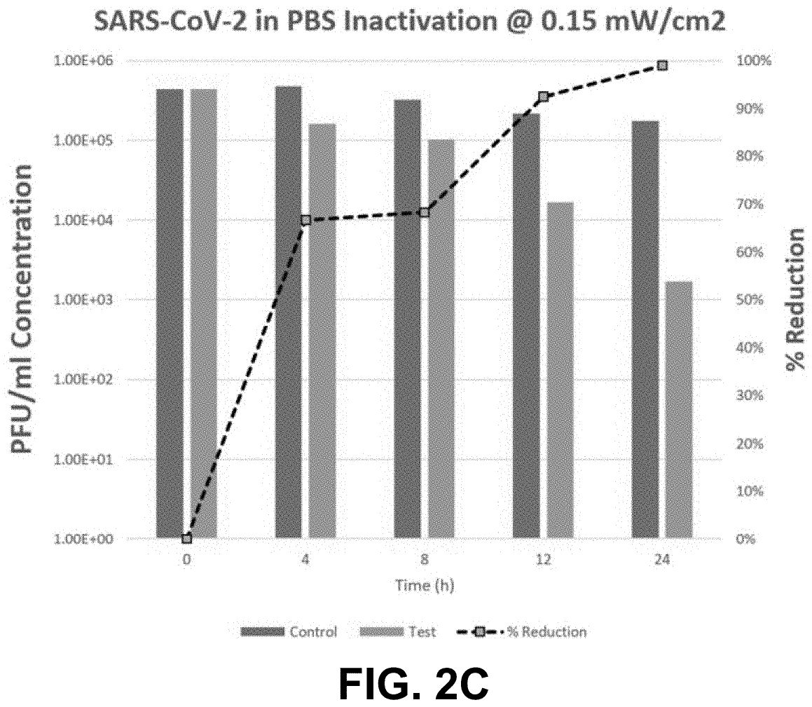

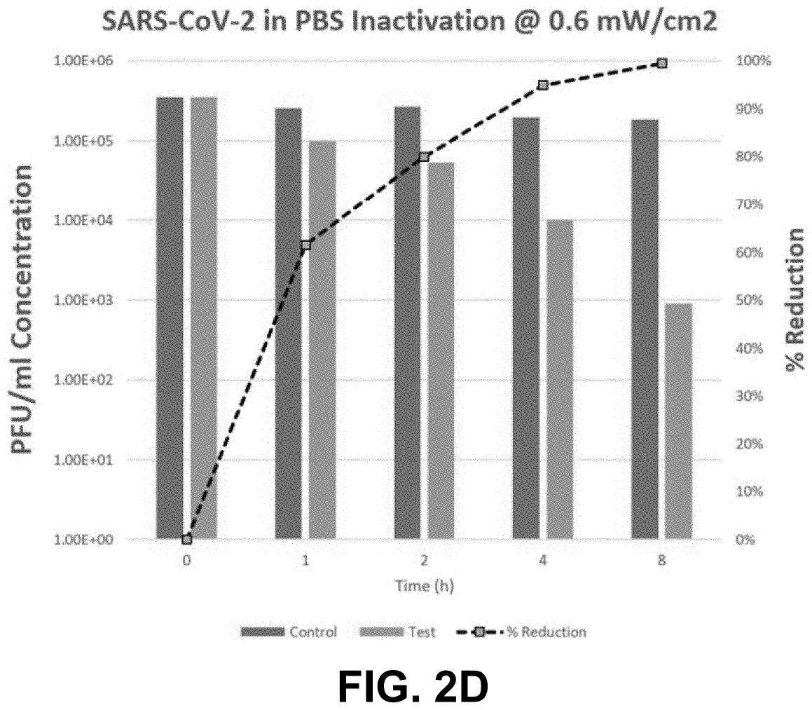

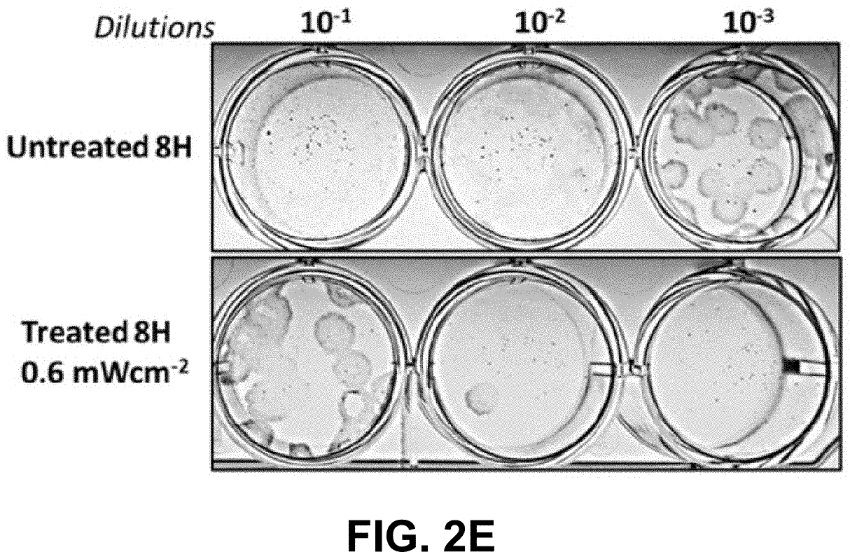

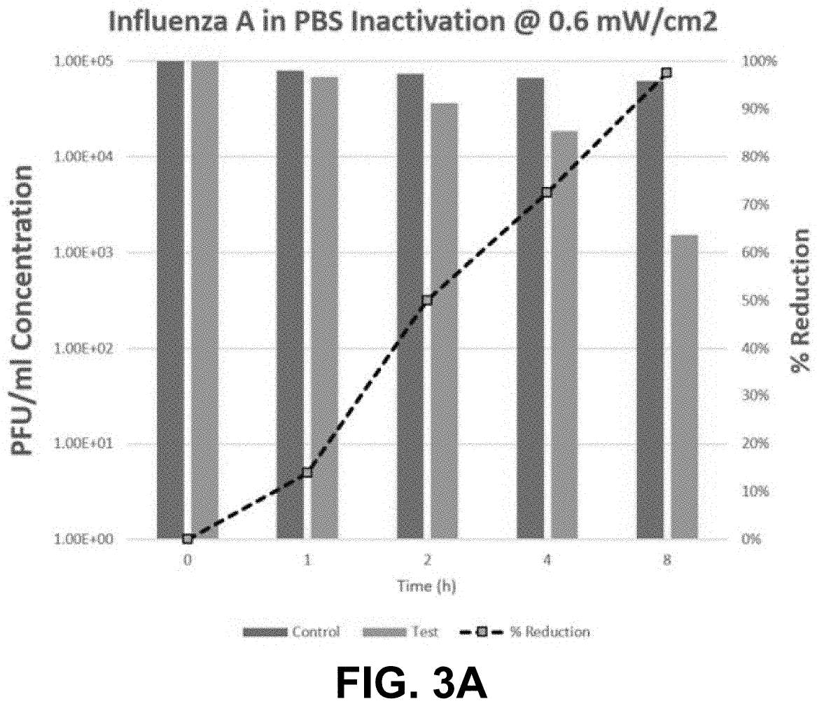

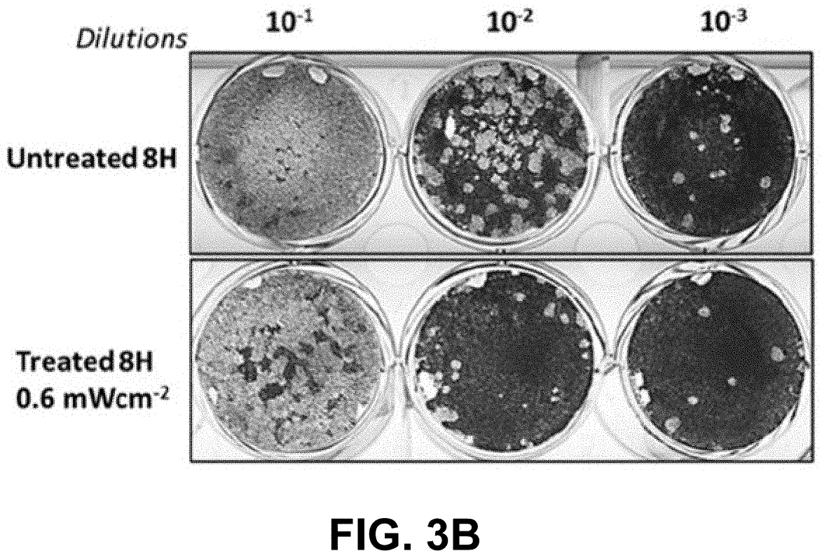

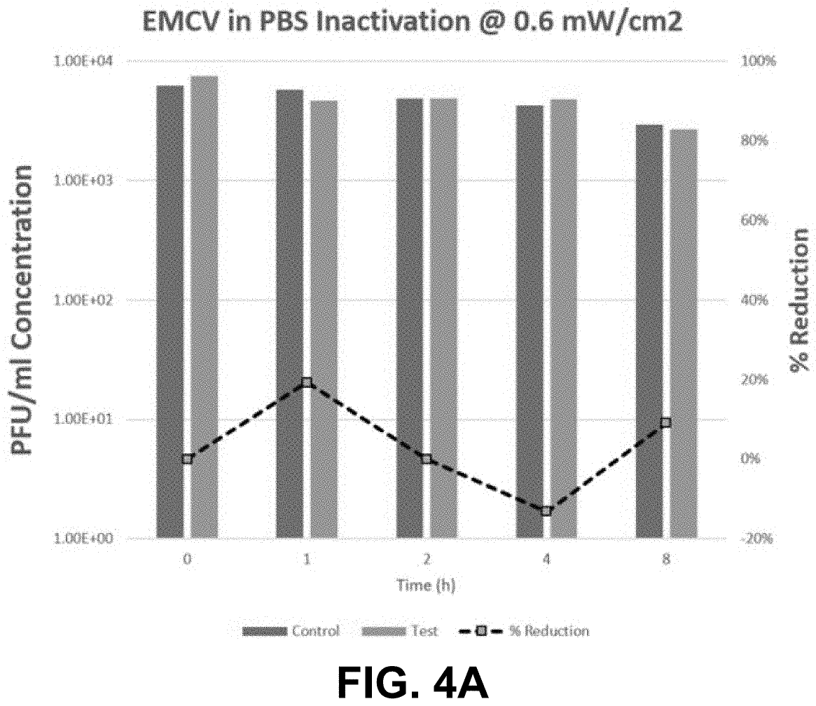

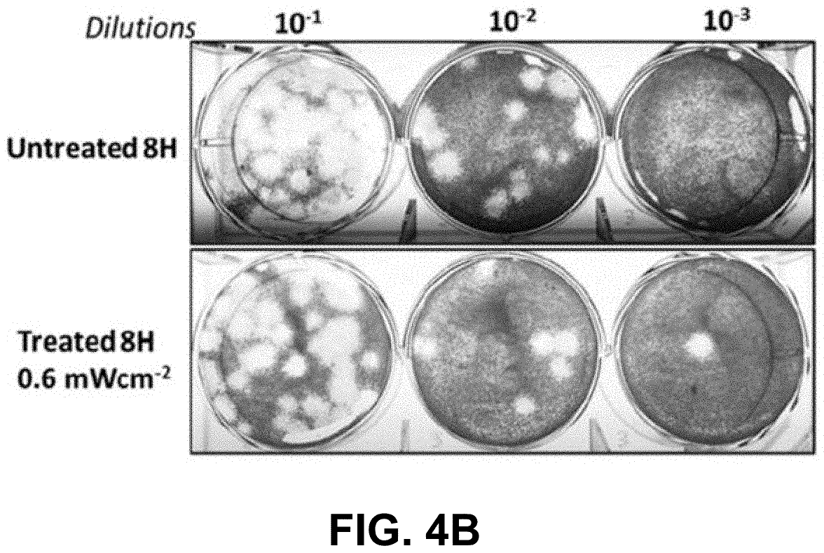

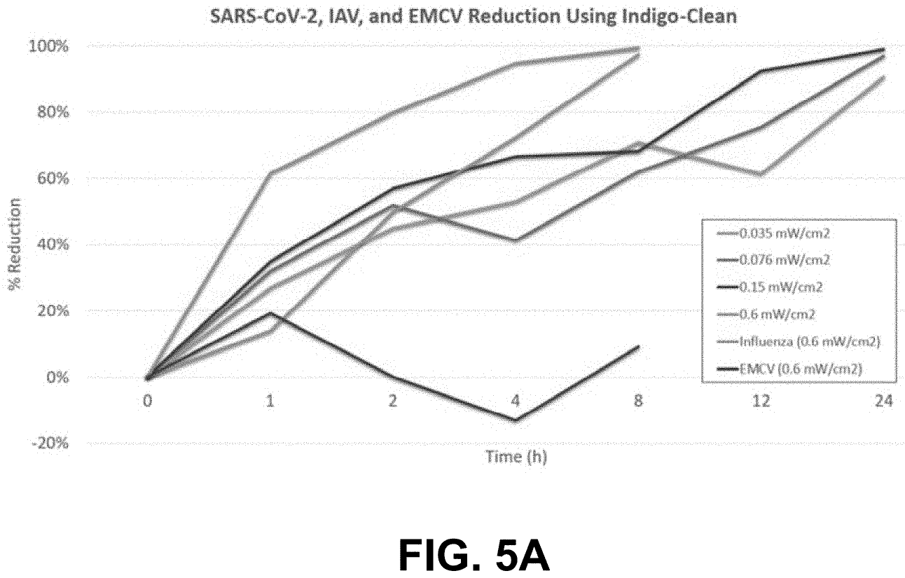

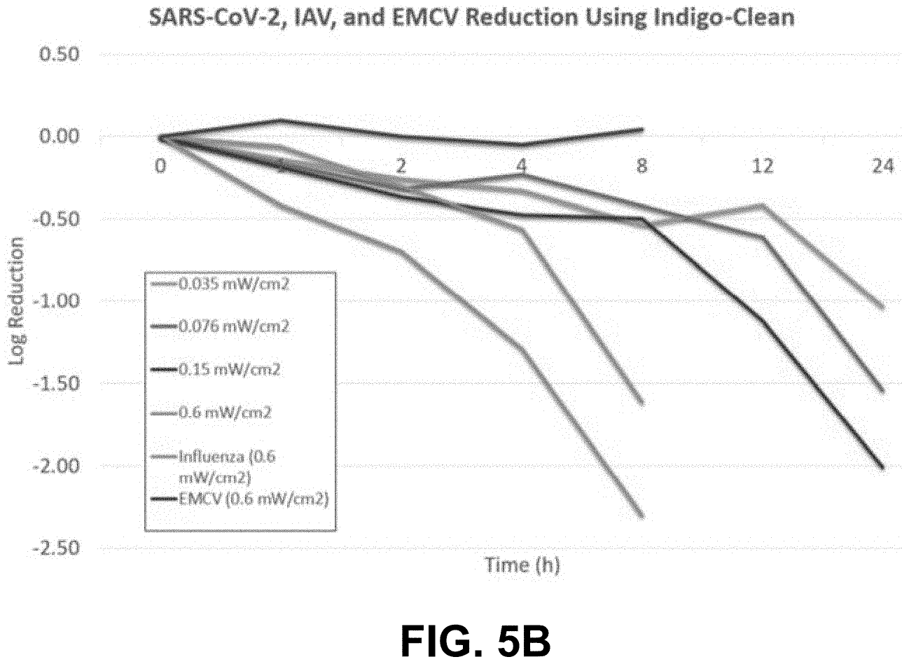

Subsequent sections of this detailed description will describe the resulting data as obtained via plaque assays. The data will be described with respect to FIGS. 2A-2E, 3A, 3B, 4A, 4B, 5A, and 5B, which depict charts and plaque phenotype comparisons indicative of the virus inactivation in PBS achieved via application of 405 nm light at various doses. More particularly, FIGS. 2A-2D chart time-dependent inactivation of SARS-CoV-2 using different doses, and FIG. 2E depicts the plaque phenotype comparison of treated (i.e., irradiated) and corresponding untreated SARS-CoV-2 samples. FIG. 3A charts time-dependent inactivation of IAV, with FIG. 3B depicting the plaque phenotype comparison of treated and corresponding untreated IAV samples. FIG. 4A charts time-dependent inactivation of EMCV, with FIG. 4B depicting the plaque phenotype comparison of treated and corresponding untreated EMCV samples. FIGS. 5A and 5B chart a comparison of the inactivation achieved in the irradiated SARS-CoV-2, IAV, and EMCV samples over the monitored durations of time.

In the charts of FIGS. 2A-2D, 3A, and 4A, orange bars indicate viral titer of virus samples treated with the indicated irradiation dose in the absence of photosensitizers, as measured over a number of hours (h) of irradiation. Blue bars indicate viral titer of corresponding untreated samples that were left in the biosafety cabinet under the same conditions for the same length of time, but not subjected to irradiation. The viral titer amounts depicted in the charts are measured in PFU/ml, and charted on a logarithmic scale. Each of the six combined charts of FIGS. 2A-2D, 3A, and 4A further includes a “reduction curve,” each point on the curve being the amount of reduction of viral titer in the treated sample (orange bar) compared to the corresponding untreated sample (blue bar) for the same duration of irradiation. FIG. 5A plots these six reduction curves on one chart for ease of comparison, and FIG. 5B plots the same reduction data in terms of logarithmic reduction at each point on the respective logarithmic reduction curves (i.e., logarithmic reduction of viral titer in each treated sample compared to the corresponding untreated sample at each respective time marker). By still another form of measurement, as will be provided herein, the reduction in a sample over the initial viral titer can be measured at any time marker (e.g., one hour, four hours, 12 hours, etc.) by comparing the viral titer at the time marker against the initial viral titer at zero hours, this initial viral titer being represented herein as t₀. In any case, measurement of viral titer as described herein were performed in independent triplicates, and by obtaining the viral titer values as the average of the three measured values.

### Results of Irradiation (SARS-CoV-2)

FIGS. 2A-2D chart dose- and time-dependent inactivation of SARS-CoV-2 viruses in PBS by 405 nm irradiation (i.e., light in the 400-420 nm range with a peak at 405 nm as produced by the lighting fixture described in the foregoing). Specifically, FIGS. 2A-2D chart inactivation from irradiation at a dose of 0.035 mW/cm² (FIG. 2A), a dose of 0.076 mW/cm² (FIG. 2B), a dose of 0.15 mW/cm² (FIG. 2C), and a dose of 0.6 mW/cm² (FIG. 2D). The inactivation via 405 nm light at the 0.035, 0.076, and 0.15 mW/cm² doses was measured by determining viral reduction over a duration of 24 hours with sampling at four, eight, 12, and 24 hours. The inactivation at the 0.6 mW/cm² dose was measured over a duration of eight hours with sampling at one, two, four, and eight hours. FIG. 2E depicts a plaque phenotype comparison from the irradiation dose of 0.6 mW/cm², specifically comparing treated (i.e., irradiated) and untreated SARS-CoV-2 virus samples at three different dilutions after eight hours. Fixed and blocked plaques were immunostained using an anti-SARS-CoV-2/NP antibody before being developed using TrueBlue reagent.

For the lowest irradiation dose of 0.035 mW/cm² applied to SARS-CoV-2, as charted in FIG. 2A, a reduction of 53.1% was observed in comparison to the corresponding untreated sample after four hours (a 0.33 log₁₀ reduction, and a 55.08% reduction from the initial viral titer (T₀) in the virus sample). After eight hours, a 70.9% (0.54 log₁₀) reduction was observed, and after 12 hours, a 61.4% reduction (0.41 log₁₀) was observed, compared to the corresponding untreated sample at the same respective time markers. Finally, after 24 hours of irradiation, a reduction of 90.7% (1.03 log₁₀) was observed (corresponding to a reduction of 90.17% or approximately 10-times reduction from T₀).

With a slightly higher dose of 0.076 mW/cm², as charted in FIG. 2B, a reduction of 41.4% (0.23 log₁₀) was observed after four hours. After eight hours, a 62.1% (0.42 log₁₀) reduction was observed, and after 12 hours, a 75.6% reduction (0.61 log₁₀) was observed, compared to the corresponding untreated sample at the same respective time markers. Finally, after 24 hours of irradiation at 0.076 mW/cm², a reduction of 97.1% (1.54 log₁₀) was observed (or a reduction of 98.22% or 56-times reduction compared to T₀ in the 0.076 mW/cm² study).

Increasing the irradiation dose to 0.15 mW/cm², as charted in FIG. 2C, resulted in an observed reduction of 66.7% (0.48 log₁₀) after four hours, which increased to 68.3% (0.50 log₁₀) after eight hours. After 12 hours, a reduction of 92.4% (1.12 log₁₀) was observed. At the last measurement after 24 hours, a total reduction of 99.0% (2.01 log₁₀) was observed (corresponding to a 99.61% or 256-times reduction from T₀ in the 0.15 mW/cm² study).

The final SARS-CoV-2 experiment, as charted in FIG. 2D, used a still-higher irradiation dose of 0.6 mW/cm² over a shorter time frame of eight hours. After one hour, a reduction of 61.5% (0.41 log₁₀) was observed, which increased to 80.0% (0.70 log₁₀) at two hours. After four hours, a reduction of 94.9% (0.48 log₁₀) was observed (a 97.15% reduction from T₀). Finally, after total eight hours of irradiation at 0.6 mW/cm², a reduction of 99.5% (2.30 log₁₀) was observed (corresponding to a 99.74% or 385-times reduction from T₀). The plaque phenotype comparison as depicted in FIG. 2E reflects the reduction in viral titer after eight hours of irradiation at 0.6 mW/cm², compared to the corresponding untreated sample.

### Results of Irradiation (IAV)

In view of the observations derived from applying the 405 nm light to the lipid-enveloped SARS-CoV-2 virus, the separate inactivation study of a different lipid-enveloped virus was conducted using influenza A Puerto Rico (A/H1N1/PR8-Mount Sinai) virus strain. FIG. 3A charts time-dependent inactivation of influenza A virus (IAV) in PBS by 405 nm irradiation at a dose of 0.6 mW/cm², the inactivation being measured over a duration of eight hours with sampling at one, two, four, and eight hours. FIG. 3B depicts a plaque phenotype comparison from the irradiation dose of 0.6 mW/cm², comparing treated (irradiated) and untreated IAV virus samples at three different dilutions after eight hours. Fixed and blocked plaques were stained using crystal violet.

As charted in FIG. 3A, 405 nm irradiation with the highest dose of 0.6 mW/cm² resulted in a reduction of 13.9% (0.06 log₁₀) compared to the corresponding untreated sample at one hour (or a reduction of 31.11% from T₀). After two hours, though, a reduction of 50.0% (0.30 log₁₀) was observed with reference to the untreated sample at two hours (or a 63.33% reduction from T₀). After four hours, a 72.5% (0.56 log₁₀) reduction was observed (or a 81.56% reduction from T₀). Finally, after eight hours of irradiation at 0.6 mW/cm², a reduction of 97.6% (1.61 log₁₀) was observed (corresponding to a 98.49% or 66-times reduction from T₀ in this study). The stability of IAV in PBS at room temperature for a duration of eight hours was demonstrated by way of the negligible reduction of viral titer in the corresponding untreated sample. The plaque phenotype comparison as depicted in FIG. 3B reflects the reduction in viral titer after eight hours of irradiation, compared to the corresponding untreated sample.

### Results of Irradiation (EMCV)

In view of the successful inactivation of the lipid-enveloped SARS-CoV-2 and IAV viruses in PBS by 405 nm irradiation, a non-enveloped RNA virus chosen for experimentation was encephalomyocarditis virus (EMCV), which is derived from the Picornaviridae family. For experimentation with EMCV, EMCV in PBS was irradiated at the dose of 0.6 mW/cm² for a duration of 8 hours, in a manner similar to that described with respect to SARS-CoV-2 and IAV.

FIG. 4A charts time-dependent inactivation of EMCV in PBS by 405 nm irradiation at the 0.6 mW/cm² dose, with the inactivation being measured over a duration of eight hours with sampling at one, two, four, and eight hours. FIG. 4B depicts a plaque phenotype comparison from the irradiation dose of 0.6 mW/cm², specifically comparing treated (irradiated) and untreated EMCV virus samples at three different dilutions after eight hours. Fixed and blocked plaques were stained using crystal violet. FIGS. 4A and 4B illustrate that EMCV in PBS shows reduced susceptibility to 405 nm irradiation, in contrast to the lipid-enveloped RNA viruses SARS-CoV-2 and IAV. Specifically, as charted in FIG. 4A, only a 9.1% (0.04 log₁₀) reduction was achieved compared to the corresponding untreated sample at eight hours (or, a 57.14% or two-times reduction from the initial viral titer T₀ for the EMCV study). Thus, the plaque reduction at eight hours did not indicate the same dramatic reduction as observed with the SARS-CoV-2 and IAV studies.

FIGS. 5A and 5B chart the reduction curves resulting from each of the SARs-CoV-2, IAV, and EMCV studies at their respective irradiation doses over the monitored durations of time. FIG. 5A plots the reduction curves in terms of percentage reduction, illustrating the increasing success of higher doses of 405 nm irradiation in inactivating SARS-CoV-2. The reduction curves further show the still-significant success of 405 nm irradiation at 0.6 mW/cm² in inactivating IAV, and the lack of substantial effect of 405 nm irradiation at 0.6 mW/cm² in inactivating EMCV. Charting the reduction curves in terms of logarithmic curve in FIG. 5B similarly illustrates these effects, with 405 nm inactivating significant amounts of SARS-CoV-2 and IAV viruses but not producing substantial effect in inactivating EMCV.

### Further Discussion of 405 nm Irradiation Results

The studies described herein thus confirmed the positive impact of 405 nm enriched visible light technology in terms of inactivating respiratory pathogens such as SARS-CoV-2 and IAV. The ongoing SARS-CoV-2 pandemic has affected day-to-day functions in the entire world, raising concerns not only with regards to therapeutics but also in the context of virus survivorship and decontamination (Derraik, J. G., Anderson, W. A., Connelly, E. A. & Anderson, Y. C. Rapid evidence summary on SARS-CoV-2 survivorship and disinfection, and a reusable PPE protocol using a double-hit process. medRxiv (2020)). Taking into consideration the rapid spread of SARS-CoV-2 from person to person by droplets, aerosols, and fomites, whole-room disinfection systems that utilize 405 nm enriched visible light technology can therefore be viewed as a significant supplement to best practices for interrupting transmission of the SARS-CoV-2 virus in an environment. Importantly, these types of disinfection systems can operate continuously, as 405 nm visible light is considered to be safe for humans based upon the exposure guidelines defined by the International Electrotechnical Commission (IEC) 62471 standard. Thus, once this disinfection has been in use for a period of time, the environment will be cleaner and safer the next time it is occupied by humans.

More particularly, the studies described herein confirmed that 405 nm enriched visible light technology inactivates respiratory pathogens such as SARS-CoV2 and IAV even without the use of any exogenous photosensitizers in or on those pathogens. Indeed, the studies described herein showed that irradiation with low intensity of 0.035 mW/cm² visible 405 nm light yielded a 53.1% reduction from the corresponding untreated sample (and 55.08% reduction from T₀) of SARS-CoV-2 after four hours, and a total of 90.7% reduction from the corresponding untreated sample (90.17% reduction from T₀) after 24 hours. A slightly higher dose of 0.076 mW/cm2 resulted in a 97.1% reduction from the corresponding untreated sample (98.22% reduction from T₀) after 24 hours, while a dose of 0.15 mW/cm² resulted in 66.7% reduction from the corresponding untreated sample (63.64% reduction from T₀) after four hours and 99.0% reduction (99.61% reduction from Tₒ) after 24 hours of irradiation. Finally, increasing the dose to 0.6 mW/cm2 yielded 99.5% reduction in viral titer from the corresponding untreated sample (99.74% reduction from T₀) after eight hours, indicating both a time-dependent and dose-dependent inactivation of infectious viruses. The studies described in the foregoing selected conventional plaque assays as the read out to specifically estimate infectious virus titers upon disinfection. Alternate methods based in the quantification of viral RNA via PCR techniques might be misleading, as such methods detect viral RNA from both infectious and noninfectious virions.

SARS-CoV-2 is a lipid-enveloped virus composed of an ssRNA genome, and the data described in the foregoing confirm that the virus is susceptible to visible light-mediated inactivation. To further confirm these observations, similar studies were repeated using influenza A virus (IAV), which, like SARS-CoV-2, is a human respiratory virus with a lipid envelope and an RNA genome. Upon irradiation for one hour at 0.6 mW/cm², a reduction of 13.9% compared to the corresponding untreated sample (31.11% reduction from T₀) was observed, compared to the reduction of 61.5% (71.52% reduction from T₀) for SARS-CoV-2 under the same conditions for the same duration of time. While both the SARS-CoV-2 and IAV viruses have lipid envelopes, this difference in results is clear and merits further study. One possible explanation of the difference in results is the virion size for IAV creating a physically smaller cross-section for light absorption (IAV ˜120 nm and SARS-CoV-2 -200 nm) (Bouvier, N. M. & Palese, P. The biology of influenza viruses. Vaccine 26, D49-D53 (2008); Bar-On, Y. M., Flamholz, A., Phillips, R. & Milo, R. Science Forum: SARS-CoV-2 (COVID-19) by the numbers. Elife 9, e57309 (2020)). Nevertheless, both viruses were largely inactivated after eight hours, with 97.6% reduction of viral titer from the corresponding untreated sample (98.49% reduction from T₀) for IAV after eight hours of irradiation at 0.6 mW/cm², and 99.5% reduction from the corresponding untreated sample (99.74% reduction from T₀) for SARS-CoV-2 under the same conditions. Intriguingly, it was observed that both RNA viruses were able to remain stable in phosphate-buffered saline (PBS) at room temperature for at least 24 hours, indicating minimal decay which is consistent with previous studies (Derraik, Anderson, Connelly & Anderson, 2020; Wang, X., Zoueva, O., Zhao, J., Ye, Z. & Hewlett, I. Stability and infectivity of novel pandemic influenza A (H1 N1) virus in blood-derived matrices under different storage conditions. BMC infectious diseases 11, 1-6 (2011)).

The previous results in the field for irradiating non-enveloped viruses such as EMCV with visible light demonstrated the need for external photosensitizers, such as artificial saliva, blood, feces, etc. (Tomb et al., 2017; Derraik, Anderson, Connelly & Anderson, 2020). Accordingly, these previous results would suggest that, without a porphyrin-containing medium, little to no activation of EMCV would occur upon irradiation with visible 405 nm visible light. Indeed, the studies described herein confirmed that EMCV (in PBS) is generally not susceptible to inactivation by 405 nm irradiation. For example, using the irradiance dose of 0.6 mW/cm², only a minimal level of reduction was observed after eight hours (around 9.1% reduction of viral titer from the corresponding untreated sample). Indeed, this reduction appears to be consistent with the statistical precision of reductions measured from shorter irradiation durations of one, two, and four hours, and moreover, the reduction in the treated sample after eight hours still did not differ significantly from the corresponding eight-hour control sample. For comparison, a study involving the M13-bacteriophage virus (another non-enveloped virus) showed a 3-Log reduction by applying 425 nm visible light with an irradiance of 50 mW/cm² for 10 hours. Given that the applied irradiance in this study is almost 100 times greater than the highest 0.6 mW/cm² irradiance used in the studies described in the foregoing, it is believed that non-enveloped viruses such as EMCV may require much higher doses of visible light for inactivation (Tomb, R. M. et al. Inactivation of Streptomyces phage ΦC31 by 405 nm light: Requirement for exogenous photosensitizers? Bacteriophage 4, e32129 (2014)).

The studies described in the foregoing used a neutral liquid media composed of PBS without any photosensitizers, and showed that visible light can indeed inactivate lipid-enveloped viruses, differing from the prevailing theory in the field that photosensitizers (exogenous or endogenous) are a requirement for inactivation. Other studies which used visible light-based irradiation produced theories involving the role of light as an inactivation mechanism, but not specifically involving 405 nm irradiation (Maclean, McKenzie, Anderson, Gettinby & MacGregor, 2014; Maclean, Murdoch, MacGregor & Anderson, 2013; Tomb et al., 2017). A first theory proposed that small amounts of 420-430 nm light emitted from the source contributes to the viral inactivation (Richardson, T. B. & Porter, C. D. Inactivation of murine leukemia virus by exposure to visible light. Virology 341, 321-329 (2005)). This theory most likely does not apply to the studies described in the foregoing, as the spectrum of light used in the present studies contained very little irradiance at these wavelengths (FIG. 1). A second theory proposed the utilization of UV-A light (390 nm) for visible light-based irradiation. 390 nm UV-A wavelength is known to create oxidative stress upon viral capsids, but the primary mechanism of action of UV-A inactivation of breaking down of pathogen DNA is considerably different from that observed from the studies described herein (Girard, P. et al. UVA-induced damage to DNA and proteins: direct versus indirect photochemical processes (Journal of Physics: Conference Series Ser. 261, IOP Publishing, 2011)). Thus, further experimentation in this area in view of this second theory would likely have focused more particularly on lower wavelengths of UV light (e.g., 370 nm), particularly in view of the studies by Tomb et. al which, as discussed above, casted doubt on 405 nm irradiation itself as a safe and practical virus inactivation mechanism due to the excessive amounts of 405 nm light required (e.g., amounts in excess of the limits prescribed by IEC 62471).

One potential limitation of the present studies is that the inactivation assays were performed in static liquid media, as opposed to aerosolized droplets. While the use of visible light in air disinfection has been briefly studied and shown to increase effectiveness approximately four-fold (Dougall, L. R., Anderson, J. G., Timoshkin, I. V., MacGregor, S. J. & Maclean, M. Efficacy of antimicrobial 405 nm blue-light for inactivation of airborne bacteria (Light-Based Diagnosis and Treatment of Infectious Diseases Ser. 10479, International Society for Optics and Photonics, 2018)), further studies involving dynamic aerosolization are merited to better understand the fuller potential of visible light-mediated viral inactivation. Nonetheless, the studies described in the foregoing show the increased susceptibility of enveloped respiratory viral pathogens to 405 nm light-mediated inactivation in the absence of photosensitizers. Moreover, the irradiances used in these studies were very low, and may be easily applied to safely and continuously disinfect occupied areas within hospitals, schools, restaurants, offices, and/or other environments.

Further information regarding the studies described herein can be found in Rathnasinghe, R., Jangra, S., Miorin, L. et al. The virucidal effects of 405 nm visible light on SARS-CoV-2 and influenza A virus. Sci. Rep. 11, 19470 (2021), which is hereby incorporated by reference in its entirety.

Subsequent portions of this detailed description will provide various examples of lighting devices, lighting systems, and methods for inactivating viruses via 405 nm visible light or similar wavelengths (e.g., 400-420 nm light with peak irradiance at about 405 nm) consistent with the studies described above. It should be understood that still other modifications may be possible, including via combination with devices and/or methods described in the foregoing sections of the present disclosure.

### Example Lighting Systems and Methods

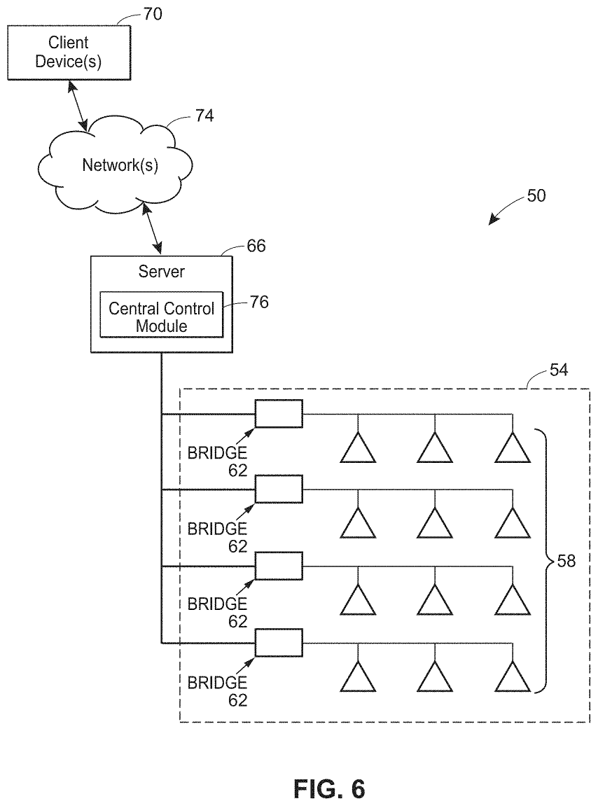

FIG. 6 depicts a lighting system 50 that may be implemented or included in an environment 54, such as, for example, a hospital, a doctor's office, an examination room, a laboratory, a nursing home, a health club, a retail store (e.g., grocery store), a restaurant, or other space or building, or portions thereof, where it is desirable to both provide illumination and to reduce, and ideally eliminate, the existence and spread of the pathogens described above.

The lighting system 50 illustrated in FIG. 6 generally includes a plurality of lighting devices 58, a plurality of bridge devices 62, a server 66, and one or more client devices 70 configured to connect to the server 66 via one or more networks 74. Of course, if desired, the lighting system 50 can include more or less components and/or different components. For example, the lighting system 50 need not necessarily include bridge devices 62 and/or client devices 70.

Each of the lighting devices 58 is installed in or at the environment 54 and includes one or more light-emitting components, such as light-emitting diodes (LEDs), fluorescent lamps, incandescent bulbs, laser diodes, or plasma lights, that, when powered, (i) illuminate an area of the environment 54 proximate to or in vicinity of the respective lighting device 58, and (ii) deliver sufficient doses of visible light to inactivate pathogens (e.g., SARS-CoV-2, influenza A virus, MRSA bacteria, etc.) in the illuminated area, as will be described below. In some versions, a lighting device 58 has a downlight composed of an LED array, which may be contained within a housing. The housing may contain a heat sink, an LED module, optics, trim, and/or other components. In some versions, the downlight may be included as part of a movable structure to enable the lighting device to treat different portions of a target area (e.g., a surface, room, etc.). In one version, the lighting devices 58 can be uniformly constructed. In another version, the lighting devices 58 can vary in type, shape, and/or size. As an example, the lighting system 50 can employ various combinations of the different lighting devices described herein.

The bridge devices 62 are, at least in this example, located at the environment 54 and are communicatively connected (e.g., via wired and/or wireless connections) to one or more of the lighting devices 58. In the lighting system 50 illustrated in FIG. 6, four bridge devices 62 are utilized, with each bridge device 62 connected to three different lighting devices 58. In other examples, more or less bridge devices 62 can be connected to more or less lighting devices 58.

The server 66 may be any type of server, such as, for example, an application server, a database server, a file server, a web server, or other server). The server 66 may include one or more computers and/or may be part of a larger network of servers. The server 66 is communicatively connected (e.g., via wired and/or wireless connections) to the bridge devices 62. The server 66 can be located remotely (e.g., in the “cloud”) from the lighting devices 58 and the client devices 70 and may include one or more processors, controller modules (e.g., a central controller 76), or the like that are configured to facilitate various communications and commands among the client devices 70, the bridge devices 62, and the lighting devices 58. As such, the server 66 can generate and send commands or instructions to the lighting devices 58 to implement various sets of lighting settings corresponding to operation of the lighting devices 58. Each set of lighting settings may include various parameters or settings including, for example, spectral characteristics, operating modes (e.g., examination mode, disinfection mode, blended mode, nighttime mode, daytime mode, etc.), dim levels, output wattages, intensities, timeouts, and/or the like, whereby each set of lighting settings may also include a schedule or table specifying which settings should be used based on the time of day, day or week, natural light levels, occupancy, and/or other parameters. The server 66 can also receive and monitor data, such as operating status, light emission data (e.g., what and when light was emitted), hardware information, occupancy data, daylight levels, temperature, power consumption, and dosing data, from the lighting devices 58 via the bridge devices 62. In some cases, this data can be recorded and used to form or generate reports, e.g., a report indicative of the characteristics of the light emitted by one or more of the lighting devices 58. Such reports may, for example, be useful in evidencing that the environment 54 was, at or during various periods of time, delivering sufficient doses of visible light to inactivate pathogens in the illuminated area.

The network(s) 74 may be any type of wired, wireless, or wireless and wired network, such as, for example, a wide area network (WAN), a local area network (LAN), a personal area network (PAN), or other network. The network(s) 74 can facilitate any type of data communication via any standard or technology (e.g., GSM, CDMA, TDMA, WCDMA, LTE, EDGE, OFDM, GPRS, EV-DO, UWB, IEEE 802 including Ethernet, WiMAX, WiFi, Bluetooth®, and others).

The client device(s) 70 may be any type of electronic device, such as a smartphone, a desktop computer, a laptop, a tablet, a phablet, a smart watch, smart glasses, wearable electronics, a pager, a personal digital assistant, or any other electronic device, including computing devices configured for wireless radio frequency (RF) communication. The client device(s) 70 may support a graphical user interface (GUI), whereby a user of the client device(s) 70 may use the GUI to select various operations, change settings, view operation statuses and reports, make updates, configure email/text alert notifications, and/or perform other functions. The client device(s) 70 may transmit, via the network(s) 74, the server 66, and the bridge device(s) 62, any updated light settings to the lighting devices 58 for implementation and/or storage thereon. The client device(s) 70 may facilitate data communications via a gateway access point that may be connected to the bridge device(s) 62. In one implementation, the gateway access point may be a cellular access point that includes a gateway, an industrial Ethernet switch, and a cellular router integrated into a sealed enclosure. Further, the gateway access point may be secured using HTTPS with a self-signed certificate for access to web services, and may push/pull data between various websites, the one or more bridge devices 62, and the lighting devices 58.

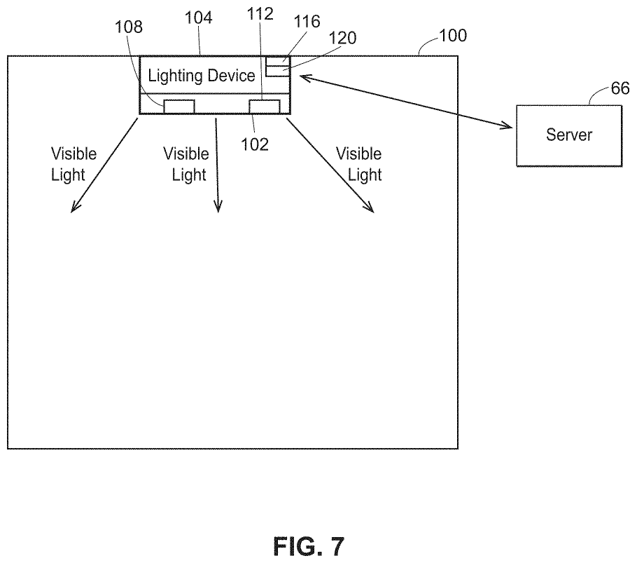

FIG. 7 illustrates a healthcare environment 100 that includes one of the lighting devices 58, taking the form of a lighting device 104 constructed in accordance with the present disclosure. The healthcare environment 100, which can, for example, be or include an examination room, an operating room, a bathroom, a hallway, a waiting room, a closet or other storage area, an emergency department, a clean room, or a portion thereof, is generally susceptible to the spread of dangerous pathogens, as discussed above.

Laboratory studies have shown that specially configured doses of narrow spectrum visible light (e.g., light having a wavelength between 400 nm and 420 nm, light having a wavelength of between 460 nm and 480 nm, light having a wavelength of between 530 nm and 580 nm, light having a wavelength of between 600 nm and 650 nm) can, when delivered in a sufficiently high amount (i.e., a sufficiently high amount of irradiating energy produced by applying a specified irradiation dose over a specified duration of time), effectively inactivate (i.e., “deactivate” or destroy) dangerous certain types of pathogens, e.g., MRSA bacteria. Moreover, the studies described in the present disclosure demonstrated that irradiation via 405 nm light is effective to inactivate lipid-enveloped viruses such as SARS-CoV-2 and influenza A virus (IAV). However, the doses required to inactivate dangerous pathogens tend to have a distracting or objectionable aesthetic impact in or upon the environment to which they are delivered. For example, these doses may provide an output of light that is undesirable when performing surgery in the healthcare environment 100. Thus, it has proven difficult to incorporate these doses into lighting devices that can simultaneously inactivate pathogens and illuminate an environment (e.g., the healthcare environment 100) in a non-objectionable manner. Instead, doses of narrow spectrum visible light are typically only delivered in when the environment is unoccupied, thereby severely limiting the inactivation potential of such lighting devices.

The lighting device 104 described herein is configured to deliver doses of narrow spectrum visible light at power levels sufficiently high enough to effectively inactivate dangerous pathogens in the healthcare environment 100 (or other environment), and, at the same time, provide visible light that sufficiently illuminates the environment 100 (or other environment) in a safe and unobjectionable manner. The lighting device 104 accomplishes both of these tasks without using a photosensitizer. The amount of 405 nm light required to inactivate bacterial organisms (e.g. S. aureus) has been integrated into normal overhead (i.e. white) lighting through the use of LED technology to safely provide both disinfection and illumination while the room is occupied. Organisms which are more difficult to inactivate, such as endospores, require levels of 405 nm light that can only be achieved through a single dedicated purpose device (i.e. disinfection or illumination). In these instances, the 405 nm disinfection in only applied to an unoccupied room due to the visually unappealing nature of this saturated color when applied in isolation from normal white light.

More specifically, the lighting device 104 provides or delivers (e.g., outputs, emits) at least 3,000 mW (or 3 W) of disinfecting light, which has a wavelength in the range of approximately 400 nm to approximately 420 nm (and, preferably, about 405 nm), a wavelength in the range of approximately 460 nm to 480 nm (e.g., a wavelength of about 470 nm), a wavelength in the range of 530 nm to 580 nm, a wavelength in the range of 600 nm to 650 nm, or combinations thereof, to the environment 100, as it will be appreciated that doses of light having a wavelength in these ranges but delivered at power levels lower than 3,000 mW are generally ineffective in inactivating dangerous pathogens. The lighting device 104 may, for example, provide or deliver 3,000 mW, 4,000 mW (or 4 W), 5,000 mW (or 5 W), 6,000 mW (or 6 W), 7,000 mW (or 7 W), 10,500 mW (or 10.5 W), or some other level of disinfecting light above 3,000 mW. Thus, for example, the light provided by the lighting device 104 may have a component of spectral energy measured in the 400 nm to 420 nm wavelength range that is greater than 10%, 15%, or 20%. In one example, the light may have a component of spectral energy measured in the 400 nm to 420 nm wavelength range that is greater than 16%. The lighting device 104 also provides or delivers levels of disinfecting light such that the air and any exposed surfaces within the environment 100 are subject to a desired, minimum power density while the lighting device 104 is used for inactivation, thereby ensuring that the environment 100 is adequately disinfected. This desired, minimum power density is the minimum power, measured in mW, received per unit area, measured in cm². This minimum power density within the applicable bandwidth(s) of visible light (e.g., 400-420 nm, 460-480 nm, 530-580 nm, 600-650 nm) may be referred to, as it is herein, as the minimum irradiance. The minimum irradiance (or “dose”) of the disinfecting light provided by the lighting device 104, which in this example is measured from any exposed surface or unshielded point (e.g., air) in the environment 100 that is 1.5 m from any point on any external-most luminous surface 102 of the lighting device 104 but may in other examples be measured from a different distance (e.g., 0.3 m) from any external-most luminous surface 102, nadir, any unshielded point in the environment 100, or some other point, is generally equal to a value between 0.01 mW/cm² and 10 mW/cm², or preferably, between 0.01 mW/cm² and 1.0 mW/cm², as irradiance values above 1.0 mW/cm² are likely to exceed the exposure limit prescribed by the IEC 62471 standard. More particularly, the minimum irradiance may be equal to a value between 0.035 mW/cm² and 0.6 mW/cm², in view of the considerable virucidal effects of these irradiances as demonstrated in the studies described herein. The minimum irradiance may, for example, be equal to 0.02 mW/cm², 0.035 mW/cm², 0.05 mW/cm², 0.076 mW/cm², 0.1 mW/cm², 0.15 mW/cm², 0.20 mW/cm², 0.25 mW/cm², 0.30 mW/cm², 0.35 mW/cm², 0.40 mW/cm², 0.45 mW/cm², 0.50 mW/cm², 0.55 mW/cm², 0.60 mW/cm², 0.65 mW/cm², 0.70 mW/cm², 0.75 mW/cm², 0.80 mW/cm², 0.85 mW/cm², 0.90 mW/cm², 0.95 mW/cm², 1.00 mW/cm², or some other value in the above-specified ranges. When the minimum irradiance of the disinfecting light provided by the lighting device 104 is measured or determined over time (the period of time over which the lighting device 104 is used for inactivation), the exposed surfaces or unshielded points in the environment 100 may be subject to a total disinfecting energy that is equal to at least 0.06 J/cm², which laboratory studies have shown is sufficient for inactivating certain dangerous pathogens in the environment 100. Additionally, or alternatively, the total disinfecting energy may be an energy value achieved by providing 400-420 nm light with a peak wavelength of 405 nm at a dose of 0.035 mW/cm², 0.076 mW/cm², 0.15 mW/cm², or 0.6 mW/cm² over a duration of approximately one, two, four, eight, 12, 24, or any other number of hours, as has shown to be effective to inactivate SARS-CoV-2 and IAV.

At the same time, the lighting device 104 provides an output of visible light that is aesthetically pleasing, or at least unobjectionable, to humans (e.g., patients, personnel) in and around the environment 100. In some applications, the lighting device 104 may provide an output of visible light that is perceived by humans in and around the environment 100 as white light, with properties that studies have shown to be aesthetically pleasing, or at least unobjectionable, to humans, and has a disinfection component including disinfecting light (i.e., the narrow spectrum visible light discussed above). While the exact properties of the white light may vary depending on the given application, the properties generally include one or more of the following: (1) a desirable color rendering index, e.g., a color rendering index of greater than 70, greater than 80, or greater than 90; (2) a desirable correlated color temperature, e.g., a color temperature of between approximately 1500 degrees Kelvin and 7000 degrees Kelvin, more particularly between approximately 1800 degrees and 5000 degrees Kelvin, between approximately 2100 degrees and 6000 degrees Kelvin, between approximately 2700 degrees and 5000 degrees Kelvin, or some other temperature or range of temperatures within these ranges or partially or totally outside of these ranges; or (3) a desirable chromaticity. In other applications, the lighting device 104 may provide an output of visible light that is perceived by humans in and around the environment 100 as unobjectionable non-white light, with properties that studies have shown to be aesthetically pleasing, or at least unobjectionable, to humans, and has a disinfection component including disinfecting light. As an example, the output of visible light may be non-white, but also non-violet, light. It will be appreciated that the output of visible light may be entirely formed of disinfecting light that is mixed together in a manner that yields unobjectionable non-white light or only partially formed of disinfecting light that is mixed with non-disinfecting light in a manner that yields unobjectionable non-white light. As with white light, the exact properties of the unobjectionable non-white light may vary depending on the given application, but the properties generally include one or more of the following: (1) a desirable color rendering index, e.g., a color rendering index of greater than 70, greater than 80, or greater than 90; (2) a desirable color temperature, e.g., a color temperature of between approximately 1500 degrees Kelvin and 7000 degrees Kelvin, more particularly between approximately 1800 degrees and 5000 degrees Kelvin, between approximately 2100 degrees and 6000 degrees Kelvin, between approximately 2700 degrees and 5000 degrees Kelvin, or some other temperature or range of temperatures within these ranges or partially or totally outside of these ranges; or (3) a desirable chromaticity.

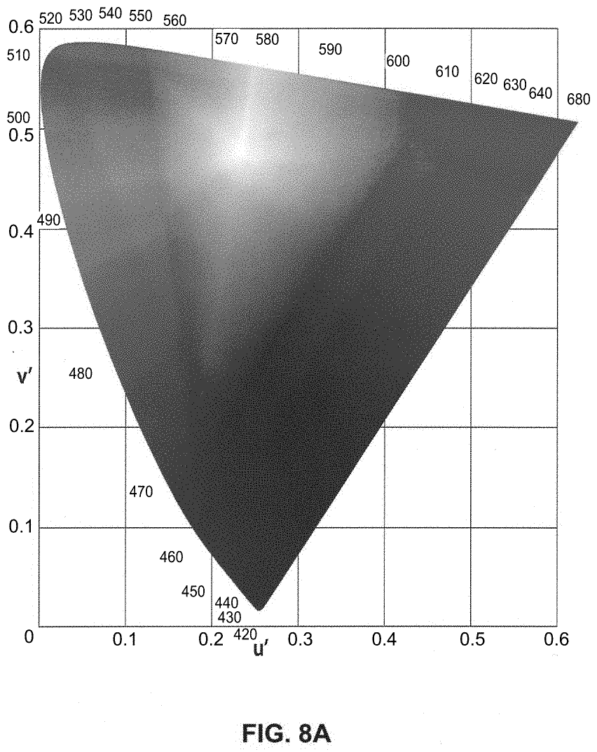

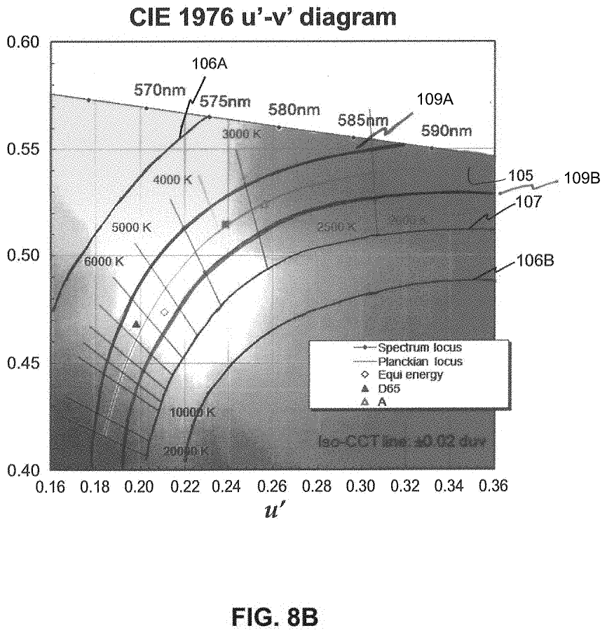

Chromaticity can be described relative to any number of different chromaticity diagrams, such as, for example, the 1931 CIE Chromaticity Diagram, the 1960 CIE Chromaticity Diagram, or the 1976 CIE Chromaticity Diagram shown in FIG. 8A. The aesthetically pleasing light output by the lighting device 104 can thus be described as having properties relative to or based on these chromaticity diagrams. As illustrated in, for example, FIG. 8B, the lighting device 104 may output white light having u′, v′ coordinates on the 1976 CIE Chromaticity Diagram (FIG. 8A) that lie on any number of different curves relative to a planckian locus 105 defined by the ANSI C78.377-2015 color standard. The ANSI C78.377-2015 color standard generally describes the range of color mixing that creates pleasing, or visually appealing, white light. This range is generally defined by the planckian locus 105, which is also known as a blackbody curve, with some deviation, measured in Duv, above or below the planckian locus 105. The different curves on which the u′, v′ coordinates of the white light output can lie deviate from the planckian locus 106 by different Duv values, depending upon the given application. The white light may, for example, lie on a curve 106A that is 0.035 Duv above the planckian locus 105, on a curve 106B that is 0.035 Duv below (−0.035 Duv) the planckian locus 105, on a curve 107 that is 0.02 Duv below (−0.02 Duv) the planckian locus 105, on a curve that is 0.02 Duv above the planckian locus, or some other curve between 0.035 Duv above and 0.035 Duv below the planckian locus 105. As also illustrated in FIG. 8B, the lighting device 104 may, for example, output non-white light having u′, v′ coordinates on the 1976 CIE Chromaticity Diagram that lie outside of an area that is bounded (i) vertically between the curve 106A and the curve 106B, a curve 109A that is 0.007 Duv above the planckian locus 105 and a curve 109B that is 0.007 Duv below (−0.007 Duv) the planckian locus 105, or other curves, and (ii) horizontally between a color temperature isoline of between approximately 1500 K and 7000 K.

The lighting device 104 is, in some cases, fully enclosed, which promotes cleanliness, by, for example, preventing pathogens from nesting on or within internal components of the lighting device 104, which would otherwise be hard to reach with the specially configured narrow spectrum visible light. In other words, in these cases, no surface internal to the lighting device 104 is exposed to the environment 100 surrounding the lighting device 104, such that dangerous pathogens cannot reside on surfaces hidden from the narrow spectrum visible light.

As will be described herein, the lighting device 104 includes one or more light-emitting elements, e.g., light-emitting diodes (LEDs), configured to emit light as desired. The lighting device 104 optionally includes means for directing the emitted light. The means for directing the emitted light may, for example, include one or more reflectors, one or more lenses, one or more diffusers, and/or one or more other components. In some examples, e.g., when LEDs are employed in the lighting device, the lighting device 104 can include a means for maintaining a junction temperature of the LEDs below a maximum operating temperature of the LEDs. The means for maintaining a junction temperature may, for example, include one or more heat sinks, one or more metallic bands, spreading heat to printed circuit boards coupled to the LEDs, a constant-current driver topology, a thermal feedback system to one or more drivers (that power the LEDs) via NTC thermistor, or other means that reduce LED drive current at sensed elevated temperatures. Moreover, the lighting device 104 optionally includes means for creating air convection proximate to the lighting device 104 so as to facilitate circulation of disinfected air away from the lighting device 104 and air that has not been disinfected toward the lighting device 104. The means for creating air convection may, for example, include one or more fans (part of or separate from the lighting device 104), one or more heat sinks, one or more channels formed in the lighting device 104, or other means. The lighting device 104 can further include an occupancy sensor 108, a daylight sensor 112, one or more communication modules 116, and one or more control components 120, e.g., a local controller. The lighting device 104 can optionally include one or more additional sensors, e.g., two occupancy sensors 108, a sensor that measures the light output by the device 104, etc.

In this version, the occupancy sensor 108 is an infrared (IR) motion sensor that detects motion within a pre-determined range of or distance from (e.g., 50 feet) the lighting device 104, so as to identify (or help identify) whether the environment 100 is occupied or is vacant (i.e., not occupied) and has been occupied or vacant for a period of time (e.g., a predetermined period of time, such as 15 minutes, 30 minutes, etc.). The occupancy sensor 108 may continuously monitor the environment 100 to determine whether the environment 100 is occupied. In other versions, the occupancy sensor 108 can be a different type of sensor, e.g., an ultrasonic sensor, a microwave sensor, a CO₂ sensor, a thermal imaging sensor, that utilizes a different occupancy detection technique or technology to identify (or help identify) whether the environment 100 is or is not occupied and has or has not been occupied for a period of time. In some versions, multiple occupancy sensors 108 that detect occupancy using different detection techniques or technologies can be employed to provide for a more robust detection. As an example, the lighting device 104 can include one infrared motion sensor and one CO2 sensor, which utilize different techniques or technologies to detect occupancy. The daylight sensor 112, meanwhile, is configured to detect natural light within a pre-determined range of or distance from (e.g., 50 feet) the lighting device 104, so as to identify whether it is daytime or nighttime (and thus, whether the environment 100 is or is not occupied).

The lighting device 104 can, responsive to occupancy data obtained by the occupancy sensor 108 and/or natural light data obtained by the daylight sensor 112, be controlled by the local controller 120 (or other control components) to emit visible light of or having various characteristics. The lighting device 104 can, for example, responsive to data indicating that the environment 100 is vacant (i.e., not occupied), be controlled so as to output visible light consisting only of the specially configured narrow spectrum visible light. In some cases, the narrow spectrum visible light is only output after the lighting device 104 determines that the environment 100 has been vacant for a pre-determined period of time (e.g., 30 minutes), thereby providing a fail-safe that ensures that the environment 100 is indeed vacant. The lighting device 104 can, via the communication module(s) 116, be communicatively connected to and controlled by the remotely located server 66 (as well as remotely located client devices 70) and/or be communicatively connected to other lighting devices 58. As such, the lighting device 104 may transmit data, such as operating status (e.g., the operating mode), light emission data, hardware information, occupancy data, daylight levels, output wattages, temperature, power consumption, to the server 66 and/or other lighting devices 58, and may receive, from the server 66, other lighting devices 58, and/or the client devices 70, operational instructions (e.g., turn on, turn off, provide light of a different spectral characteristic, switch between operating modes) and/or other data (e.g., operational data from or about the other lighting devices 58).

It will be appreciated that the lighting device 104 can be manually controlled (e.g., by a user of the lighting device 104) and/or automatically controlled responsive to other settings, parameters, or data in place of or in addition to the data obtained by the occupancy sensor 108 and/or the daylight sensor 112. The lighting device 104 may, for example, be partially or entirely controlled by the local controller 120 (or other control components) responsive to an operating mode, a dim level, a schedule or a table, or other parameter(s) or setting(s) received by the local controller 120 (or other control component(s)).

In some versions, such as the one illustrated in FIG. 7, the lighting device 104 can include a dosing or inactivation feedback system 124 that monitors and records the amount and frequency of dosing and amount of inactivating energy delivered by the lighting device 104. The dosing feedback system 124 is, in this version, implemented by the local controller 120, though the dosing feedback system 124 can be implemented using other components (e.g., a suitable processor and memory) in the lighting device 104 or can be implemented via the server 66. In any event, the dosing feedback system 124 achieves the aforementioned aims by monitoring and recording the various parameters or settings of and associated with the lighting device 104 over a period of time. More specifically, the dosing feedback system 124 monitors and records the spectral characteristics, the output wattages, wavelengths, and/or intensities of the light (or components thereof) emitted by the lighting device 104, the minimum irradiance of the disinfecting narrow spectrum visible light provided by the lighting device 104, occupancy data obtained by the occupancy sensor 108, the amount of time the lighting device 104 has spent in various operating modes (e.g., examination mode), dim levels, and the like. As an example, the dosing feedback system 124 monitors and records when the lighting device 104 emits visible light that includes or solely consists of disinfecting narrow spectrum visible light (e.g., light having a wavelength between 400 nm and 420 nm, light having a wavelength between 460 nm and 480 nm, light having a wavelength of between 530 nm and 580 nm, light having a wavelength of between 600 nm and 650 nm, or combinations thereof), as well as the levels and density (and more particularly the minimum irradiance) of disinfecting narrow spectrum visible light delivered during those times. Based on the parameters or settings of the lighting device 104, the dosing feedback system 124 (and/or an operator of the lighting device 104) can determine the quantity and frequency of inactivation dosing delivered by the lighting device 104. Alternatively or additionally, the dosing feedback system 124 can provide the recorded data to the server 66 (via the communication module(s) 116), which can in turn determine the quantity and frequency of inactivation dosing delivered by the lighting device 104. In some cases, the dosing feedback system 124 and/or the server 66 can generate periodic reports including the obtained data and/or determinations with respect to inactivation dosing. When the dosing feedback system 124 generates these reports, the reports can be transmitted to the server 66 or any other component via the communication module(s) 116. In any case, the dosing feedback system 124 allows a hospital or other environment 100 that implements the lighting device 104 to quantitatively determine (and verify) that sufficient levels of inactivating energy were delivered over various periods of time or at certain points in time (e.g., during a particular operation). This can, for example, be extremely beneficial in the event that the hospital or other environment 100 is sued by a patient alleging that she/he acquired a HAI while at the hospital or other environment 100.

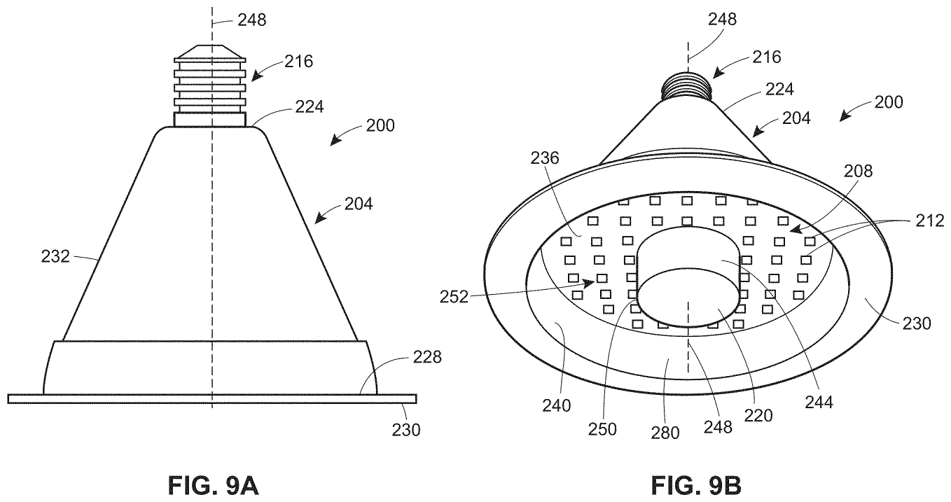

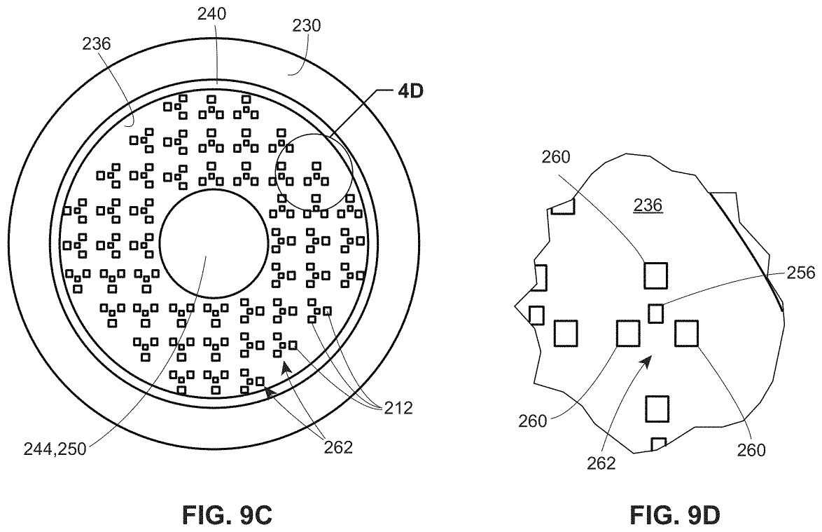

As illustrated in FIGS. 9A-9C, the lighting device 104 can take the form of a light bulb or fixture 200. The light fixture 200 includes an enclosed housing 204, an array 208 of light-emitting elements 212 coupled to (e.g., installed or mounted on) a portion of the housing 204, a base 216 coupled to (e.g., integrally formed with) the housing 204, and an occupancy sensor 220 coupled to (e.g., disposed or arranged on) a portion of the housing 204. The occupancy sensor 220 is optimally positioned to detect motion within a pre-determined range of or distance from (e.g., 50 feet) the light 200 within the environment 100. The light fixture 200 can emit light responsive to detection data obtained by the occupancy sensor 220, as will be discussed in greater detail below.

The housing 204 is, as noted above, enclosed, thereby preventing moisture ingress into the light fixture 200 and/or contamination of the internal components of the light fixture 200. More specifically, no surface internal to the housing 204 is exposed to the environment 100, such that dangerous pathogens cannot reside on surfaces hidden from the inactivating light emitted by the light device 200. The housing 204 illustrated in FIGS. 9A-9C is made of or manufactured from aluminum or stainless steel and has a first end 224, a second end 228, an outwardly extending annular flange 230 formed at the second end 228, and an outer circumferential wall 232 extending between the first and second ends 224, 228. The outer circumferential wall 232 has a substantially conical shape, with the diameter of the circumferential wall 232 increasing in a direction from the first end 224 to the second end 228, such that the diameter of the wall 232 is larger at the second end 228 than at the first end 224.

The housing 204 also includes a circular support surface 236 and an inner circumferential wall 240 surrounding the support surface 236. The support surface 236, which at least in FIG. 9B faces downward, is arranged to receive a portion or all of the array 208 of the light-emitting elements 212. The inner circumferential wall 240, like the outer circumferential wall 232, has a substantially conical shape. The inner circumferential wall 240 is spaced radially inward of the outer circumferential wall 232 and extends between the flange 230 of the housing 204 and the support surface 236.

The housing 204 also includes a support element, which in this version takes the form of a cylindrical post 244, disposed along a center axis 248 of the light 200. The cylindrical post 244 extends outward (downward when viewed in FIG. 9B) from the support surface 236 and terminates at an end 250 positioned axially inward of the second end 228 (i.e., axially located between the first and second ends 224, 228). A cavity 252 is formed or defined proximate to the second end 228 and between the flange 230, the inner circumferential wall 240, and the cylindrical post 244.

The array 208 of light-emitting elements 212 is generally arranged on or within the enclosed housing 204. The array 208 of light-emitting elements 212 is, in this version, arranged on an outer portion of the enclosed housing 204 exposed to the environment 100. More specifically, the light-emitting elements 212 are arranged in the cavity 252, on the support surface 236 and surrounding the post 244, as illustrated in FIGS. 9B and 9C. The light-emitting elements 212 can be secured in any known manner (e.g., using fasteners, adhesives, etc.). Any number of light-emitting elements 212 can be utilized, depending on the given application (e.g., depending upon the healthcare environment 100. As an example, more light-emitting elements 212 may be utilized for larger environments 100 and/or for environments 100 particularly susceptible to high levels of dangerous pathogens.

The light-emitting elements 212 include one or more first light-emitting elements 256 and one or more second light-emitting elements 260 arranged in any number of different patterns. The light-emitting elements 212 illustrated in FIGS. 9C and 9D include a plurality of clusters 262 each having one first light-emitting element 256 surrounded by three second light-emitting elements 260. However, in other examples, the light-emitting elements 212 can be arranged differently, for example, with one or more of the clusters 262 having a different arrangement of the light-emitting elements 256 and the second light-emitting elements 260. The light-emitting elements 256 in this version take the form of light-emitting diodes (LEDs) and are configured to together (i.e., combine to) emit at least 3,000 mW of specially configured visible light, in this case light having a wavelength in a range of between approximately 400 nm and approximately 420 nm (e.g., with a peak wavelength of 405 nm). In some cases, the light-emitting elements 256 can be configured to together emit at least 5,000 mW of specially configured visible light, while in other cases, the light-emitting elements can be configured to together emit at least 10,500 mW of specially configured visible light. The light-emitting elements 260 also take the form of LEDs, at least in this version, but are configured to emit visible light that complements the visible light emitted by the light-emitted elements 256. Generally speaking, the light emitted by the light-emitting elements 260 has a wavelength greater than the wavelength of the light emitted by the light-emitting elements 256. In many cases, the light emitted by some, if not all, of the light-emitting elements 260 will have a wavelength greater than 500 nm. As an example, the light-emitting elements 260 may emit red, green, and blue light, which combine to yield or form white visible light. The total light emitted by the light-emitting elements 256 has, in many cases, a greater luminous flux than the total light emitted by the light-emitting elements 260, though this need not be the case.

In any event, the light-emitting elements 256 and 260 are configured such that the total or combined light emitted by the array 208 is white, a shade of white, or a different color that is aesthetically non-objectionable in the healthcare environment 100. Generally speaking, the total or combined light will have a color rendering index of above 70, and, more preferably, above 80 or above 90, and will have a color temperature in a range of between 1500 degrees and 7000 degrees Kelvin, preferably in a range of between 2100 degrees and 6000 degrees Kelvin, and, more preferably, in a range of between 2700 degrees and 5000 degrees Kelvin.

The base 216 is coupled proximate to, and protrudes outward from, the first end 224 of the housing 204. The base 216 in this version is a threaded base that is integrally formed with the housing 204 and is adapted to be screwed into a matching socket (not shown) provided in a receiving structure in the healthcare environment 100. The matching socket can be provided in a wall, a ceiling, a floor, a housing, or some other structure, depending upon the healthcare environment 100. In any event, as is known in the art, the threaded base 216 can include one or more electrical contacts adapted to be electrically connected to corresponding electrical contacts of the socket when the base 216 is coupled to the socket, thereby powering the light fixture 200.

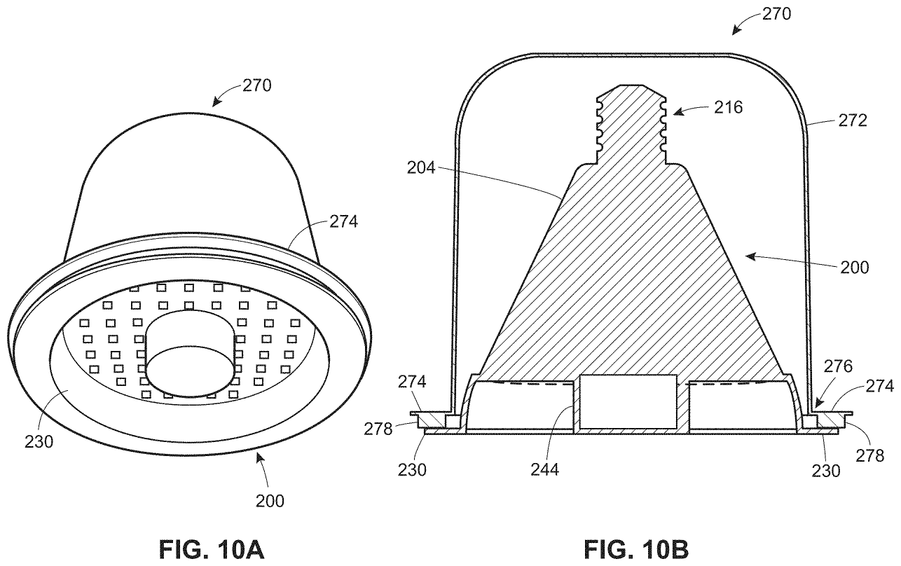

It is generally desired that the base 216 be screwed into the matching socket such that at least a portion of the housing 204 is recessed into the discrete structure, thereby sealing that portion of the housing 204 from the external environment. FIGS. 10A and 10B illustrate an example of this, wherein the light fixture 200 is sealingly disposed in a receiving structure 270 provided (e.g., formed) in a ceiling, housing, or other structure in the environment 100. The receiving structure 270 has a substantially cylindrical base 272 and an outwardly extending flange 274 formed at an end 276 of the base 272. A seal (e.g., a gasket) 278 is disposed on the outwardly extending flange 274 of the receiving structure 270. When the base 216 of the light fixture 200 is screwed into a matching socket (not shown) provided in the receiving structure 270, the housing 204 of the light fixture 200 is substantially entirely disposed or recessed within the base 272 of the receiving structure 270, and the flange 230 of the light 200 sealingly engages the seal 278 disposed on the flange 274 of the receiving structure 270. In this way, the housing 204 is substantially sealed off from the outside environment 100.

With reference back to FIGS. 9A and 9B, the occupancy sensor 220, which can take the form of a passive infrared motion sensor, a microwave motion sensor, an ultrasonic motion sensor, or another type of occupancy sensor, is arranged or disposed on a downward facing portion of the housing 204. The occupancy sensor 220 in this version is disposed on the end 250 of the cylindrical post 244, which allows the occupancy sensor 220 to detect motion within a pre-determined range of or distance from (e.g., 50 feet) the light device 200 within the environment 100. In some cases, the occupancy sensor 220 can detect any motion within the environment 100 (e.g., when the environment 100 only includes one light fixture 200). As briefly discussed above, the light 200 can emit light responsive to detection data obtained by the occupancy sensor 220. More specifically, the light fixture 200 can adjust the outputted light in response to detection data obtained by the occupancy sensor 220. When, for example, the occupancy sensor 220 does not detect any motion within the pre-determined range or distance, the light device 200 device can shut off or emit less light from the second light-emitting elements 260, as the healthcare environment 100 is not occupied (and, therefore, the color of the emitted light may not matter). In other words, the light 200 can emit light only from the first light-emitting elements 256, thereby inactivating dangerous pathogens while using less power. Conversely, when the occupancy sensor 220 detects motion within the pre-determined range or distance, the light fixture 200 can emit light from both the first and second light-emitting elements 256, 260, thereby ensuring that the aesthetically unobjectionable light (e.g., white light) is provided to the occupied healthcare environment 100 and, at the same time, the light fixture 200 continues to inactivate dangerous pathogens, even while the environment 100 is occupied.

With reference still to FIGS. 9A and 9B, the light fixture or bulb 200 also includes an annular refractor 280. The refractor 280 in this version is a nano-replicated refractor film mounted to the inner circumferential wall 240 of the housing 204. The refractor 280 can be secured there via any known manner (e.g., using a plurality of fasteners, using adhesives, etc.). So disposed, the refractors 280 surrounds or circumscribes the first and second light-emitting elements 256, 260, such that the refractor 280 helps to focus and evenly distribute light emitted from the light 200 to the environment 100. If desired, the refractor 280 can be arranged differently or other types of refractors can instead be utilized so as to yield different controlled light distributions.

Although not depicted herein, it will be understood that one or more drivers (e.g., LED drivers), one or more other sensors (e.g., a daylight sensor), one or more lenses, one or more reflectors, one or more boards (e.g., a printed circuit board, a user interface board), wiring, various control components (e.g., a local controller communicatively connected to the server 66), one or more communication modules (e.g., one or more antennae, one or more receivers, one or more transmitters), and/or other electrical components can be arranged or disposed within or proximate to the enclosed housing 204. The communication modules can include one or more wireless communication modules and/or one or more wired communication modules. The one or more communication modules can thus facilitate wireless and/or wired communication, using any known communication protocol(s), between components of the light bulb or fixture 200 and the local controller, the server 66, and/or other control system components. More specifically, the one or more communication modules can facilitate the transfer of various data, such as occupancy or motion data, operational instructions (e.g., turn on, turn off, dim, etc.), etc., between the components of the bulb or fixture 200 and the local controller, the server 66, other lighting devices 58, and/or other control system components. For example, data indicative of when light is emitted from the light-emitting elements 256, 260 can be monitored and transmitted to the server 66 via such communication modules. As another example, data indicative of how much light is emitted from the light-emitting elements 256, 260 over a pre-determined period of time (e.g., during a specific surgical procedure) can be monitored and transmitted to the server 66 via such communication modules.

In other versions, the light bulb or fixture 200 can be constructed differently. Specifically, the housing 204 can have a different size, shape, and/or be made of one or more materials other than or in addition to aluminum or stainless steel. For example, the housing 204 can have a rectangular, square, triangular, irregular, or other suitable shape. In one version, the housing 204 may not include the post 244 and/or the post 244 may take on a different shape and/or size than the cylindrical post 244 illustrated in FIGS. 9A and 9B.

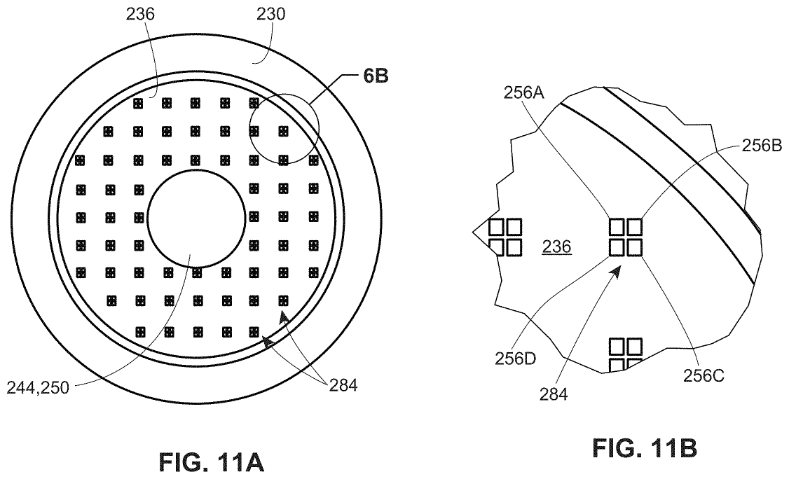

Moreover, the array 208 of light-emitting elements 212 can vary. In some versions, the array 208 (or portions thereof) can be arranged within or on a different portion of the housing 204. In some versions, the array 208 of light-emitting elements 212 may only include the first light-emitting elements 256, which, as noted above, are configured to emit specially configured spectrum visible light at a sufficiently high power level. In these versions, one or more of the light-emitting elements 256 can be covered or coated with phosphors, substrates infused with phosphors, and/or one or more other materials and/or media so as to yield light having a higher wavelength than the specially configured narrow spectrum visible light, such that the total or combined light emitted by the array 208 is white, a shade of white, or a different color that is aesthetically non-objectionable in the healthcare environment 100. FIGS. 11A and 11 B depict one such version, wherein the light-emitting elements 212 include a plurality of clusters 284 of four light-emitting elements 256, with three of the light-emitting elements 256A, 256B, and 256C being covered or coated with phosphors, and one of the light-emitting elements 256D being uncovered (i.e., not coated with a phosphor). In the illustrated version, the three light-emitting elements 256A, 256B, and 256C are covered or coated with blue, red, and green phosphors, respectively, such that the total or combined light emitted by each cluster 284 (and, thus, the array 208) is white, a shade of white, or a different color (i.e., non-white) that is aesthetically non-objectionable in the healthcare environment 100. It will be appreciated that in other versions, more or less of the light-emitting elements 256 can be covered with phosphors, the light-emitting elements 256 can be covered with different colored phosphors, and/or the light-emitting elements 256 can be arranged differently relative to one another (i.e., the clusters 284 can vary). In yet other versions, the array 208 can include additional light-emitting elements, e.g., LEDs configured to emit specially configured visible light at a sufficiently high power level, configured to be turned on only when no motion is detected in the environment 100 (for even greater room dosage). Finally, it will be appreciated that the first and/or second light-emitting elements 256, 260 can, instead of being LEDs, take the form of fluorescent, incandescent, plasma, or other light-elements.

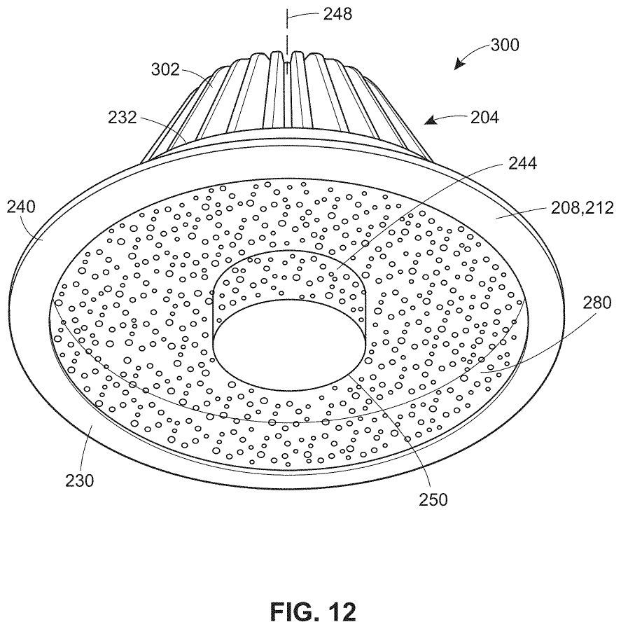

FIG. 12 illustrates another version of the lighting device 104. As illustrated in FIG. 12, the lighting device 104 can take the form of a light bulb or fixture 300. The light fixture 300 is substantially similar to the light fixture 200, with common reference numerals used to refer to common components. However, unlike the light 200, the light 300 includes a heat sink 302 formed on an exterior surface of the light 300 and configured to dissipate heat generated by the light fixture 300, and, more particularly, the light-emitting elements 212. In some cases, the heat sink 302 can be coupled (e.g., mounted, attached) to and around a portion of the outer circumferential wall 232, while in other cases the heat sink 302 can be integrally formed with the housing 204 (in which case the heat sink 302 may take the place of some or all of the wall 232).

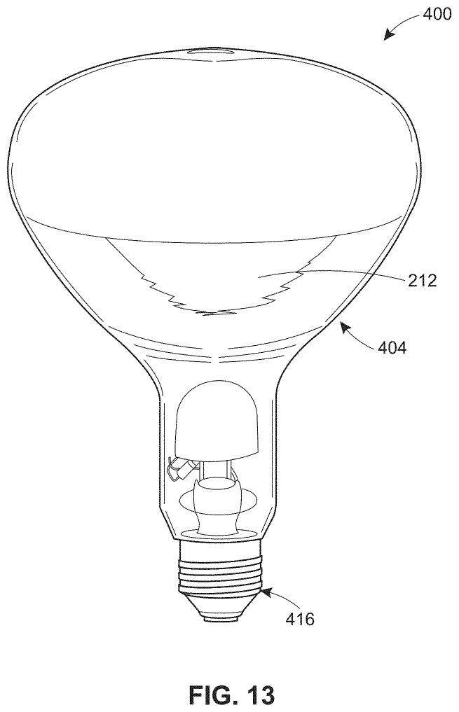

FIG. 13 illustrates yet another version of the lighting device 104. As illustrated in FIG. 12, the lighting device 104 can take the form of a light bulb or fixture 400. The light 400 includes an enclosed housing 404 that is different from the housing 204 of the lights 200, 300. The enclosed housing 404 is, in this version, is made of or manufactured from glass or plastic and is shaped like a housing of a conventional incandescent light bulb. The light 400 also includes a base 416, which is similar to the base 216 described above. However, unlike a conventional incandescent light bulb, the light 400 also includes the light-emitting elements 212, which are arranged within the enclosed housing 404 and, as discussed above, are configured to provide specially configured narrow spectrum visible light at power levels sufficiently high enough to effectively inactivate dangerous pathogens (e.g., bacteria and/or lipid-enveloped viruses), all while providing an output of quality light that is unobjectionable.

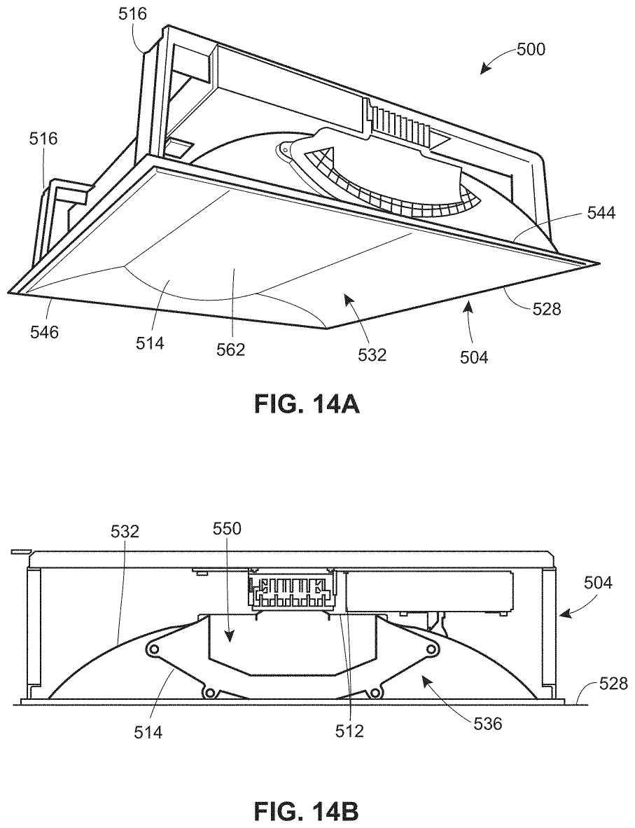

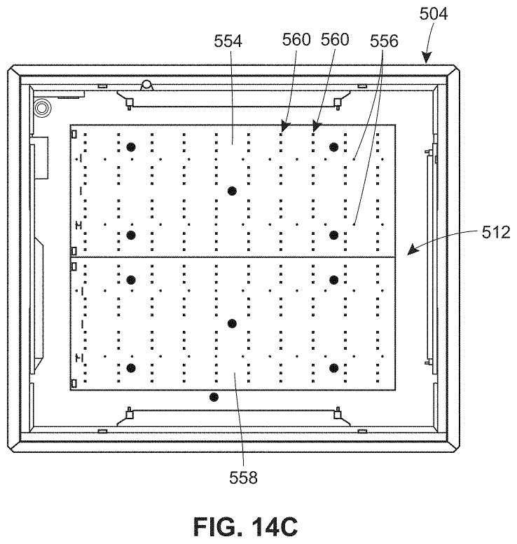

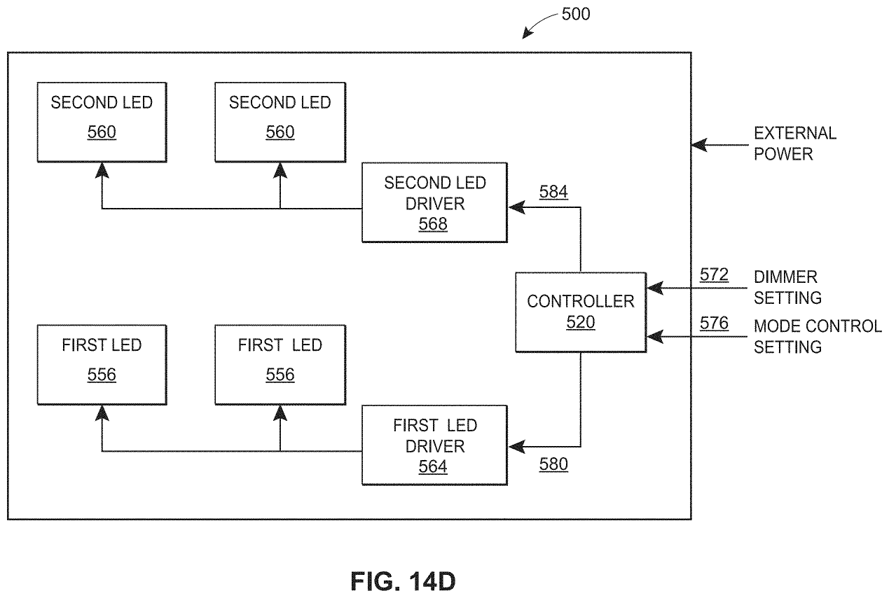

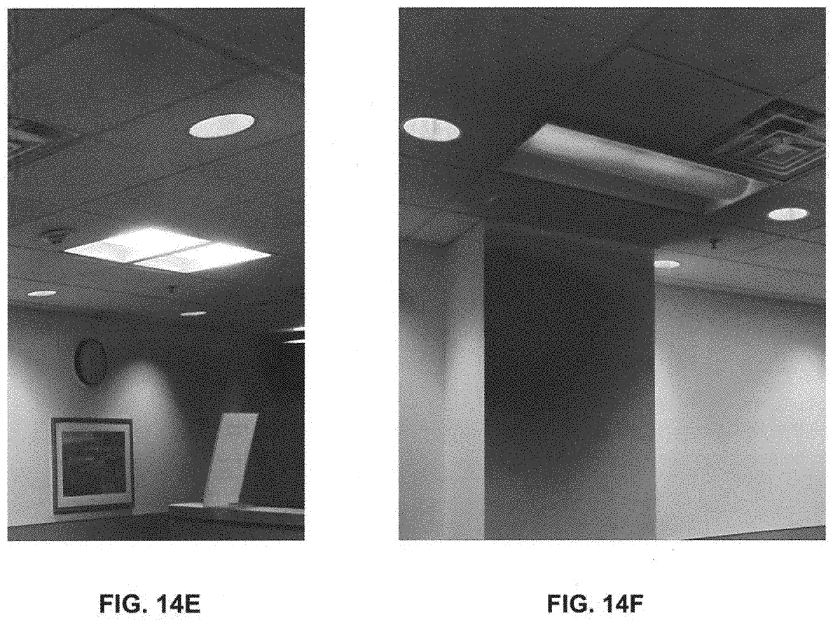

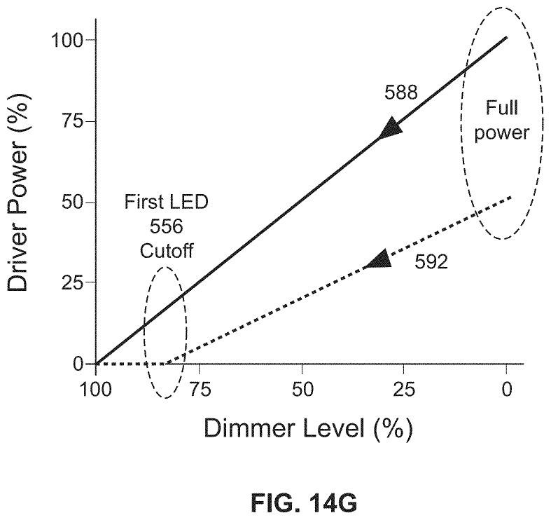

FIGS. 14A-14D illustrate yet another version of the lighting device 104, in the form of a light fixture 500. The light fixture 500 includes a housing or chassis 504, a plurality of light-emitting elements 512 coupled to (e.g., installed or mounted on) a portion of the housing 504, a lens 514 configured to diffuse light emitted by the light-emitting elements 512 in an efficient manner, a pair of support arms 516 coupled to (e.g., integrally formed with) the housing 504, and a control device in the form of a local controller 520 that is identical to the controller 120 described above. It will be appreciated that the light fixture 500 also includes an occupancy sensor, a daylight sensor, a communication module, and a dosing feedback system; these components are, however, identical to the motion sensor 108, the daylight sensor 112, the communication module 116, and the dosing feedback system 124, respectively, described above, so are, for the sake of brevity, not illustrated in FIGS. 14A-14C and are not described in any further detail below. The light fixture 500 may also include any of the means for maintaining junction temperature discussed above in connection with the lighting device 104.

The housing 504 in this version is made of or manufactured from steel (e.g., 18-gauge welded cold-rolled steel) and has a substantially rectangular flange 528 that surrounds a curved, interior support surface 532, which at least in FIG. 14B faces downward. The rectangular flange 528 and the curved, interior support surface 532 together define a cavity 536 sized to receive the lens 514, which in this example is a Frost DR Acrylic lens manufactured by Kenall Manufacturing. The support arms 516 are coupled to an exterior portion of the housing 504 proximate to the flange 528, with one support arm 516 coupled at or proximate to a first end 544 of the housing 504 and the other support arm 516 coupled at or proximate to a second end 546 of the housing 504 opposite the first end 536. The support arms 516 are thus arranged to facilitate installation of the light fixture 500, e.g., within a ceiling of the environment 100.

The light-emitting elements 512 are generally arranged on or within the housing 504. The light-emitting elements 512 are, in this version, arranged in a sealed or closed light-mixing chamber 550 defined by the housing 504 and the lens 540. The light-emitting elements 512 can be secured therein any known manner (e.g., using fasteners, adhesives, etc.). The light-emitting elements 512 in this version include a plurality of first light-emitting elements in the form of a plurality of first LEDs 556 and a plurality of second light-emitting elements in the form of a plurality of second LEDs 560. The light-emitting elements 512 can be arranged on first and second LED modules 554, 558 in the manner illustrated in FIG. 14C, with the second LEDs 560 clustered together in various rows and columns, and the first LEDs 556 arranged between these rows and columns, or can be arranged in a different manner. In one example, ninety-six (96) first LEDs 556 and five-hundred seventy-six (576) second LEDs 560 are used, for a ratio of first LEDs 556 to second LEDs 560 equal to 1:6. In other examples, more or less first and second LEDs 556, 560 can be employed, with different ratios of first LEDs 556 to second LEDs 560. As an example, the ratio of first LEDs 556 to second LEDs 560 may be equal to 1:3, 1:2, 1:1, or some other ratio, depending upon the power capabilities of the first and second LEDs 556, 560.

The first LEDs 556 are, like the light-emitting elements 256, configured to provide (e.g., emit) specially configured visible light, in this case light having a wavelength in a range of between approximately 400 nm and approximately 420 nm (e.g., 405 nm light), with the combination or sum of the first LEDs 556 configured to provide or deliver (e.g., emit) sufficiently high levels of the specially configured visible light so as to inactivate pathogens surrounding the light fixture 500. As discussed above, the first LEDs 556 may together (i.e., when summed) emit at least 3,000 mW of the specially configured visible light, e.g., 3,000 mW, 4,000 mW, 5,000 mW, or some other level of visible light above 3,000 mW. The minimum irradiance of the specially configured visible light emitted or otherwise provided by all of the LEDs 556, which, at least in this example, is measured from any exposed surface or unshielded point in the environment 100 that is 1.5 m from any point on any external-most luminous surface 562 of the lighting device 504, may be equal to a value between 0.01 mW/cm² and 10 mW/cm², or preferably, between 0.01 mW/cm² and 1.0 mW/cm², as irradiance values above 1.0 mW/cm² are likely to exceed the exposure limit prescribed by the IEC 62471 standard. More particularly, the minimum irradiance may be equal to a value between 0.035 mW/cm² and 0.6 mW/cm², in view of the considerable virucidal effects of these irradiances as demonstrated in the studies described herein. The minimum irradiance may, for example, be equal to 0.01 mW/cm², 0.02 mW/cm², 0.035 mW/cm², 0.05 mW/cm², 0.076 mW/cm², 0.1 mW/cm², 0.15 mW/cm², 0.20 mW/cm², 0.25 mW/cm², 0.30 mW/cm², 0.35 mW/cm², 0.40 mW/cm², 0.45 mW/cm², 0.50 mW/cm², 0.55 mW/cm², 0.60 mW/cm², 0.65 mW/cm², 0.70 mW/cm², 0.75 mW/cm², 0.80 mW/cm², 0.85 mW/cm², 0.90 mW/cm², 0.95 mW/cm², 1.00 mW/cm², or some other value in the above-specified ranges. In other examples, the minimum irradiance of the specially configured visible light may be measured from a different distance from any external-most luminous surface 562, nadir, or any other unshielded or exposed surface in the environment 100. The second LEDs 560 are, like the light-emitting elements 260, configured to emit visible light, but the second LEDs 560 emit light having a wavelength that is greater than the wavelength of the light emitted by the one or more first LEDs 556. The light emitted by the second LEDs 560 will generally have a wavelength that is greater than 500 nm, though this need not be the case.

In any event, the light emitted by the second LEDs 560 complements the visible light emitted by the one or more first LEDs 556, such that the combined or blended light output formed in the mixing chamber 550 is a white light having the properties discussed above (e.g., white light having a CRI of above 80, a color temperature in a range of between 2100 degrees and 6000 degrees, and/or (u′,v′) coordinates on the 1976 CIE Chromaticity Diagram that lie on a curve that is between 0.035 Duv below and 0.035 above a planckian locus defined by the ANSI C78.377-2015 color standard). As a result, the combined or blended light output by the light fixture 500 is aesthetically pleasing to humans, as illustrated in, for example, FIG. 14E.

With reference back to FIG. 14D, the lighting device 504 also includes a first LED driver 564 and a second LED driver 568 each electrically connected to the controller 520 and powered by external power (e.g., AC power) received from an external power source (not shown). Responsive to instructions or commands received from the controller 520, the first LED driver 564 is configured to power the first LEDs 556, while the second LED driver 568 is configured to power the second LEDs 560. In other examples, the lighting device 564 can include more or less LED drivers. As an example, the lighting device 564 can include only one LED driver, configured to power the first LEDs 556 and the second LEDs 560, or can include multiple LED drivers configured to power the first LEDs 556 and multiple LED drivers configured to power the second LEDs 560.

As also illustrated in FIG. 14D, the controller 520 may receive a dimmer setting 572 and/or a mode control setting 576 received from a user of the lighting device 504 (e.g., input via a dimming switch electrically connected to the light fixture 500) and/or a central controller via, e.g., the server 66. The dimmer setting 572 is a 0-10 V control signal that specifies the desired dimmer or dimming level for the lighting device, which is a ratio of a desired combined light output of the first and second LEDs 556, 560 to the maximum combined light output of the first and second LEDs 556, 560 (and which corresponds to the blended or combined output discussed above). The 0 V input generally corresponds to a desired dimming level of 100% (i.e., no power is supplied to the first LEDs 556 or the second LEDs 560), the 5 V input generally corresponds to a desired dimming level of 50%, and the 10 V input generally corresponds to a desired dimming level of 0% (i.e., the first and second LEDs 556, 560 are fully powered), though this need not be the case. The mode control setting 576 is a control signal that specifies the desired operating mode for the lighting device 504. The mode control setting 576 may, for example, specify that the lighting device 504 be in a first mode (e.g., an examination mode, a disinfection mode, a blended mode), whereby the first and second LEDs 556, 560 are fully powered, or a second mode (e.g., a nighttime mode), whereby the second LEDs 560 are powered while the first LEDs 556 are not powered (or are powered at a lower level). Other modes and/or modes corresponding to different power settings or levels may be utilized.

In operation, the light fixture 500 provides or outputs (e.g., emits) light based on or in response to commands or instructions from the local controller 520. More specifically, the first LED driver 564 and/or the second LED driver 568 power the first LEDs 556 and/or the second LEDs 560, such that the first LEDs 556 and/or the second LEDs 560 provide or output (e.g., emit) a desired level of light, based on or in response to commands or instructions to that effect received from the local controller 520. These commands or instructions may be generated based on or responsive to receipt of the dimmer setting 572, receipt of the mode control setting 576, occupancy data obtained by the occupancy sensor and/or daylight data obtained by the daylight sensor, and/or based on or responsive to commands or instructions received from the server 66 and/or the client devices 70. Thus, the light fixture 500, and more particularly the first LEDs 556 and/or the second LEDs 560, may provide (e.g., emit) light responsive to occupancy data obtained by the occupancy sensor, daylight data obtained by the daylight sensor, and/or other commands or instructions (e.g., timing settings, dimmer settings, mode control settings).

The light fixture 500 can, for example, responsive to data indicating that the environment 100 is occupied, data indicating that there is a more than pre-determined amount of natural light in the environment 100 (i.e., it is daytime), and/or various commands and instructions, emit light from the first LEDs 556 and the second LEDs 560, thereby producing a blended or combined output of white visible light discussed above. In turn, the light fixture 500 produces a visible white light that effectively inactivates dangerous pathogens (e.g., viruses and/or bacteria) in the environment 100, and, at the same time, illuminates the environment 100 in a safe and objectionable manner (e.g., because the environment 100 is occupied, it is daytime, and/or for other reasons).

However, responsive to data indicating that the environment 100 is not occupied or has been unoccupied for a pre-determined amount of time (e.g., 30 minutes, 60 minutes), the light fixture 500 can reduce the power of the second LEDs 560, such that a substantial portion of the output light is from the first LEDs 556, or shut off the second LEDs 560 (which are no longer needed to produce a visually appealing blended output since the environment 100 is unoccupied), such that light is only emitted from the first LEDs 556, as illustrated in FIG. 14F. The light fixture 500 can, at the same time, increase the power or intensity of the first LEDs 556 and, in some cases, can activate one or more third LEDs that are not shown but are configured, like the LEDs 556, to emit sufficiently high levels of specially configured visible light, in this case light having a wavelength in a range of between approximately 400 nm and approximately 420 nm (e.g., 405 nm). In this manner, the inactivation effectiveness of the light fixture 500 can be increased (without sacrificing the visual appeal of the light fixture 500, as the environment 100 is unoccupied) and, at the same time, the energy consumption of the light fixture 500 can be reduced, or at the very least maintained (by virtue of the first LEDs 556 being reduced or shut off).

In some cases, the light fixture 500 can, responsive to data indicating that the environment 100 is not occupied or has been unoccupied for a period of time less than a pre-determined amount of time (e.g., 30 minutes), provide or output the combined or blended light output (of the first and second LEDs 556, 560) discussed above. This provides a fail-safe mode that ensures that the environment 100 is indeed vacant before the second LEDs 560 are shut off or reduced.

The light fixture 500 can respond in a similar or different manner to data indicating that there is more than a pre-determined amount of natural light in the environment 100, such that there is no need for the light from the second LEDs 560, or there is less than a pre-determined amount of natural light in the environment 100 (i.e., it is nighttime, such that the environment 100 is unlikely to be occupied). If desired, the light fixture 500 may only respond in this manner responsive to data indicating that the environment 100 is unoccupied and data indicating that it is nighttime. Alternatively, the light fixture 500 may only respond in this manner responsive to timer settings (e.g., it is after 6:30 P.M.) and/or other commands or instructions.

The light fixture 500, and more particularly the first LEDs 556 and the second LEDs 560, can also be controlled responsive to settings such as the dimmer setting 572 and the mode control setting 576 received by the controller 520. Responsive to receiving the dimmer setting 572 or the mode control setting 576, the controller 520 causes the first and second LED drivers 564, 568 to power (or not power) the first and second LEDs 556, 560, respectively, in accordance with the received setting. More specifically, when the controller 520 receives the dimmer setting 572 or the mode control setting 576, the controller 520 instructs the first LED driver 564, via a first LED control signal 580, and instructs the second LED driver 568, via a second LED control signal 584, to power (or not power) the first and second LEDs 556, 560 according to the desired dimming level specified by the dimmer setting 572 or the desired operating mode specified by the mode control setting 576.

FIG. 14G illustrates one example of how the controller 520 can control the first and second LED drivers 564, 568 responsive to various dimmer settings 572 that specify various dimming levels (e.g., 0%, 25%, 50%, 75%, 100%). Generally speaking, the controller 520 causes the first and second LED drivers 564, 568 to increase the total light output by the first and second LEDs 556, 560 responsive to decreasing dimming levels, thereby increasing the color temperature of the total light output, and causes the first and second LED drivers 564, 568 to decrease the total light output by the first and second LEDs 556, 560 responsive to increasing dimming levels, thereby decreasing the color temperature of the total light output. But, as shown in FIG. 14G, the controller 520 controls the first LEDs 556 (via the first LED driver 564) differently than it controls the second LEDs 560 (via the second LED driver 568). In other words, there exists a non-linear relationship between the amount of light emitted by the first LEDs 556 and the amount of light emitted by the second LEDs 560 at various dimming levels. This relationship is illustrated by the fact that a first curve 588, which represents the total power supplied to the first and second LEDs 556, 560 by the first and second LED drivers 564, 568, respectively, as a function of various dimmer levels, is not parallel to or with a second curve 592, which represents the power supplied to the first LEDs 556 as a function of the same varying dimmer levels. As an example, (i) when the dimmer setting 572 specifies a dimmer level of 0% (i.e., no dimming), such that the light fixture 500 is operated at full (100%) power, approximately 50% of that total power is supplied to the first LEDs 556, (ii) when the dimmer setting 572 specifies a dimmer level of 50%, such that the light fixture 500 is operated at half (50%) power, less than 50% of that total power is supplied to the first LEDs 556, and (iii) when the dimmer setting 572 specifies a dimmer level of greater than 75% but less than 100%, such that the light fixture 500 is operated at a power less than 25%, no power is supplied to the first LEDs 556. As a result, the first LEDs 556 are turned completely off before the second LEDs 560 are turned completely off. In this manner, the light output by the light fixture 500 remains unobjectionable and aesthetically pleasing, even while the light fixture 500 is dimmed, particularly when dimmed to very high levels (e.g., 80%, 85%, 90%, 95%).

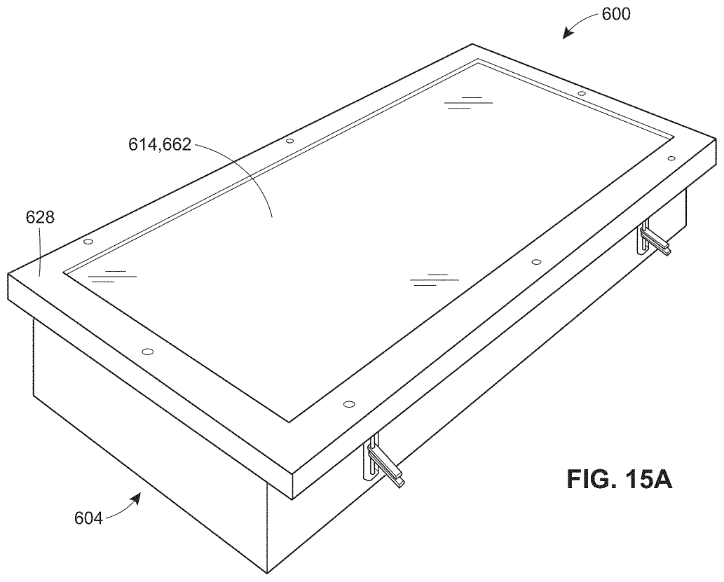

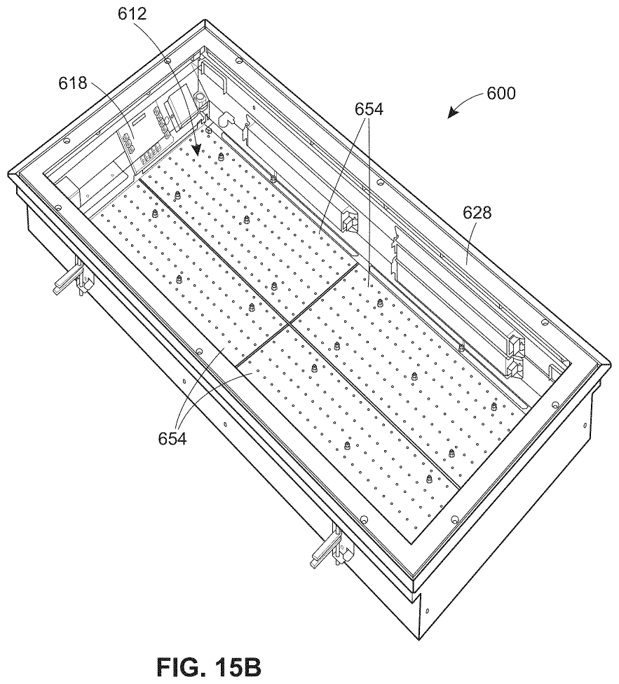

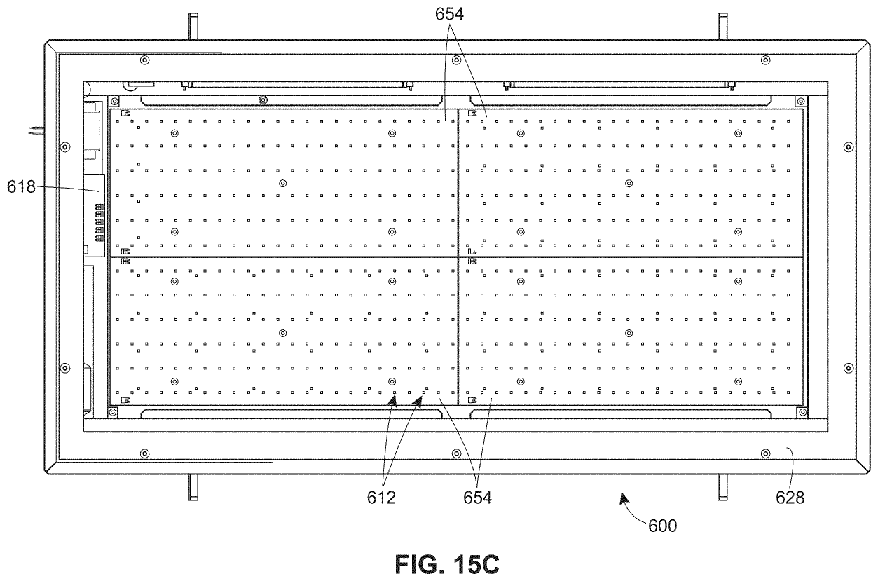

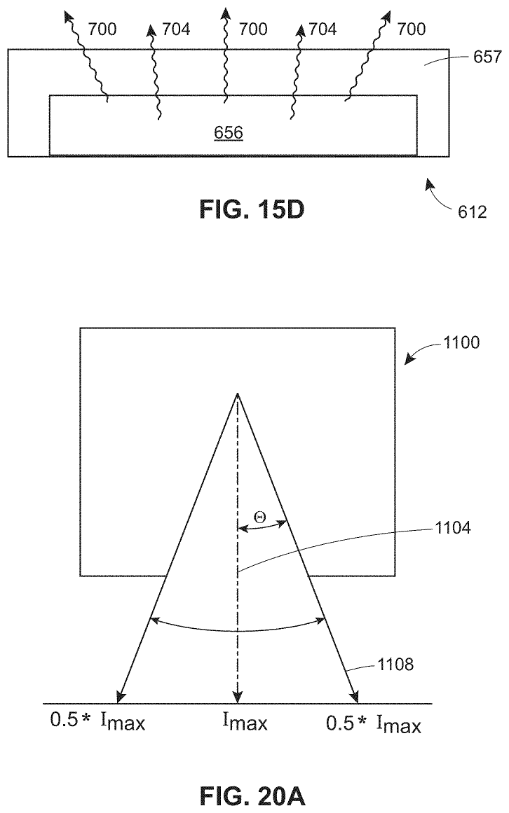

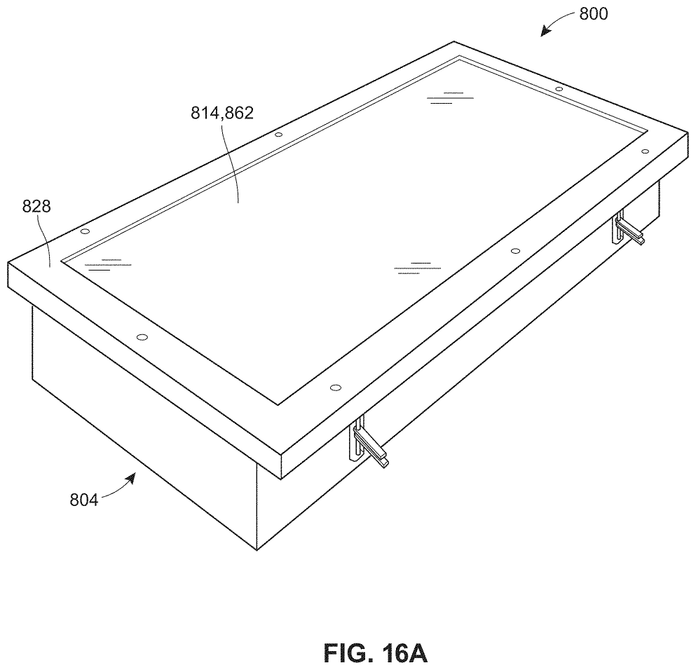

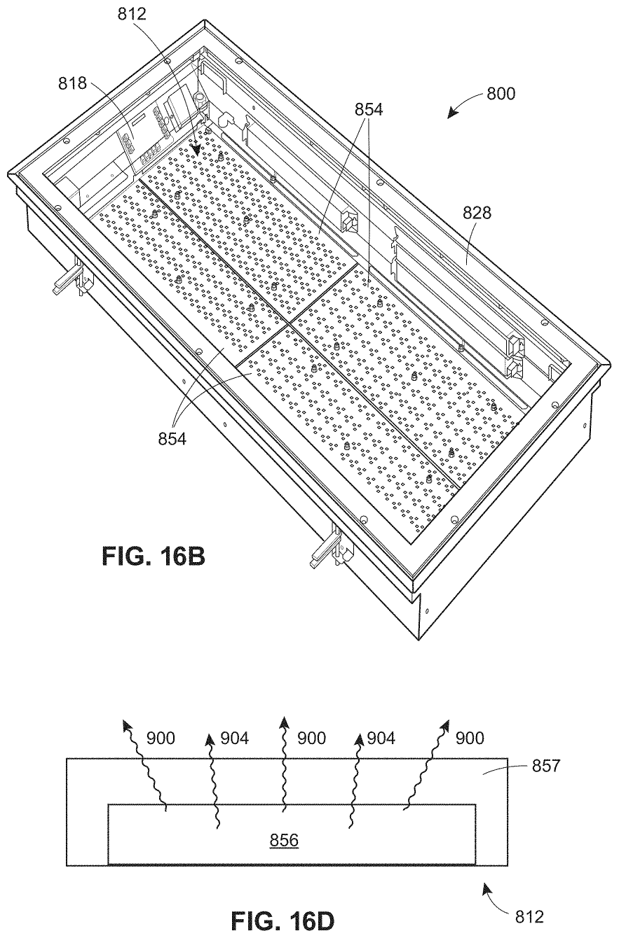

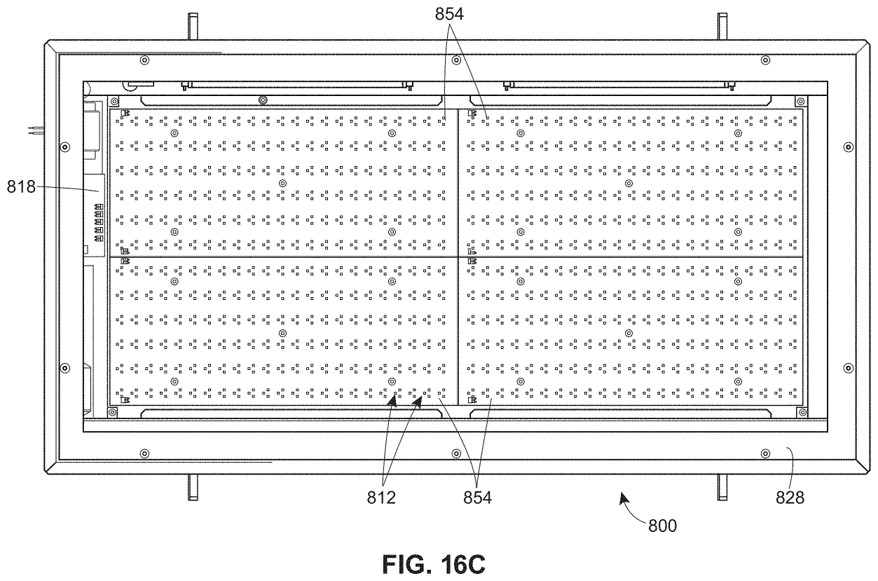

FIGS. 15A-15D illustrate yet another version of the lighting device 104, in the form of a light fixture 600. The light fixture 600 is similar to the light fixture 500 in that it includes a housing or chassis 604 (with a flange 628) and a lens 614 configured to diffuse light emitted by the light fixture in an efficient manner, as well as components like a local controller, an occupancy sensor, a communication module, and a dosing feedback system identical to the controller 120, the sensor 108, the module 116, and the dosing feedback system 124, respectively, described above; thus, for the sake of brevity, these components will not be described in any further detail. The light fixture 600 may also include any of the means for maintaining junction temperature discussed above in connection with the lighting device 104. However, the light fixture 600 includes a plurality of lighting elements 612 that is different from the plurality of light emitting elements 512 of the light fixture 500. While the lighting elements 612 are, like the elements 512, arranged on LED modules 654 in a sealed or closed light-mixing chamber defined by the housing 604 and the lens 614, as illustrated in FIGS. 15B and 15C, each of the lighting elements 612 takes the form of a light-emitting diode (“LED”) 656 and a light-converting element 657 that is associated therewith and is configured to convert a portion of the light emitted by the LED 656, as illustrated in FIG. 16D. In this version, each LED module 654 includes seventy-six (76) lighting elements 612, though in other versions, more or less lighting elements 612 can be employed (and/or additional LEDs 656 can be employed without light-converting elements 657). In this version, the light-converting element 657, which may for example be a phosphor element such as a phosphor or a substrate infused with phosphor, covers or coats the LED 656, though in other versions the light-converting element 657 may be located remotely from the LED 656 (e.g., a remote phosphor element).

In operation, the LEDs 656 of the lighting elements 612 emit disinfecting light (e.g., light having a wavelength of between 400 nm and 420 nm, such as 405 nm) that, when combined or summed, produces power levels sufficient to inactivate pathogens. As discussed above, the LEDs 656 may combine to emit at least 3,000 mW of the disinfecting light, e.g., 3,000 mW, 4,000 mW, 5,000 mW, or some other level of visible light above 3,000 mW. At least a first portion or component 700 (and in FIG. 15D, multiple components 700) of the disinfecting light emitted by each LED 656 travels or passes through the respective light-converting element 657 without alteration, while at least a second portion or component 704 (and in FIG. 15D, multiple components 704) of the disinfecting light emitted by each LED 656 is (are) converted by the respective light-converting element 657 into light having a wavelength of greater than 420 nm. In many cases, the second portion(s) or component(s) 704 of light is (are) converted into yellow light, i.e., light having a wavelength of between 570 nm and 590 nm. In other words, each lighting element 612 is configured to provide light, at least a first component of the light, provided by the respective LED 656, having a wavelength of between 400 nm and 420 nm (e.g., 405 nm) and at least a second component of the light, provided by the respective light-converting element 657, having a wavelength of greater than 420 nm. The first component(s) of the provided light will, as is also described above, have a minimum irradiance, measured, at least in this example, from any exposed surface or unshielded point in the environment 100 that is 1.5 m from any point on any external-most luminous surface 662 of the lighting device 504, equal to a value between 0.01 mW/cm² and 10 mW/cm², or preferably, between 0.01 mW/cm² and 1.0 mW/cm², as irradiance values above 1.0 mW/cm² are likely to exceed the exposure limit prescribed by the IEC 62471 standard. More particularly, the first component(s) of the provided light will have a minimum irradiance equal to a value between 0.035 mW/cm² and 0.6 mW/cm², in view of the considerable virucidal effects of these irradiances as demonstrated in the studies described herein. The minimum irradiance may, for example, be equal to 0.01 mW/cm², 0.02 mW/cm², 0.035 mW/cm², 0.05 mW/cm², 0.076 mW/cm², 0.1 mW/cm², 0.15 mW/cm², 0.20 mW/cm², 0.25 mW/cm², 0.30 mW/cm², 0.35 mW/cm², 0.40 mW/cm², 0.45 mW/cm², 0.50 mW/cm², 0.55 mW/cm², 0.60 mW/cm², 0.65 mW/cm², 0.70 mW/cm², 0.75 mW/cm², 0.80 mW/cm², 0.85 mW/cm², 0.90 mW/cm², 0.95 mW/cm², 1.00 mW/cm², or some other value in the above-specified ranges. In other examples, the minimum irradiance can be measured from a different distance from any point on any external-most luminous surface 662, nadir, or some other exposed surface or point in the environment 100.

At the same time, the light provided or output by the light fixture 600, and more particularly each lighting element 612, is a white light having the properties discussed above, such that the provided light is aesthetically pleasing, or at least unobjectionable, to humans. This is because the light provided by the light converting elements 657, i.e., the second component(s), complements the disinfecting light that is emitted by the LEDs 656 and passes through the light converting elements 657 without alteration, i.e., the first component(s).

As with the light fixture 500, the light fixture 600 can provide or output light based on or in response to commands or instructions from a local controller 618. These commands or instructions may be generated based on or responsive to occupancy data obtained by the occupancy sensor and/or daylight data obtained by the daylight sensor, and/or based on or responsive to commands or instructions received from a user of the light fixture 600 (e.g., via the client devices 70) and/or the server 66. Thus, the light fixture 600 may provide light responsive to occupancy data obtained by the occupancy sensor, daylight data obtained by the daylight sensor, and/or other commands or instructions (e.g., timing settings).

FIGS. 16A-16D illustrate yet another version of the lighting device 104, in the form of a light fixture 800. The light fixture 800 is similar to the light fixture 600 in that it includes a housing or chassis 804 (with a flange 628) and a lens 814 configured to diffuse light emitted by the light fixture in an efficient manner, as well as components like a local controller, an occupancy sensor, a communication module, and a dosing feedback system identical to the controller 120, the sensor 108, the module 116, and the dosing feedback system 124, respectively, described above; for the sake of brevity, these components will not be described in any further detail. The light fixture 800 may also include means, such as support arms like the support arms 516 described above, for mounting the housing 804 to a surface (e.g., a ceiling, a floor, a wall) in the environment 100, and/or include any of the means for maintaining junction temperature discussed above in connection with the lighting device 104.

However, the light fixture 800 includes a plurality of lighting elements 812 that is different from the plurality of light emitting elements 612 of the light fixture 600. Like the elements 612, the lighting elements 812 are arranged on LED modules 854 in a sealed or closed light-mixing chamber defined by the housing 804 and the lens 814, as illustrated in FIGS. 16B and 16C, and each of the lighting elements 812 takes the form of a light-emitting diode (“LED”) 856 and a light-converting element 857 that is associated therewith and is configured to convert a portion of the light emitted by the respective LED 856, as illustrated in FIG. 16D. But unlike the elements 612, the lighting elements 812 are arranged in clusters 884. Each of the clusters 884 generally includes a subset of the overall total number of lighting elements 812 in the light fixture 800. In this version, each of the clusters 884 includes three LEDs 856 configured to emit disinfecting light (e.g., light having a wavelength of between 400 nm and 420 nm, a wavelength of between 460 nm and 480 nm) and three light-converting elements 857, in the form of three phosphor elements, that cover or coat the respective LEDs 856 and convert a portion of the disinfecting light emitted by the LEDs 856 into disinfecting light of a different wavelength (or different wavelengths) than the disinfecting light emitted by the LEDs 856. As an example, each of the clusters 884 may include three LEDs 856 configured to emit disinfecting light having a wavelength of between 400 nm and 420 nm (e.g., about 405 nm) and three different phosphor elements, a blue phosphor that converts a portion of the disinfecting light emitted by one of the LEDs 856 into disinfecting light having a wavelength of between 460 nm and 480 nm, a green phosphor that converts a portion of the disinfecting light emitted by another one of the LEDs 856 into disinfecting light having a wavelength of between 530 nm and 580 nm, and a red phosphor that converts a portion of the disinfecting light emitted by the remaining LED 856 into disinfecting light having a wavelength of between 600 nm and 650 nm. In other versions, however, the lighting elements 812 need not be arranged in clusters 884 or can be arranged in different clusters 884. More particularly, the clusters 884 may include a different number of LEDs 856 (e.g., additional LEDs 856 can be employed without light-converting elements 857), a different number of light-converting elements 857, different LEDs 856, or different light-converting elements 857. As an example, the light-converting elements 857 may be located remotely from the LEDs 856 or the light-converting elements 857 may instead take the form of a quantum dot or other means for converting light in the described manner.

In operation, the LEDs 856 of the lighting elements 812 emit disinfecting light (e.g., light having a wavelength of between 400 nm and 420 nm). At least a first portion or component 900 (and in FIG. 16D, multiple components 900) of the disinfecting light emitted by each LED 856 travels or passes through the respective light-converting element 857 without alteration, while at least a second portion of component 904 (and in FIG. 16D, multiple components 904) of the disinfecting light emitted by each LED 856 is (are) converted by the respective light-converting element 857 into disinfecting light having a different wavelength than the wavelength of the disinfecting light emitted by the respective LED 856. In other words, each lighting element 812 is configured to provide disinfecting light, at least a first component of which is provided by the respective LED 856 and at least a second component of which is provided by the respective light-converting element 857. As discussed above, the first and/or second component(s) of the disinfecting light may have a minimum irradiance, measured, at least in this example, from any exposed surface or unshielded point in the environment 100 that is 1.5 m from any point on any external-most luminous surface 862 of the lighting device 804, equal to a value between 0.01 mW/cm² and 10 mW/cm², or preferably, between 0.01 mW/cm² and 1.0 mW/cm², as irradiance values above 1.0 mW/cm² are likely to exceed the exposure limit prescribed by the IEC 62471 standard. More particularly, the first component(s) of the provided light may have a minimum irradiance equal to a value between 0.035 mW/cm² and 0.6 mW/cm², in view of the considerable virucidal effects of these irradiances as demonstrated in the studies described herein. The minimum irradiance may, for example, be equal to 0.01 mW/cm², 0.02 mW/cm², 0.35 mW/cm², 0.05 mW/cm², 0.76 mW/cm², 0.1 mW/cm², 0.15 mW/cm², 0.20 mW/cm², 0.25 mW/cm², 0.30 mW/cm², 0.35 mW/cm², 0.40 mW/cm², 0.45 mW/cm², 0.50 mW/cm², 0.55 mW/cm², 0.60 mW/cm², 0.65 mW/cm², 0.70 mW/cm², 0.75 mW/cm², 0.80 mW/cm², 0.85 mW/cm², 0.90 mW/cm², 0.95 mW/cm², 1.00 mW/cm², or some other value in the above-specified ranges. In other examples, the minimum irradiance can be measured from a different distance from any point on any external-most luminous surface 862, nadir, or some other exposed surface or point in the environment 100. In any case, because the first component(s) and the second component(s) are, on their own, sufficient to inactivate pathogens in the environment 100, the first and second components of the disinfecting light, when combined or summed, produce disinfecting doses more than sufficient to inactivate pathogens in the environment 100. While the exact disinfecting energy achieved by the combination of the first and second components will vary depending upon the exact application, the combined light has a disinfecting energy, measured, at least in this example, from any unshielded point (e.g., air or surface) in the environment 100, equal to at least 0.06 J/cm².

At the same time, the disinfecting light emitted by the light-converting elements 857 (i.e., the second components) complements the disinfecting light emitted by the LEDs 856, such that the combined or blended light output formed in the mixing chamber of the fixture 800 is a non-white light having the properties discussed above (e.g., non-white light having u′, v′ coordinates on the 1976 CIE Chromaticity Diagram that lie outside of an area that is bounded (i) vertically between the curve 106A and the curve 1066, a curve 109A that is 0.007 Duv above the planckian locus 105 and a curve 1096 that is 0.007 Duv below (−0.007 Duv) the planckian locus 105, or other curves, and (ii) horizontally between a color temperature isoline of between approximately 1500 K and 7000 K). As a result, the combined or blended light output by the light fixture 800 is aesthetically pleasing, or at least unobjectionable, to humans in the environment 100.

As with the light fixtures 500 and 600, the light fixture 800 can provide or output light based on or in response to commands or instructions from a local controller 818. These commands or instructions may be generated based on or responsive to occupancy data obtained by the occupancy sensor and/or daylight data obtained by the daylight sensor, and/or based on or responsive to commands or instructions received from a user of the light fixture 800 (e.g., via the client devices 70) and/or the server 66. Thus, the light fixture 800 may provide light responsive to occupancy data obtained by the occupancy sensor, daylight data obtained by the daylight sensor, and/or other commands or instructions (e.g., timing settings).

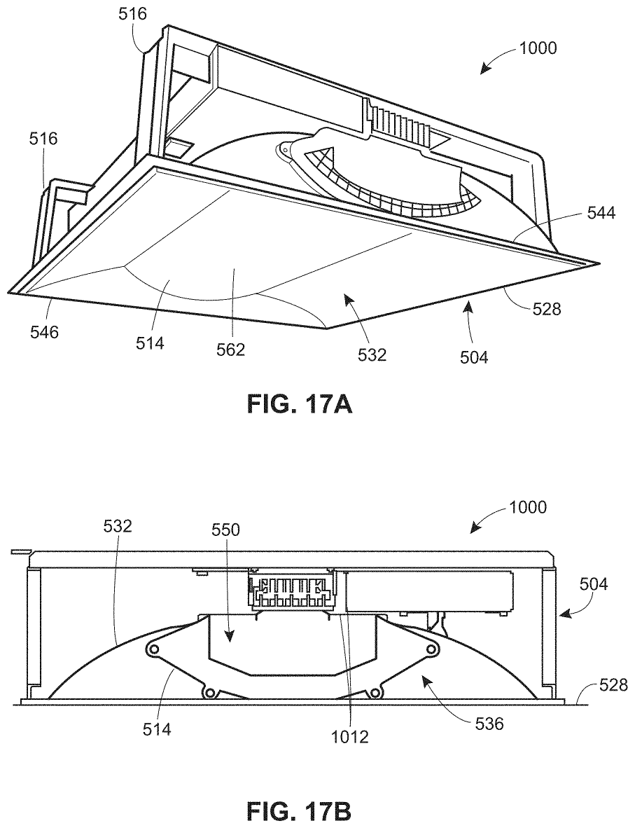

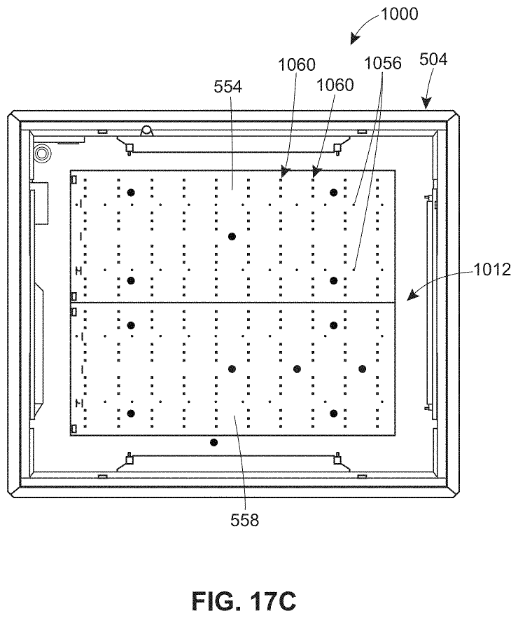

FIGS. 17A-17C illustrate yet another version of the lighting device 104, in the form of a light fixture 1000. The light fixture 1000 is similar to the light fixture 500, with common reference numerals used for common components, but includes a plurality of light-emitting elements 1012 different from the plurality of light-emitting elements 512. The light fixture 1000 is similar to the light fixture 500 in that the plurality of light-emitting elements 1012 also take the form of a plurality of first LEDs 1056 and a plurality of second LEDs 1060, and the first LEDs 1056 are, like the first LEDs 556, configured to provide (e.g., emit) disinfecting light having a wavelength between 400 nm and 420 nm (e.g., light having a wavelength of about 405 nm). However, the first LEDs 1056 together contribute less power to the total power level of light provided by the light fixture 1000 than the first LEDs 556 together contribute to the total power level of light provided by the light fixture 500. In some cases, this will be achieved by including less first LEDs 1056 in the fixture 1000 (as compared to the number of LEDs 556 included in the fixture 500). In other cases, this may be achieved by varying the total power provided by the first LEDs 1056 via, for example, a controller.

In any case, having the first LEDs 1056 contribute less power removes some 400 nm to 420 nm disinfecting light from the overall light output by the light fixture 1000, to ensure comfort and safety for occupants of the environment 100. In turn, the first LEDs 1056 generally combine to provide (e.g., emit) less levels of disinfecting light than the first LEDs 556. Thus, for example, the minimum irradiance of the disinfecting light provided by all of the LEDs 1056 is generally less than the minimum irradiance of the disinfecting light provided by all of the LEDs 556. Nonetheless, the minimum irradiance of the disinfecting light provided by all of the LEDs 1056, measured, at least in this example, from any exposed surface or unshielded point in the environment 100 that is 1.5 m from any point on any external-most luminous surface 562 of the fixture 1000, may be equal to a not insignificant value such as 0.01 mW/cm², 0.02 mW/cm², 0.035 mW/cm², 0.05 mW/cm², 0.076 mW/cm², 0.1 mW/cm², 0.15 mW/cm², 0.20 mW/cm², 0.25 mW/cm², 0.30 mW/cm², 0.35 mW/cm², 0.40 mW/cm², 0.45 mW/cm², 0.50 mW/cm², 0.55 mW/cm², 0.60 mW/cm², 0.65 mW/cm², 0.70 mW/cm², 0.75 mW/cm², 0.80 mW/cm², 0.85 mW/cm², 0.90 mW/cm², 0.95 mW/cm², 1.00 mW/cm², or some other value between 0.01 mW/cm² and 10 mW/cm², or preferably, between 0.01 mW/cm² and 1.0 mW/cm² (or still more particularly, between 0.035 mW/cm² and 0.6 mW/cm²).

In order to ensure that the light fixture 1000 provides sufficiently high levels of disinfecting light so as to inactivate pathogens in the environment 100, the second LEDs 1060 are, unlike the second LEDs 560, also configured to provide (e.g., emit) disinfecting light, albeit disinfecting light having a wavelength that is different from the wavelength of the light emitted by the first LEDs 1056. For example, the second LEDs 1060 can be configured to provide disinfecting light having a wavelength of between 460 nm to 480 nm, light having a wavelength of 530 nm to 580 nm, or light having a wavelength of between 600 nm and 650 nm. The minimum irradiance of the disinfecting light provided by all of the second LEDs 1060 may be greater than, less than, or equal to the minimum irradiance of the disinfecting light provided by all of the first LEDs 1056, but generally falls within the range discussed above. Additionally, in some cases, the plurality of light-emitting elements 1012 may also additional LEDs (e.g., a plurality of third LEDs) to provide additional disinfecting light having a wavelength that is different from the wavelengths of the light emitted by the first and second LEDs 1056, 1060 and/or to provide visible light when necessary to complement the light provided by the first and second LEDs 1056, 1060.

Accordingly, the combination of the disinfecting light provided by the first LEDs 1056 and the second LEDs 1060 (and any additional LEDs, when utilized) produces disinfecting doses more than sufficient to inactivate pathogens in the environment 100. While the exact disinfecting energy achieved by this combination will vary depending upon the exact application, the combined light has a disinfecting energy, measured, at least in this example, from any unshielded point (e.g., air or surface) in the environment 100, equal to at least 0.06 J/cm².

At the same time, by substituting some of the disinfecting light having a wavelength of between 400 nm to 420 nm with disinfecting light of other wavelengths, and by providing disinfecting light of other wavelengths via the second LEDs 1060 that complements the disinfecting light provided by the first LEDs 1056, the combined or blended light output by the fixture 1000 is an unobjectionable non-white light having the properties discussed above (e.g., non-white light having u′, v′ coordinates on the 1976 CIE Chromaticity Diagram that lie outside of an area that is bounded (i) vertically between the curve 106A and the curve 1066, a curve 109A that is 0.007 Duv above the planckian locus 105 and a curve 1096 that is 0.007 Duv below (−0.007 Duv) the planckian locus 105, or other curves, and (ii) horizontally between a color temperature isoline of between approximately 1500 K and 7000 K).

As with the light fixtures 500 and 600, the light fixture 1000 can provide or output light based on or in response to commands or instructions from a local controller. These commands or instructions may be generated based on or responsive to occupancy data obtained by the occupancy sensor and/or daylight data obtained by the daylight sensor, and/or based on or responsive to commands or instructions received from a user of the light fixture 1000 (e.g., via the client devices 70) and/or the server 66. Thus, the light fixture 1000 may provide light responsive to occupancy data obtained by the occupancy sensor, daylight data obtained by the daylight sensor, and/or other commands or instructions (e.g., timing settings).

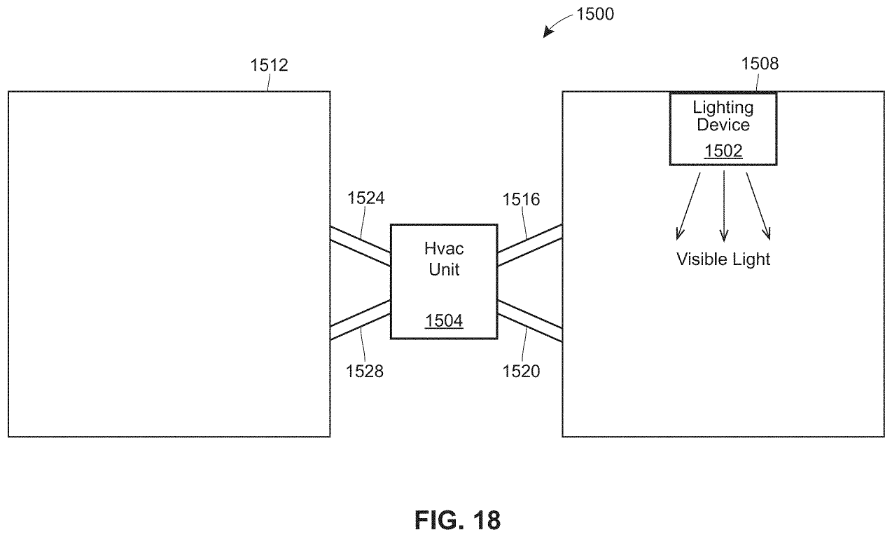

FIG. 18 illustrates a healthcare environment 1500 that includes a lighting device 1502, in the form of one of the lighting devices described herein (e.g., the lighting device 1000), employed in conjunction with an HVAC unit 1504 for the healthcare environment 1500. In this version, the healthcare environment 1500 includes a first room 1508 (e.g., an operating room, a waiting room, an examination room) and a second room 1512 (e.g., an operating room, a waiting room, an examination room) that is structurally separate from the first room 1512 but shares the HVAC unit 1504 with the first room 1508. In other versions, however, the healthcare environment 1500 may include a different number of rooms (e.g., one room, three or more rooms, etc.) Further, in this version, the first room 1508 includes the lighting device 1502 but the second room 1512 does not include any of the lighting devices described herein. However, in other versions, the first room 1508 may include more than one lighting device 1502 and/or the second room 1508 may include one or more of the lighting devices described herein (in which case the first room 1508 may not include the lighting device 1502).

The HVAC unit 1504 is generally configured to provide air (e.g., Class 1, Class 10, Class 100, Class 1,000, Class 10,000, or Class 100,000 air) to the healthcare environment 1500. To this end, the HVAC unit 1504 is connected to the first room 1508 via a first supply air duct 1516 and a first return air duct 1520, and to the second room 1512 via a second supply air duct 1524 and a second return air duct 1528. The HVAC unit 1504 may, via the air ducts 1516, 1520, replace the air in the first room 1508, and, via the air ducts 1524, 1528, replace the air in the second room 1512; this can be done any number of times per hour (e.g., 3, 8, 40 times per hour). In some cases, e.g., when the healthcare environment 1500 is part of a larger environment (e.g., a hospital), the HVAC unit 1504 may be connected to a central HVAC system. In other cases, the HVAC unit 1504 may itself be considered the central HVAC system.

In operation, the HVAC unit 1504 provides (e.g., delivers) air to the first room 1508 via the first supply air duct 1516 and to the second room 1512 via the second supply air duct 1520. In turn, the lighting device 1502, which provides disinfecting light as discussed above, inactivates pathogens in the air (i.e., disinfects the air) provided to the first room 1508 and proximate the lighting device 1502. The air in the first room 1508 is continuously circulated, such that the disinfected air is moved away from the lighting device 1502 and air that has not yet been disinfected is moved into proximity of the lighting device 1502 and disinfected. The air in the first room 1508 circulates in this manner because of a natural air convection current created by the temperature difference between the ambient temperature in the environment 1500 and the surface temperature of the outermost surface of the lighting device 1502, which will be greater than the ambient temperature, in the vicinity of the lighting device 1502. Optionally, additional air convection may be created by incorporating one or more fans, one or more heat sinks, and/or one or more other physical means for creating additional air convection into or onto the lighting device 1502.

Over time, the HVAC unit 1504 replaces the air originally provided to the first room 1508 with air originally provided to the second room 1512, and replaces the air originally provided to the second room 1512 with the air originally provided to the first room 1508 (and since substantially disinfected by the lighting device 1502). Thus, the HVAC unit 1504 also serves to circulate the air in the healthcare environment 1500 between the first room 1508 and the second room 1512, thereby ensuring that not only will substantially all of the air in the first room 1508 be disinfected, but that substantially all of the air in the healthcare environment 1500 is disinfected several times per hour (this number will largely be dictated by how often the HVAC unit 1504 changes the air in the environment 1500).

Studies performed by the Applicant on healthcare environments configured like the healthcare environment 1500 have shown that employing one or more lighting devices in accordance with the present disclosure in a first room of an environment (e.g., the first room 1508) not only significantly reduces the incidence of HAIs in occupants of that first room, but also significantly reduces the incidence of HAIs in occupants of a second room (e.g., the second room 1512), and other rooms, when those rooms utilize the same HVAC unit (e.g., the HVAC unit 1504). Thus, the Applicant has found that HAIs can be significantly reduced across healthcare environments without having to go to the (significant) expense of installing multiple disinfecting lighting devices in each of the rooms in that environment.

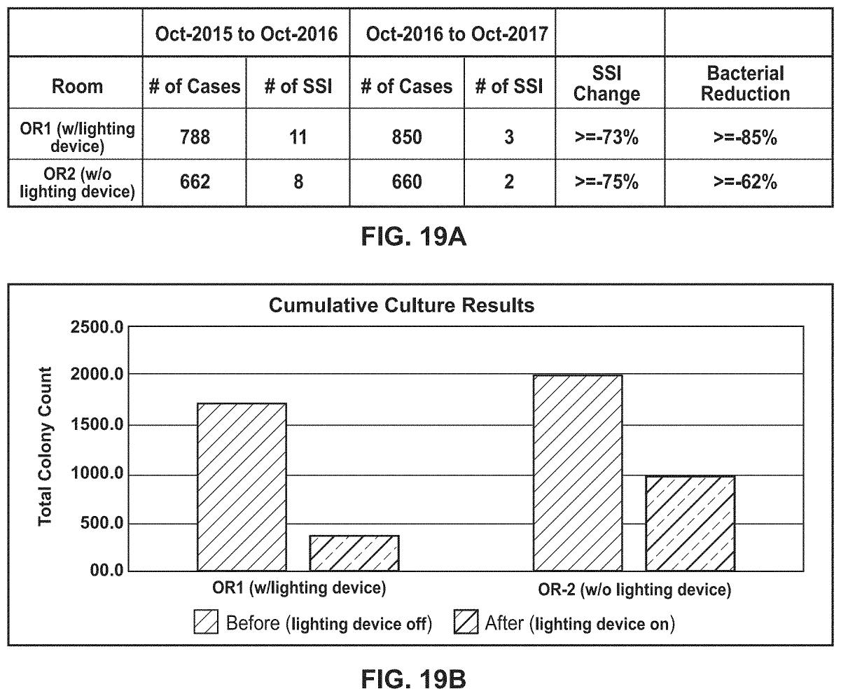

In one such study, a disinfecting lighting device constructed in accordance with the teachings of the present disclosure was installed in an orthopedic operating room OR1 at Maury Regional Health Center. Bacteria levels in the orthopedic operating room OR1 were subsequently measured for a period of 30 days and compared with bacteria levels measured in the orthopedic operating room OR1 prior to the installation of the lighting device therein. As illustrated in FIGS. 19A and 19B, the disinfecting lighting device reduced bacteria levels within the operating room OR1 by approximately 85%. Unexpectedly, during that same time period, the disinfecting lighting device also reduced lighting bacteria levels within an orthopedic operating room OR2 that is separate from but is adjacent to and shares an HVAC unit with the orthopedic operating room OR1 by approximately 62%. Infection rates for surgical site infections (SSIs), which are a subset of HAIs, for the operating room OR1 were also tracked for a 12 month period of time (October 2016 to October 2017) following the installation of the lighting device within the orthopedic operating room OR1 and compared to infection rates in the operating room OR1 for the 12 month period of time (October 2015 to October 2016) prior to the installation of the lighting device. As illustrated in FIG. 19A, the disinfecting lighting device installed in the operating room OR1 reduced the number SSIs by 73%. Unexpectedly, consistent with the data on bacteria reduction, the disinfecting lighting device also reduced the number of SSIs for the operating room OR2 (adjacent the operating room OR2) by 75%.

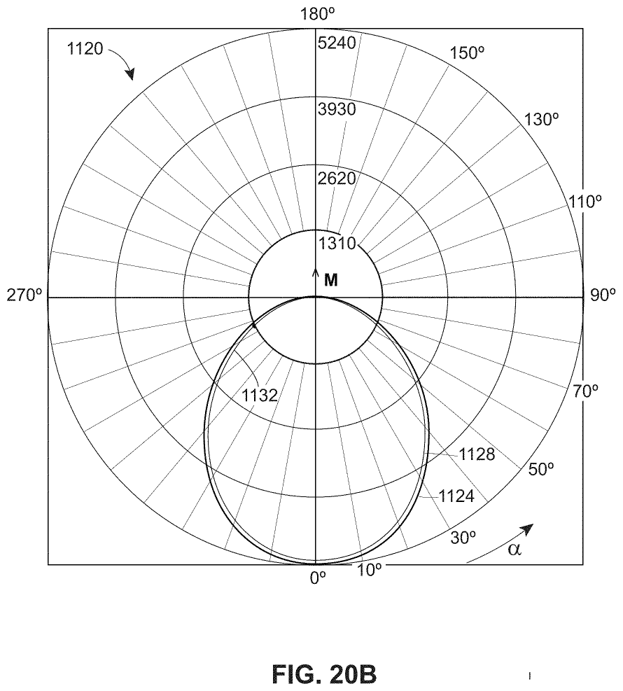

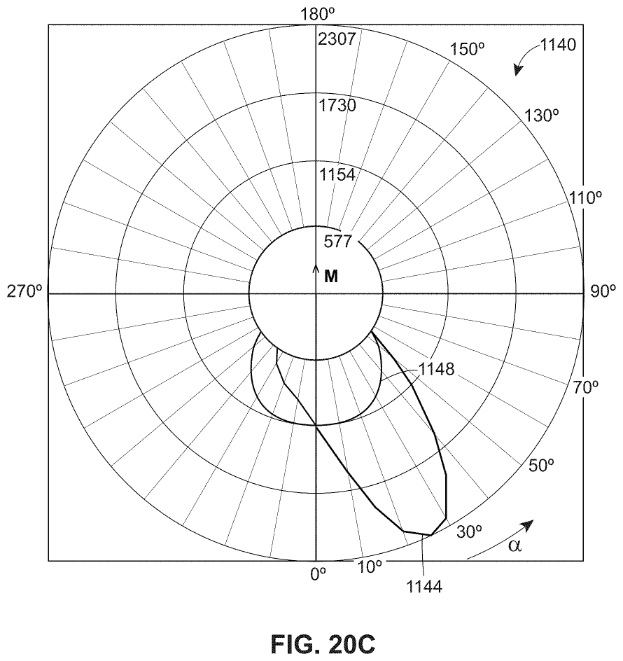

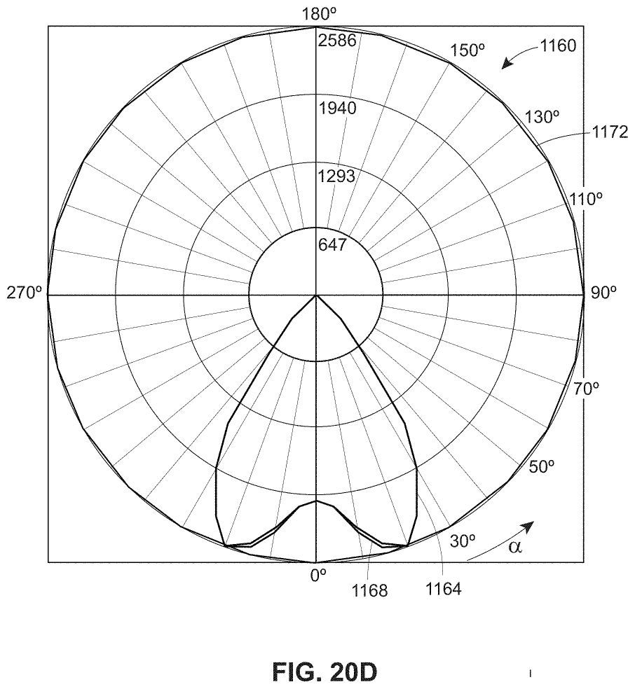

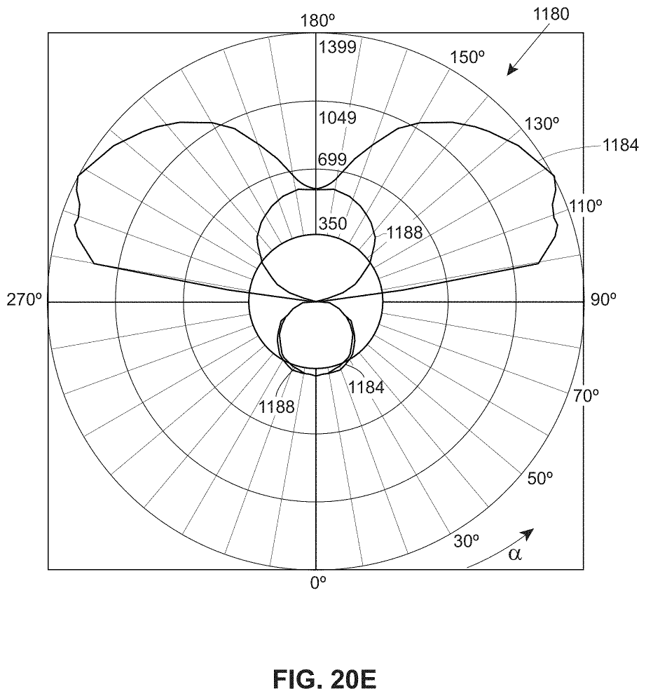

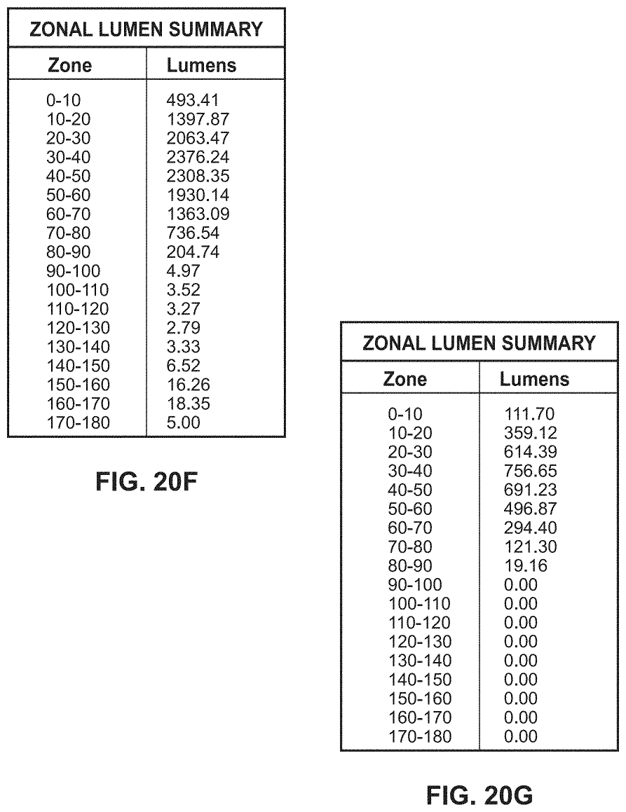

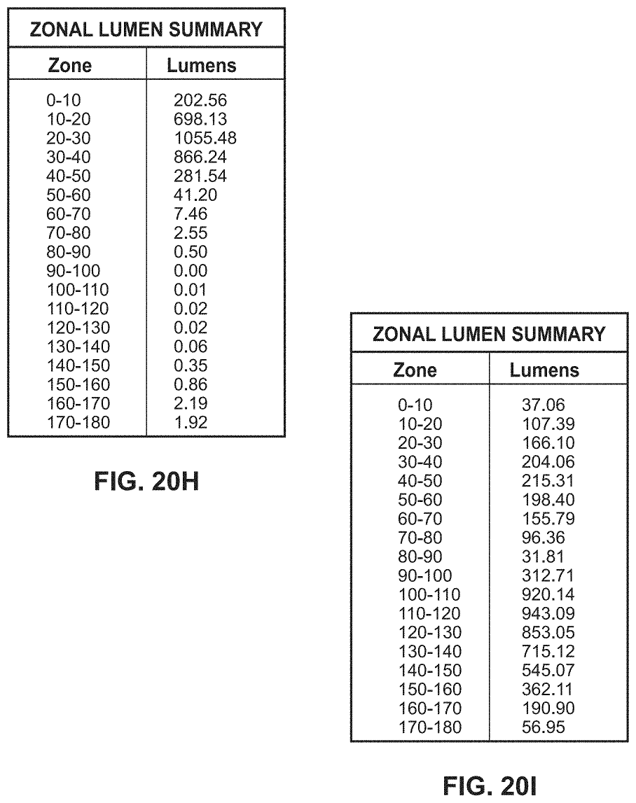

FIG. 20A illustrates one example of a distribution of the radiometric power output by a lighting device 1100, which takes the form of any one of the lighting devices 104, 200, 500, 600, 800, and 1000 described herein. As illustrated in FIG. 20A, the radiometric power is at a maximum value along a center axis 1104 of the light distribution from the lighting device 100, while the radiometric power along a line 1108 oriented at an angle θ from the center axis 1104 is equal to 50% of the maximum radiometric power value, so long as the radiometric power at the center axis 1104 and the radiometric power on the line 1108 are measured at equal distances from the lighting device 1100. The line 1108 in this version is oriented at an angle equal to 20 or 30 degrees from the center axis 1104, but may, in other versions, be oriented at a different angle θ.

It will be appreciated that a lighting device such as one of the lighting devices 104, 200, 500, 600, 800, 1000, and 1100 described herein can distribute light within or throughout the environment 100 in any number of different ways, depending upon the given application. The lighting device can, for example, utilize a lambertian distribution 1120, an asymmetric distribution 1140, a downlight with cutoff distribution 1160, or a direct-indirect distribution 1180, as illustrated in FIGS. 20B-20E, respectively.

The lambertian distribution plot 1120 illustrated in FIG. 20B takes the form of a two-dimensional polar graph that depicts a magnitude M of the intensity of the light output from a lighting device as a function of the vertical a from the horizontal. As shown in FIG. 20B, the lambertian distribution plot 1120 includes a first light distribution 1124 measured along a vertical plane through horizontal angles 0-180 degrees, a second light distribution 1128 measured along a vertical plane through horizontal angles 90-270 degrees, and a third light distribution 1132 measured along a vertical plane through horizontal angles 180-0 degrees. As illustrated by each of the first, second, and third light distributions 1124, 1128, and 1132, the magnitude M of light intensity is at its maximum value (in this example, 5240 candela) when the vertical angle a is equal to 0 degrees (i.e., nadir), such that the main beam angle, which corresponds to the vertical angle of highest magnitude, is equal to 0 degrees. The magnitude M then decreases as the vertical angle a moves from 0 degrees to 90 degrees.

The asymmetric distribution plot 1140 illustrated in FIG. 20C likewise takes the form of a two-dimensional polar graph that depicts the magnitude M of the intensity of the light output from a lighting device as a function of the vertical a from the horizontal. As shown in FIG. 20C, the asymmetric distribution plot 1140 includes a first light distribution 1144 measured along a vertical plane through horizontal angles between 0-180 degrees and a second light distribution 1148 measured along a vertical plane through horizontal angles between 90-270 degrees. As illustrated by the first and second light distributions 1144, 1148, light is distributed asymmetrically to one side of the lighting device, with the magnitude M of light intensity at its maximum value (in this example, 2307 candela) when the vertical angle α is equal to 25 degrees, such that the main beam angle, which corresponds to the vertical angle a of highest magnitude, is equal to 25 degrees. Such a distribution may, for example, be utilized in an environment 100 that features an operating table, so that the main beams of light from the lighting device are directed toward the operating table.

The downlight with cutoff distribution plot 1160 illustrated in FIG. 20D also takes the form of a two-dimensional polar graph that depicts the magnitude M of the intensity of the light output from a recessed lighting device as a function of the vertical a from the horizontal. As shown in FIG. 20D, the distribution plot 1160 includes a first light distribution 1164 measured along a vertical plane through horizontal angles between 0-180 degrees, a second light distribution 1168 measured along a vertical plane through horizontal angles between 90-270 degrees, and a third light distribution 1172 measured along a horizontal cone through a vertical angle α of 20 degrees. As illustrated by the first, second, and third light distributions 1164, 1168, and 1172, the magnitude M of light intensity is at its maximum value (in this example, 2586 candela) when the horizontal angle is 60 degrees and the vertical angle α is equal to 20 degrees, and there is very minimal light intensity (i.e., the light is cutoff) above 45 degrees. The main beam angle, which corresponds to the vertical angle α of highest magnitude, is thus equal to 20 degrees, making this distribution appropriate for applications when, for example, an off-center but symmetrical distribution is desired. This type of distribution generally allows for greater spacing between adjacent lighting devices while maintaining a relatively uniform projection of light on the ground.

The direct-indirect distribution plot 1180 illustrated in FIG. 20E also takes the form of a two-dimensional polar graph that depicts the magnitude M of the intensity of the light output from a lighting device as a function of the vertical a from the horizontal. As shown in FIG. 20E, the distribution plot 1180 includes a first light distribution 1184 along a vertical plane through horizontal angles between 90-270 degrees, and a second light distribution 1188 measured along a vertical plane through horizontal angles between 180-0 degrees. As illustrated by the first and second light distributions 1184 and 1168, the magnitude M of light intensity is at its maximum value (in this example, 1398 candela) when the horizontal angle is 90 degrees and the vertical angle α is equal to 117.5 degrees, and most (e.g., approximately 80%) of the light is directed upwards (as evidenced by the fact that the light intensity is greater at vertical angles α between 90 degrees and 270 degrees. The main beam angle, which corresponds to the vertical angle α of highest magnitude, is thus equal to 117.5 degrees, making this distribution appropriate for applications when, for example, the lighting device is suspended from a ceiling and utilizes the ceiling to provide light to the environment, which in turn provides a low-glare lighting to the environment.

FIGS. 20E-20I each depict a chart that details the luminous flux (measured in lumens) for the lambertian, asymmetric, downlight with cutoff, and direct-indirect distributions 1120, 1140, 1160, and 1180, respectively. More specifically, each chart details the integration of the luminous intensity over the solid angle of the respective distribution 1120, 1140, 1160, and 1180, for various zones of vertical angles a (i.e., the luminous flux).

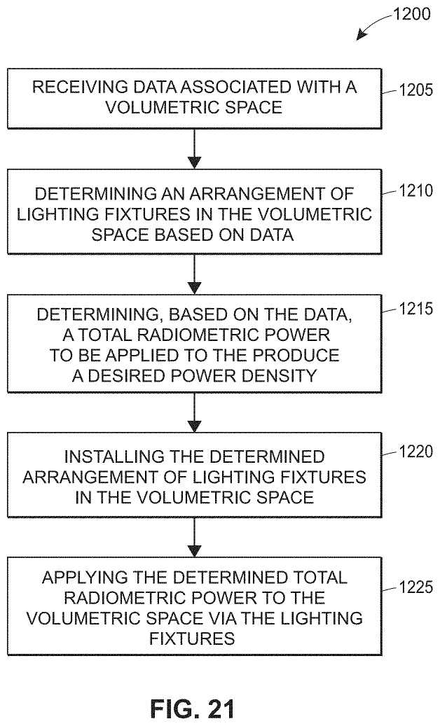

FIG. 21 depicts a flowchart of one method 1200 of providing doses of light sufficient to inactivate dangerous pathogens (e.g., SARS-CoV-2 virus, influenza A virus, MRSA bacteria, etc.) throughout a volumetric space (e.g., the environment 100) over a period of time (e.g., 24 hours). The method 1200 is implemented in the order shown, but may be implemented in or according to any number of different orders. The method 1200 may include additional, fewer, or different acts. For example, the first, second, third, and/or fourth data received in act 1205 may be received at different times prior to act 1220, with the receipt of data at different times constituting different acts. As another example, the acts 1205, 1210, and 1215 may be repeated a number of times before the act 1220 is performed.

The method 1200 begins when data associated with the volumetric space is received (act 1205). The data may include (i) first data associated with a desired illuminance level for the volumetric space, (ii) second data indicative of an estimated occupancy of the volumetric space over a pre-determined period of time, (iii) third data indicative of a length, width, and/or height of the volumetric space (one or more of the length, width, and/or height may be a default value, so need not be provided), and (iv) fourth data indicative of a preferred CCT for the volumetric space. While in this version the first, second, third, and fourth data is described as being received at the same time, these data can be received at different times. The desired illuminance level will vary depending upon the application and the size of the volumetric space, but may, for example, be 40-60 fc, 100-125 fc, 200-300 fc, or some other value or range of values. The estimated occupancy of the volumetric space over the pre-determined period of time generally relates to the amount of time per day that the volumetric space is occupied. Like the desired illuminance level, this will vary depending upon the application, but may be 4 hours, 6 hours, 8 hours, 12 hours, or some other period of time. The preferred CCT for the volumetric space will also vary depending upon the given application, but may, for example, be in a range of between approximately 1500 K and 7000 K, more particularly between approximately 1800 K and 5000 K.

The method 1200 includes determining an arrangement of one or more lighting fixtures to be installed in the volumetric space (act 1210). The determination is, in the illustrated method, based on the first data, though it can be made based on combinations of the first data, the second data, the third data, and/or the fourth data. The arrangement of one or more lighting fixtures generally includes one or more of any of the light fixtures described herein, e.g., the light fixture 200, light fixture 500, the light fixture 600, the light fixture 800, the light fixture 1000, and/or one or more other light fixtures (e.g., one or more light fixtures configured to emit only disinfecting light). Thus, the arrangement of one or more lighting fixtures is configured to at least partially provide or output (e.g., emit) disinfecting light (e.g., light having a wavelength of between 400 nm and 420 nm (e.g., about 405 nm), light having a wavelength of between 460 nm and 480 nm). In some cases, the one or more lighting fixtures may also be configured to at least partially provide light having a wavelength of greater than 420 nm (or greater than 500 nm), such that the combined or blended light output of the lighting fixtures is a more aesthetically pleasing or unobjectionable than would otherwise be the case. The arrangement of one or more lighting fixtures may also include means for directing the disinfecting light, such as, for example, one or more reflectors, one or more diffusers, and one or more lenses positioned within or outside of the lighting fixtures. The arrangement of one or more lighting fixtures may optionally include a means for managing heat generated by the one or more lighting fixtures, such that heat-sensitive components in the one or more lighting fixtures can be protected. The means for managing heat may, for example, take the form of one or more heat sinks and/or may involve utilizing a switching circuit that, when a lighting fixture that utilizes two light-emitting devices is employed, prevents the two circuits for the light-emitting devices from being energized at the same time during use. In some cases, a thermal cutoff may be added to prevent the lighting fixture(s) from overheating.

The method 1200 also includes determining a total radiometric power to be applied to the volumetric space via the one or more lighting fixtures so as to produce a desired power density at any exposed surface (i.e., unshielded surface) within the volumetric space during the period of time (act 1215). The determination is, in the illustrated method, based on the second data and third data, though it can be made based on combinations of the first data, the second data, the third data, and/or the fourth data. As discussed above, the desired power density may be or include a minimum irradiance equal to a value between 0.01 mW/cm² and 10 mW/cm², or preferably, between 0.01 mW/cm² and 1.0 mW/cm², as irradiance values above 1.0 mW/cm² are likely to exceed the exposure limit prescribed by the IEC 62471 standard. More particularly, the minimum irradiance may be equal to a value between 0.035 mW/cm² and 0.6 mW/cm², in view of the considerable virucidal effects of these irradiances as demonstrated in the studies described herein. The minimum irradiance may, for example, be equal to 0.01 mW/cm², 0.02 mW/cm², 0.035 mW/cm², 0.05 mW/cm², 0.076 mW/cm², 0.1 mW/cm², 0.15 mW/cm², 0.20 mW/cm², 0.25 mW/cm², 0.30 mW/cm², 0.35 mW/cm², 0.40 mW/cm², 0.45 mW/cm², 0.50 mW/cm², 0.55 mW/cm², 0.60 mW/cm², 0.65 mW/cm², 0.70 mW/cm², 0.75 mW/cm², 0.80 mW/cm², 0.85 mW/cm², 0.90 mW/cm², 0.95 mW/cm², 1.00 mW/cm², or some other value in the above-specified ranges. The minimum irradiance may be measured from any unshielded point in the volumetric space, a distance of 1.5 m from any external-most luminous surface of the lighting device, nadir, or some other point or surface in the volumetric space. In this manner, dangerous pathogens in the volumetric space are effectively inactivated.

In one example, the total radiometric power to be applied to the volumetric space can be determined according to the following formula: Total radiometric power =(Minimum irradiance (mW/cm²)*Duration (fractional day))/Volume of volumetric space (ft³), where the duration represents the amount of time per day that the volumetric space is to be occupied, and where the volume of the volumetric space is calculated by multiplying the length, height, and width of the volumetric space.

In some cases, e.g., when the arrangement of one or more lighting fixtures includes one or more lighting fixtures, such as the lighting fixtures 500, that are operable in different modes, the total radiometric power may be calculated for each of the modes and then summed to produce the total radiometric power to be applied to the volumetric space.

Once the total radiometric power to be applied to the volumetric space has been determined, the determined total may be compared to other applications (i.e., other volumetric spaces) for which disinfection levels have actually been measured, so as to verify that the total determined radiometric power for the volumetric space will be sufficient to inactivate dangerous pathogens.

The method 1200 then includes installing the determined arrangement of lighting fixtures in the volumetric space (act 1220), which can be done in any known manner, such that the determined total radiometric power can be applied to the volumetric space via the one or more lighting fixtures. The method 1200 optionally includes the act of applying the determined total radiometric power to the volumetric space via the one or more lighting fixtures (act 1225). By applying the determined total radiometric power, which is done without using any photosensitizers or reactive agents, produces the desired power density within the volumetric space during the period of time. In turn, dangerous pathogens (e.g., SARS-CoV-2, influenza A virus, MRSA bacteria, and/or other pathogens in accordance with the emitted disinfecting light) within the volumetric space are, over the designated period of time, inactivated by the specially arranged and configured lighting fixtures.

In some cases, act 1225 may also involve controlling the one or more light fixtures, which may done via one or more controllers (e.g., the controller 120, the controller 520) communicatively connected to the light fixtures. More specifically, the wavelength, the intensity, the bandwidth, or some other parameter of the disinfecting light (e.g., the light having a wavelength of between 400 nm and 420 nm) may be controlled or adjusted. This may be done automatically, e.g., when the one or more controllers detect, via one or more sensors, that the wavelength, the intensity, the bandwidth, or some other parameter of the disinfecting light has strayed, responsive to a control signal received from a central controller located remotely from the one or more lighting fixtures, and/or responsive to an input received from a user or operator of the lighting fixtures (e.g., entered via one of the client devices 70). In one example, the one or more light fixtures can be controlled responsive to new or altered first, second, third, and/or fourth data being received and/or detected (e.g., via a photo controller). In any event, such control or adjustment helps to maintain the desired power intensity, such that the one or more lighting fixtures continue to effectively inactivate dangerous pathogens throughout the volumetric space.

It will be appreciated that the volumetric space may vary in size depending upon the given application. As an example, the volumetric space may have a volume up to and including 25,000 ft³ (707.92 m³). In some cases, the volumetric space may be partially defined or bounded by a plane of the one or more lighting fixtures and a floor plane of the volumetric space. As an example, the volumetric space may be partially defined by an area that extends between 0.5 m below a plane of the one or more lighting fixtures and 24 in. (60.96 cm) above a floor plane of the volumetric space or an area that extends between 1.5 m below a plane of the one or more lighting fixtures and 24 in. (60.96 cm) above a floor plane of the volumetric space. The volumetric space may alternatively be defined by areas that are a different distance from the plane of the one or more lighting fixtures and/or the floor plane of the volumetric space.

Finally, it will be appreciated that the acts 1205, 1210, 1215, 1220, and 1225 of the method 1200 may be implemented by the server 66, one of the client devices 70, some other machine or device, a person, such as a user, a technician, an administrator, or operator, associated with the volumetric space, or combinations thereof.

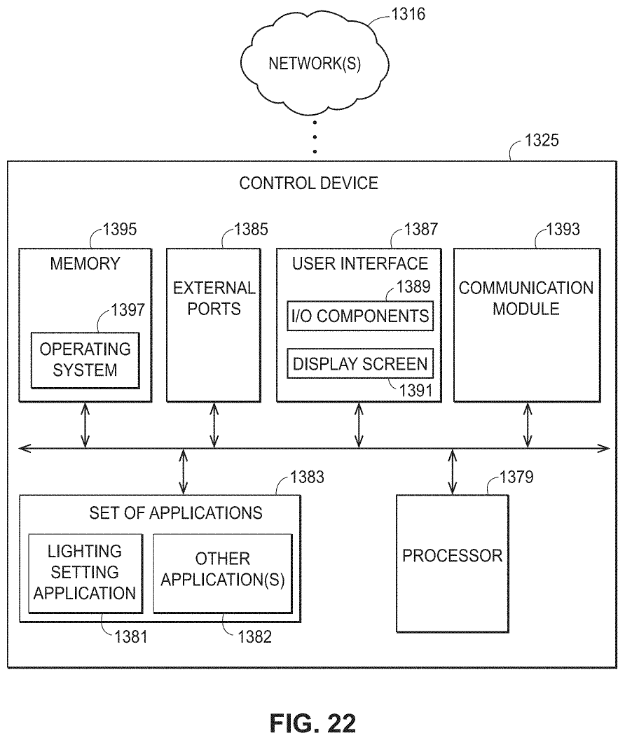

FIG. 22 illustrates an example control device 1325 via which some of the functionalities discussed herein may be implemented. In some versions, the control device 1325 may be the server 66 discussed with respect to FIG. 6, the local controller 120 discussed with respect to FIG. 7, the dosing feedback system 124 discussed with respect to FIG. 7, the local controller 520 discussed with respect to FIG. 14D, or any other control components (e.g., controllers) described herein. Generally, the control device 1325 is a dedicated machine, device, controller, or the like, including any combination of hardware and software components.

The control device 1325 may include a processor 1379 or other similar type of controller module or microcontroller, as well as a memory 1395. The memory 1395 may store an operating system 1397 capable of facilitating the functionalities as discussed herein. The processor 1379 may interface with the memory 1395 to execute the operating system 1397 and a set of applications 1383. The set of applications 1383 (which the memory 1395 may also store) may include a lighting setting application 1381 that is configured to generate commands or instructions to implement various lighting settings and transmit the commands/instructions to a set of lighting devices. It should be appreciated that the set of applications 1383 may include one or more other applications 1382.

Generally, the memory 1395 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The control device 1325 may further include a communication module 1393 configured to interface with one or more external ports 1385 to communicate data via one or more networks 1316 (e.g., which may take the form of one or more of the networks 74). For example, the communication module 1393 may leverage the external ports 1385 to establish a WLAN for connecting the control device 1325 to a set of lighting devices and/or to a set of bridge devices. According to some embodiments, the communication module 1393 may include one or more transceivers functioning in accordance with IEEE standards, 3GPP standards, or other standards, and configured to receive and transmit data via the one or more external ports 1385. More particularly, the communication module 1393 may include one or more wireless or wired WAN, PAN, and/or LAN transceivers configured to connect the control device 1325 to the WANs, PANs, and/or LANs.

The control device 1325 may further include a user interface 1387 configured to present information to a user and/or receive inputs from the user. As illustrated in FIG. 22, the user interface 1387 includes a display screen 1391 and I/O components 1389 (e.g., capacitive or resistive touch sensitive input panels, keys, buttons, lights, LEDs, cursor control devices, haptic devices, and others).

In general, a computer program product in accordance with an embodiment includes a computer usable storage medium (e.g., standard random access memory (RAM), an optical disc, a universal serial bus (USB) drive, or the like) having computer-readable program code embodied therein, wherein the computer-readable program code is adapted to be executed by the processor 1379 (e.g., working in connection with the operating system 1397) to facilitate the functions as described herein. In this regard, the program code may be implemented in any desired language, and may be implemented as machine code, assembly code, byte code, interpretable source code or the like (e.g., via C, C++, Java, Actionscript, Objective-C, Javascript, CSS, XML, and/or others).

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still cooperate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as examples and does not describe every possible embodiment, as describing every possible embodiment would be impractical, if not impossible. One could implement numerous alternate embodiments, using either current technology or technology developed after the filing date of this application. By way of example, and not limitation, the disclosure herein contemplates at least the following aspects:

1. A method of inactivating one or more lipid-enveloped viruses in an environment without an exogenous photosensitizer, the method comprising: providing light from at least one lighting element of a lighting device installed in the environment, the at least one lighting element configured to provide light toward a target area in the environment, the provided light having at least a virus-inactivating first component in a first range of wavelengths of 400 nanometers to 420 nanometers, wherein the virus-inactivating first component of light produces an irradiance of at least 0.01 mW/cm² and not more than 1.0 mW/cm² as measured at a surface in the target area that is unshielded from the lighting device and located at a distance of 1.5 meters from an external-most luminous surface of the lighting device, wherein providing the light causes the one or more lipid-enveloped viruses to be inactivated, and wherein the one or more lipid-enveloped viruses are inactivated without using the exogenous photosensitizer to cause inactivation of the one or more lipid-enveloped viruses.

2. The method of aspect 1, wherein the irradiance is at least 0.035 mW/cm² and not more than 0.6 mW/cm² at the surface in the target area that is unshielded from the lighting device and located at a distance of 1.5 meters from an external-most luminous surface of the lighting device.

3. The method of aspect 1 or 2, wherein the at least one lighting element comprises at least one light-emitting diode (LED).

4. The method of aspect 3, wherein the light is provided from the lighting device that further comprises a means for maintaining a junction temperature of the at least one LED below a maximum operating temperature of the at least one LED.

5. The method of any one of aspects 1 to 4, wherein the light is provided from the at least one lighting element that comprises: one or more first light-emitting elements configured to emit the virus-inactivating first component of the light; and one or more second light-emitting elements configured to emit a second component of the provided light, such that providing light from the at least one lighting element comprises providing a combined light formed by the first component of light in combination with the second component of light.

6. The method of aspect 5, wherein the combined light is white light having u′, v′ coordinates on the 1976 CIE Chromaticity Diagram that lie within an area that is bounded (i) vertically between 0.035 Duv below and 0.035 Duv above a planckian locus defined by the ANSI C78.377-2015 color standard, and (ii) horizontally between a correlated color temperature (CCT) isoline of between approximately 1500 K and 7000 K.

7. The method of aspect 6, wherein the area is bounded vertically between 0.007 Duv below and 0.007 Duv above the planckian locus.

8. The method of any one of aspects 1 to 7, wherein the at least one lighting element comprises: one or more light-emitting elements configured to emit the virus-inactivating first component of the light; and one or more light-converting elements arranged with respect to the one or more light-emitting elements such that (1) a first portion of the virus-inactivating first component of the light is not altered by the one or more light-converting elements, and (2) a second portion of the virus-inactivating first component of the light passes through the one or more light-converting elements to produce a second component of the provided light, the second component having a wavelength of greater than 420 nm, such that providing light from the at least one lighting element comprises providing a combined light formed by the first component of light in combination with the second component of light.

9. The method of aspect 8, wherein the combined light is white light having u′, v′ coordinates on the 1976 CIE Chromaticity Diagram that lie within an area that is bounded (i) vertically between 0.035 Duv below and 0.035 Duv above a planckian locus defined by the ANSI C78.377-2015 color standard, and (ii) horizontally between a correlated color temperature (CCT) isoline of between approximately 1500 K and 7000 K.

10. The method of aspect 9, wherein the area is bounded vertically between 0.007 Duv below and 0.007 Duv above the planckian locus.

11. The method of any one of aspects 8 to 10, wherein the one or more light-converting elements include one or more phosphors.

12. The method of any one of aspects 1 to 11, wherein the at least one lighting element is contained within a housing.

13. The method of aspect 12, wherein the lighting device further comprises means for creating air convection proximate to the housing.

14. The method of any one of aspects 1 to 13, wherein the lighting device further comprises means for directing the light provided by the at least one lighting element.

15. The method of any one of aspects 1 to 14, wherein a radiometric power of the provided light at 20 degrees from a center axis of light distribution is equal to 50% of a radiometric power at the center axis of light distribution of the provided light, wherein the radiometric power at 20 degrees and the radiometric power at the center axis are measured at equal distances from the at least one lighting element.

16. The method of any one of aspects 1 to 15, wherein the light provided by the at least one light-emitting element has a luminous flux above a cone angled downward from the lighting device at 60 degrees circumferentially around nadir of the lighting device, the luminous flux being greater than 15% of a total luminous flux of the light provided by the at least one lighting element.

17. The method of any one of aspects 1 to 16, wherein the light is provided from the at least one lighting element based upon instructions from a controller configured to control the at least one lighting element responsive to a control signal received from a user of the lighting device or from a central controller located remotely from the lighting device.

18. The method of any one of aspects 1 to 17, wherein the light is provided over an operating mode of 24 hours over which the lighting device is configured to irradiate the target area.

19. The method of any one of aspects 1 to 17, wherein the light is provided over an operating mode of eight hours over which the lighting device is configured to irradiate the target area.

20. The method of any one of aspects 1 to 19, in combination with any other suitable one of aspects 1 to 19.

21. A lighting system configured to inactivate one or more lipid-enveloped viruses in an environment without an exogenous photosensitizer, the lighting system comprising: a lighting device installed in the environment, the lighting device comprising at least one lighting element configured to provide light configured to provide light toward a target area in the environment, the provided light having at least a virus-inactivating first component in a first range of wavelengths of 400 nanometers to 420 nanometers, wherein the virus-inactivating first component of light produces an irradiance of at least 0.01 mW/cm² and not more than 1.0 mW/cm² as measured at a surface in the target area that is unshielded from the lighting device and located at a distance of 1.5 meters from an external-most luminous surface of the lighting device, and wherein the lighting system does not include an exogenous photosensitizer for causing inactivation of the one or more lipid-enveloped viruses, such that the providing of the light causes the one or more lipid-enveloped viruses to be inactivated without using the exogenous photosensitizer.

22. The lighting system of aspect 21, wherein the irradiance is at least 0.035 mW/cm² and not more than 0.6 mW/cm² at the surface in the target area that is unshielded from the lighting device and located at a distance of 1.5 meters from an external-most luminous surface of the lighting device.

23. The lighting system of aspect 21 or 22, configured to perform the method of any suitable one of aspects 1 to 20.

24. A method of inactivating one or more lipid-enveloped viruses in an environment without an exogenous photosensitizer, the method comprising: providing light from at least one lighting element of a lighting device installed in the environment, the at least one lighting element configured to provide light toward a target area in the environment, the provided light having at least a virus-inactivating first component in a first range of wavelengths of 400 nanometers to 420 nanometers, wherein the virus-inactivating first component of light produces an irradiance of at least 0.035 mW/cm² as measured at a surface in the target area that is unshielded from the lighting device and located at a distance of 1.5 meters from an external-most luminous surface of the lighting device, wherein providing the light causes the one or more lipid-enveloped viruses to be inactivated, and wherein the one or more lipid-enveloped viruses are inactivated without using an exogenous photosensitizer to cause the inactivation of the one or more lipid-enveloped viruses.

25. The method of aspect 24, in combination with the method of any one of aspects 1 to 20.

26. The method of aspect 24, implemented via the lighting system of any one of aspects 21 to 23.

27. Any one of aspects 1 to 26 in combination with any other suitable one of aspects 1 to 26.

Thus, many modifications and variations may be made in the techniques and structures described and illustrated herein without departing from the spirit and scope of the present claims. Accordingly, it should be understood that the methods and apparatus described herein are illustrative only and are not limiting upon the scope of the claims.

What is claimed:

1. A method of inactivating one or more lipid-enveloped viruses in an environment without an exogenous photosensitizer, the method comprising:
providing light from at least one lighting element of a lighting device installed in the environment, the at least one lighting element configured to provide light toward a target area in the environment, the provided light having at least a virus-inactivating first component in a first range of wavelengths of 400 nanometers to 420 nanometers,
wherein the virus-inactivating first component of light produces an irradiance of at least 0.01 mW/cm² and not more than 1.0 mW/cm² as measured at a surface in the target area that is unshielded from the lighting device and located at a distance of 1.5 meters from an external-most luminous surface of the lighting device,
wherein providing the light causes the one or more lipid-enveloped viruses to be inactivated, and
wherein the one or more lipid-enveloped viruses are inactivated without using an exogenous photosensitizer to cause inactivation of the one or more lipid-enveloped viruses.

2. The method of claim 1, wherein the irradiance is at least 0.035 mW/cm² and not more than 0.6 mW/cm² at the surface in the target area that is unshielded from the lighting device and located at a distance of 1.5 meters from an external-most luminous surface of the lighting device.

3. The method of claim 1, wherein the at least one lighting element comprises at least one light-emitting diode (LED).

4. The method of claim 3, wherein the light is provided from the lighting device that further comprises a means for maintaining a junction temperature of the at least one LED below a maximum operating temperature of the at least one LED.

5. The method of claim 1, wherein the light is provided from the at least one lighting element that comprises:
one or more first light-emitting elements configured to emit the virus-inactivating first component of the light; and
one or more second light-emitting elements configured to emit a second component of the provided light, such that providing light from the at least one lighting element comprises providing a combined light formed by the first component of light in combination with the second component of light.

6. The method of claim 5, wherein the combined light is white light having u′, v′ coordinates on the 1976 CIE Chromaticity Diagram that lie within an area that is bounded (i) vertically between 0.035 Duv below and 0.035 Duv above a planckian locus defined by the ANSI C78.377-2015 color standard, and (ii) horizontally between a correlated color temperature (CCT) isoline of between approximately 1500 K and 7000 K.

7. The method of claim 6, wherein the area is bounded vertically between 0.007 Duv below and 0.007 Duv above the planckian locus.

8. The method of claim 1, wherein the at least one lighting element comprises:
one or more light-emitting elements configured to emit the virus-inactivating first component of the light; and
one or more light-converting elements arranged with respect to the one or more light-emitting elements such that (1) a first portion of the virus-inactivating first component of the light is not altered by the one or more light-converting elements, and (2) a second portion of the virus-inactivating first component of the light passes through the one or more light-converting elements to produce a second component of the provided light, the second component having a wavelength of greater than 420 nm,
such that providing light from the at least one lighting element comprises providing a combined light formed by the first component of light in combination with the second component of light.

9. The method of claim 8, wherein the combined light is white light having u′, v′ coordinates on the 1976 CIE Chromaticity Diagram that lie within an area that is bounded (i) vertically between 0.035 Duv below and 0.035 Duv above a planckian locus defined by the ANSI C78.377-2015 color standard, and (ii) horizontally between a correlated color temperature (CCT) isoline of between approximately 1500 K and 7000 K.

10. The method of claim 9, wherein the area is bounded vertically between 0.007 Duv below and 0.007 Duv above the planckian locus.

11. The method of claim 8, wherein the one or more light-converting elements include one or more phosphors.

12. The method of claim 1, wherein the at least one lighting element is contained within a housing.

13. The method of claim 12, wherein the lighting device further comprises means for creating air convection proximate to the housing.

14. The method of claim 1, wherein the lighting device further comprises means for directing the light provided by the at least one lighting element.

15. The method of claim 1, wherein a radiometric power of the provided light at 20 degrees from a center axis of light distribution is equal to 50% of a radiometric power at the center axis of light distribution of the provided light, wherein the radiometric power at 20 degrees and the radiometric power at the center axis are measured at equal distances from the at least one lighting element.

16. The method of claim 1, wherein the light provided by the at least one light-emitting element has a luminous flux above a cone angled downward from the lighting device at 60 degrees circumferentially around nadir of the lighting device, the luminous flux being greater than 15% of a total luminous flux of the light provided by the at least one lighting element.

17. The method of claim 1, wherein the light is provided from the at least one lighting element based upon instructions from a controller configured to control the at least one lighting element responsive to a control signal received from a user of the lighting device or from a central controller located remotely from the lighting device.

18. The method of claim 1, wherein the light is provided over an operating mode of 24 hours over which the lighting device is configured to irradiate the target area.

19. The method of claim 1, wherein the light is provided over an operating mode of eight hours over which the lighting device is configured to irradiate the target area.

20. A lighting system configured to inactivate one or more lipid-enveloped viruses in an environment without an exogenous photosensitizer, the lighting system comprising:
a lighting device installed in the environment, the lighting device comprising at least one lighting element configured to provide light toward a target area in the environment, the provided light having at least a virus-inactivating first component in a first range of wavelengths of 400 nanometers to 420 nanometers,
wherein the virus-inactivating first component of light produces an irradiance of at least 0.01 mW/cm² and not more than 1.0 mW/cm² as measured at a surface in the target area that is unshielded from the lighting device and located at a distance of 1.5 meters from an external-most luminous surface of the lighting device,
and wherein the lighting system does not include an exogenous photosensitizer for causing inactivation of the one or more lipid-enveloped viruses, such that the providing of the light causes the one or more lipid-enveloped viruses to be inactivated without using an exogenous photosensitizer.

21. The lighting system of claim 20, wherein the irradiance is at least 0.035 mW/cm² and not more than 0.6 mW/cm² at the surface in the target area that is unshielded from the lighting device and located at a distance of 1.5 meters from an external-most luminous surface of the lighting device.

22. A method of inactivating one or more lipid-enveloped viruses in an environment without an exogenous photosensitizer, the method comprising:
providing light from at least one lighting element of a lighting device installed in the environment, the at least one lighting element configured to provide light toward a target area in the environment, the provided light having at least a virus-inactivating first component in a first range of wavelengths of 400 nanometers to 420 nanometers,
wherein the virus-inactivating first component of light produces an irradiance of at least 0.035 mW/cm² as measured at a surface in the target area that is unshielded from the lighting device and located at a distance of 1.5 meters from an external-most luminous surface of the lighting device,
wherein providing the light causes the one or more lipid-enveloped viruses to be inactivated, and
wherein the one or more lipid-enveloped viruses are inactivated without using an exogenous photosensitizer to cause the inactivation of the one or more lipid-enveloped viruses.
